(19) United States
(12) Patent Application Publication
(10) Pub. No.: US 2014/0243391 A1
(43) Pub. Date: Aug. 28, 2014

(54) PHOSPHOLIPID-DETERGENT CONJUGATES AND USES THEREOF

(71) Applicants: Luc Lebeau, Fegersheim (FR); Philippe Pierrat, Vandoeuvre-Les-Nancy (FR); Francoise Pons, Fegersheim (FR); Guy Zuber, Illkirch (FR)

(72) Inventors: Luc Lebeau, Fegersheim (FR); Philippe Pierrat, Vandoeuvre-Les-Nancy (FR); Francoise Pons, Fegersheim (FR); Guy Zuber, Illkirch (FR)

(73) Assignees: CENTRE NATIONAL DE LA RECHERCHE SCIENTIFIQUE, PARIS (FR); UNIVERSITE DE STRASBOURG, STRASBOURG (FR)

(21) Appl. No.: 14/233,797

(22) PCT Filed: Jul. 20, 2012
(86) PCT No.: PCT/EP2012/064276

§ 371 Date: Apr. 18, 2014

Related U.S. Application Data
(60) Provisional application No. 61/510,531, filed on Jul. 22, 2011.

(51) Int. Cl. A61K 48/00; C12N 15/113; A61K 47/24

(52) U.S. Cl. CPC A61K 48/0025; A61K 47/24; C12N 15/113; C12N 2310/14; C12N 2320/30
USPC 514/44 A; 558/172; 435/375

(57) ABSTRACT

The invention relates to novel compounds, in particular novel O-substituted phospholipids that are useful for the in vitro and in vivo delivery of drugs as well as nucleic acids into cells. The invention also relates to pharmaceutical compositions and supramolecular complexes comprising said compounds and the use of these compounds in therapeutic treatment, in particular in gene therapy.

### FIELD OF THE INVENTION

The invention relates to novel compounds, in particular novel O-substituted phospholipids with detergent moiety that are useful for the in vitro and in vivo delivery of drugs as well as nucleic acids into cells. The invention also relates to pharmaceutical compositions and supramolecular complexes comprising said compounds and the use of these compounds in therapeutic treatment, in particular in gene therapy.

### BACKGROUND OF THE INVENTION

Gene therapy to correct genetic deficiencies or treat acquired diseases has emerged as a promising approach over the last two decades. However, the delivery of nucleic acids to cells relies on carriers to improve transfection efficiency. Cationic lipids and polymers were introduced more than twenty years ago to deliver nucleic acid to cells [1, 2]. Their use is based on the hypothesis that lipoplex and polyplex (i.e. particles resulting from the condensation of a nucleic acid by a cationic lipid or a cationic polymer, respectively), with regard to the excess of cationic charges involved, may interact with the negatively charged plasma membrane of mammalian cells via electrostatic interactions. Such an interaction may trigger different internalization processes and, once inside cells, the transfecting particles are expected to safely release their nucleic acid payload in the cytosol before its migration to the nucleus for genetic expression.

The discovery of RNA interference (RNAi) ten years ago has opened a new gateway to applications in gene therapy [3]. Synthetic double-stranded RNA sequences of 21-23 nucleotides have the potential to specifically downregulate gene function in mammalian cells [4]. In the cytosol, these small interfering RNAs (siRNAs) associate with a nucleic acid-protein complex called RNA-induced silencing complex (RISC) that mediates a sequence specific downregulation of a complementary messenger RNA in a temporally and spatially regulated manner. As the site of action of siRNA is in the cytosol, there is no requirement to enter the nucleus for controlling the expression of target genes. Though there is no immediate correlation between the efficiency of a synthetic vector used for DNA and siRNA delivery, the technologies developed for delivery of nucleic acids such as plasmid DNA have paved the way to rapid progress for in vivo delivery of siRNA.

A large number of studies have focused on the different events occurring from the nucleic acid condensation mediated by the vector to the transgene expression in order to improve transfection efficiency. However, most of these processes involving lipoplex or polyplex remain unsatisfactorily understood [5-11] and are inefficient. Currently, it appears that in vitro only ca. 10-15% of particles are internalized, cell uptake proceeding essentially through endocytosis pathways [12]. Less than 5% of the endocyted particles escape endosomal compartment before degradation [13].

In the case of DNA delivery, released complexes still have to diffuse in the cytosol towards the nucleus and cross the nuclear membrane. That is achieved only by a tiny amount of DNA particles, most likely owing to cell division events. As cell culture experiments employ rapidly dividing cells cultured in monolayers, they do not accurately mimic the in vivo situation (slow- or non-dividing cells) and these latter quantities are drastically decreased when transfection experiments are carried out in vivo. RNA delivery may therefore be more suitable for transient gene expression, particularly in non-dividing cells [14].

Different strategies have been developed to improve cellular uptake of transfecting particles. They essentially involve decoration of the particles with biocompatible polymers (polyethylene glycol, PEG) to extend circulation time, and with specific ligands for cell targeting [5, 9, 15]. The endocytosis route is generally considered as the predominant pathway for the uptake of particles into the cells [6, 13, 16, 17]. The entrapment of the internalized transfection particles in endocytic compartments prevents further intracellular transport and will often result in degradation of the carrier and its associated nucleic acid in the endosomal/lysosomal compartments [18]. Thus efficient delivery systems face the need to—at least partly—avoid the lysosomal degradation machinery and to create cytosolic delivery [11].

The early endosome acts as the first sorting station in the endosomal pathway. It is a dynamic compartment with high homotypic fusion capacity [19] and it has been suggested that endocytic sorting (toward recycling or degradation pathways) is efficiently based on membrane physical properties [20]. Membrane insertion of destabilizing (cone-shape) compounds favors high membrane curvature and vesicular fission that triggers recycling to the cell membrane, with cytosolic release of the vesicular content. At the opposite, membrane rigidifying lipids preferentially target traffic along the degradation pathway. Destabilization of the plasma and/or endosomal membrane(s) by the transfecting particles or fusion with the latter is thus a key step for efficient cytosolic delivery.

Destabilization of the plasma or endosomal membrane may be induced e.g. by fusogenic lipids [21] or membrane-active peptides displayed at the periphery of lipoplex [22]. Protonation of carriers bearing amine groups with a pKₐ within the physiologic range of 4.5 to 8 upon regular acidification of the endosomal content provokes osmotic swelling of the compartment (proton sponge effect) that may end in lysis as well [23]. Those nucleic acid carriers that manage to escape the endosomal compartment are then challenged by the complex environment of the cytosol, which contains many filamentous structures that impede the free diffusion of large particles. Dissociation from the carrier at this stage might be required to allow further transport of the nucleic acid molecule before it is fully degraded by cytosolic nucleases [24]. In the case of lipoplexes, it has been demonstrated that mixing of cationic lipids with anionic membrane lipids (e.g. phosphatidylserine, phosphatidylglycerol and phosphatidylinositol) induced destabilization of the complexes and nucleic acid release [25]. This is presumably a consequence of electrostatic interactions between the cationic and anionic lipids that compete with the binding reaction between the cationic lipids and the nucleic acid.

Membrane disruptive properties of detergents have been early considered for improving nucleic acid delivery. Cationic detergents have been shown to provoke DNA condensation [26, 27] but the resulting lipoplex revealed to be inactive in vitro, or only poorly active when particles were prepared from cationic detergent mixed with 1,2-dioleoyl-sn-glycero-3-phosphoethanolamine (DOPE) [28, 29]. That is explained by the rapid exchange of the detergent molecules between the complex with nucleic acid and the aqueous environment (due to the high critical micellar concentration—cmc—value) resulting in irreversible decondensation of nucleic acid before efficient entry into the cells [30]. However recent reports indicate that lipoplexes containing different cationic lipids and Tween 80™ as a modifier may exhibit enhanced transfection properties both in vitro and in vivo [31-33].

Thus, it is apparent that a need exists for identifying new molecules, particularly lipid molecules, which can act as effective carriers for nucleic acid delivery while demonstrating low cytotoxic activity and resistance to intracellular degradation.

### BRIEF SUMMARY OF THE INVENTION

The invention relates to a compound of formula (I):

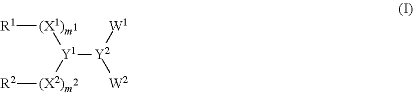

or a salt thereof, wherein:
R¹ and R² are independently selected from the groups consisting of linear, unsaturated or saturated, C₈ to C₃₀ alkyl groups eventually interrupted by one or several heteroatoms and eventually substituted by one or several groups selected from C₁-C₃ alkyl groups, halogens, —OH, —OMe, and —CF₃,
X¹ and X² are independently selected from the group consisting of —O—, —OC(O)—, —C(O)O—, —OC(O)O—, —S—, —SS—, —SC(O)—, —OC(S)—, —NR³—, —NR³C(O)—, —C(O)NR³—, —NR³C(S)—, —C(S)NR³—, —OC(O)S—, —OC(S)O—, —SC(O)O—, —OC(S)S—, —SC(O)S—, —SC(S)O—, —SC(S)S—, —OC(O)NR³—, —OC(S)NR³—, —NR³C(S)O—, —NR³C(O)S—, —NR³C(O)NR⁴—, —NR³C(S)NR⁴—, —SC(O)S—, —SC(S)O—, —S(O)—, —S(O)₂—, —O(CR³R⁴)O—, —C(O)O(CR³R⁴)O—, —OC(O)O(CR³R⁴)O—, —P(O)(R³)—, —P(O)(OR³)—, —P(O)(R³)O—, —OP(O)(OR³)—, —OP(O)(R³)O—, —NR³P(O)(R⁴)—, —NR³P(O)(OR⁴)—, —NR³P(O)(R⁴)O—, —OP(O)(OR³)— and —OP(O)(R³)O— wherein R³ and R⁴ are independently H or CH₃,
m¹ and m² are integers independently selected from 0 and 1,
Y¹ and Y² are trivalent connectors selected from the group consisting of N<, —CON<,

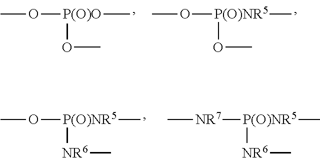

and linear or branched alkyl C₁-C₁₀ groups, wherein R⁵, R⁶ and R⁷ are independently selected from H and CH₃,
W² is a straight or branched radical comprising from 2 to 20 carbon atoms and at least one functional group selected from ester, carboxylate, —OH, ether, primary, secondary, tertiary or quaternary amine, and combinations thereof,
W¹ is a radical having the following formula (II):

-(L)ₛ-(ZO)ₙ—R⁸ wherein
s is 0 or 1,
n is an integer from 0 to 30, preferably from 0 to 22, with the proviso that n is not 0 when s is 0.
L is —C(R²⁰)(R²¹)—O—C(O)—, —C(R²²)(R²³)—O—C(O)—O— or —C(R²⁴)(R²⁵)—O—C(O)—(CH₂)ₚ—C(O)O— wherein R²⁰ to R²⁵ are selected from the group consisting of H and C₁-C₃ alkyl groups, which may be linear, cyclic or branched, and, p is an integer between 1 to 10,
Z is —CH₂CH₂—, —CH₂CH₂CH₂— or —CH₂CH₂CH₂CH₂— and
R⁸ is selected from the group consisting of:
   Unsaturated or saturated, linear, C₁-C₂₄ alkyl groups, preferably C₅-C₂₄ groups, optionally substituted with one or several groups selected from —F, —Cl, —Br, —I, —OH, —OMe, C₁-C₄ alkyl groups and —CF₃ and optionally interrupted by an heteroatom.
   An aryl group substituted by one or several C₁-C₁₂ linear or branched alkyl groups, preferably

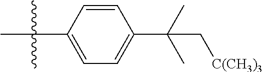 [SMILES: *c1ccc(C(C)(C)CC(C)(C)C)cc1]

Preferably the said compound has the following formula (IV)

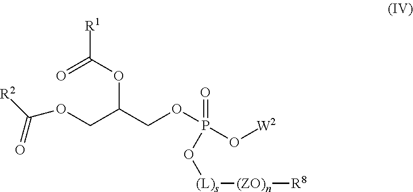

In some embodiments, the said compound is characterized in that:
s=1 and
L is selected from the group consisting of —CH(R²⁰)—O—C(O)—, —CH(R²²)—O—C(O)—O— or —CH(R²⁴)—O—C(O)—(CH₂)ₚ—C(O)O— wherein R²⁰, R²², and R²⁴ are selected from the group consisting of H and C₁-C₃ alkyl groups, preferably selected from the group consisting of —CH₂—O—C(O)—(CH₂)₂—C(O)O—, CH₂—O—C(O)—, and —CH(R²²)—O—C(O)—O— wherein R²² is —H, —CH₃ or —CH(CH₃)₂.

In some embodiments, the compound of formula (IV) or (I) is such that R⁸ is selected from the group consisting of:
—(CH₂)ₓCH₃ with x an integer from 0 to 23, preferably from 0 to 16, still more preferably from 4 to 16, and
—(CH₂)_{y}—CH═CH—(CH₂)_{z}—CH₃ with z and y are integers such that 2≦y+z≦21.

Said compound may be further characterized by one of the following combinations of features:

i) s=0, Z=—CH₂—CH₂— and n is an integer from 1 to 8, preferably 2 to 8; and
ii) s=1, Z=—CH₂—CH₂—, n is an integer from 0 to 8 and L is —CH₂—O—C(O)—, —CH₂—O—C(O)—O—, —CH(CH₃)—O—C(O)—O—, —CH(iPr)—O—C(O)—O— and —CH₂—O—C(O)—CH₂—CH₂—C(O)—O—.

In a particular embodiment, the compound of formula (IV) or (I) is such that R⁸ is

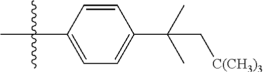 [SMILES: *c1ccc(C(C)(C)CC(C)(C)C)cc1]

Said compound may further have:
Z is —CH₂—CH₂—,
n is an integer from 5 to 10, preferably 6 to 10, and
s=0 or s=1 with the proviso that L is —CH₂—O—C(O)—(CH₂)₂—C(O)—O—, —CH(CH₃)—O—C(O)O— or CH₂—O—C(O)O—.

In some embodiments, W² is selected from the group consisting of:
—CH₂—CH(COOH)NH₂,
—CH₂—CH(OH)—CH₂—OH,
a straight or branched oligoethylenimine comprising from 2 to 6 monomers,
—(Z¹NR¹⁴)_{q}—R¹⁵ and
—Z¹NR¹⁶R¹⁷R¹⁸⁺ Q⁻
   wherein Z¹ is the same or different and is selected from the group consisting of —(CH₂)₂—, —(CH₂)₃— or —(CH₂)₄—, q is an integer from 1 to 4, R¹⁴ to R¹⁸ is H or CH₃ and a Q⁻ is a pharmaceutically acceptable anion.

In some other embodiments R¹ and R² are independently selected from the group consisting of unsubstituted and straight C¹²-C²⁴ alkyl groups comprising 0, 1, 2, 3 or 4 unsaturations. In some specific embodiments, R¹ and R² are CH₃—(CH₂)₇—CH═CH—(CH₂)₇— and W² is —CH₂—CH₂—N(CH₃)₃⁺Q-.

Another object of the invention is a supramolecular complex comprising one or several compounds according to the invention and a pharmaceutically active compound.

A further aspect of the invention is a pharmaceutical composition comprising a pharmaceutically active compound, a compound according to the invention and optionally a pharmaceutically acceptable excipient.

In the supramolecular complex or the pharmaceutical composition of the invention, the active compound may be a nucleic acid molecule, preferably a siRNA or a DNA.

The invention also relates to a compound as defined above, for use as a delivering agent for the administration of a pharmaceutically active compound to an animal, preferably a mammal. Another object of the invention is a method for delivering a molecule of interest to a cell, preferably a pharmaceutically active compound, said method comprising contacting a pharmaceutical composition or a supramolecular complex as defined above with said cell. In some embodiments, the said method is performed in vitro and/or ex vivo.

### BRIEF DESCRIPTION OF THE DRAWINGS

FIG. 1. Transmission electron microscopy (TEM) image of siRNA/conjugate 1 (PP168) complexes (scale bar: 100 nm).

FIG. 2. Permeation effect of investigated lipids on mammalian cells membrane. Sheep erythrocytes were incubated for 2 h at 37° C. with increasing amounts of lipids in PBS, pH 7.4 (conjugate 1 (PP168): filled square; EDOPC: empty triangle; EDOPC/TX100: filled triangle; TX100: circle). Cells were then centrifuged and hemolysis was assessed by monitoring hemoglobin release in the supernatant. Data shown are representative of a triplicate determination (mean±SD).

FIG. 3. Gene knockdown activity of cationic DOPC conjugates in U87-Luc cells. Silencing effect was expressed as the percentage of luciferase activity in the anti-luciferase siRNA (siLuc) treatment compared to that in the anti-GFP siRNA (sic) treatment used as a negative control (typical luciferase luminescence signal around 1.5 10⁷ RLU/mg protein). Lipoplexes were prepared from 1.0 pmol siRNA and 1.0 (white), 2.0 (grey), or 4.0 nmol (black) of cationic lipids, where 1 refers to conjugate 1 (PP168), 2 refers to conjugate 6 (PP338), and EDOPC. EDOPC/TX100 refers to an equimolar mixture of EDOPC and TX100. Experiments were carried out on 96-well plates (8.000 cells/well) at 10 nM siRNA final concentration (lipid concentration: 10, 20, and 40 μM). Luciferase activity was measured as indicated in Supplementary Information. Data are represented as the mean±SD of triplicates.

FIG. 4. Lipoplex cytotoxicity as determined by the LDH release assay. Cytotoxicity was evaluated on 16HBE cells after 48 h incubation in the presence of lipoplexes prepared from 1.0 pmol siRNA and increasing amounts of lipids (white: 1.0 nmol; grey: 2.0 nmol; black: 4.0 nmol). Compound 1 refers to PP168. EDOPC/TX100 refers to the equimolar mixture of the two compounds. Basal LDH is set at 0%, and 100% represents the total LDH released after cell lysis. Data shown are representative of a triplicate determination (mean±SD).

FIG. 5. Luciferase silencing in the U87-Luc cell line by siRNA complexed with conjugate 1 (PP168)/EDOPC at various ratio. Lipoplexes were prepared from 1 pmol siRNA and 1, 2, or 4 nmol of cationic lipids. Cells were transfected with non-specific (sic; white bars, negative control) or luciferase-specific (siLuc; black bars) siRNA. Luciferase expression in untreated cells was taken as 100%. Data shown are representative of a triplicate determination (mean±SD).

FIG. 6. DNA binding ability of the phospholipid-detergent conjugates 1-5 (1: PP168; 2: PP111; 3: PP163; 4: PP299; 5: PP303) at increasing charge ratio (N/P). One μg of plasmid DNA and increasing amounts of cationic lipid were each diluted in 25 μL of 150 mM NaCl and gently mixed. After an incubation period of 20 min, samples (25 μL) were analyzed by electrophoresis through a 1% agarose gel using Tris-borate-EDTA buffer and DNA was visualized after SYBR Safe (Invitrogen) staining.

FIG. 7. Hemolytic properties on mammalian cell membrane of lipid 1 (PP168) and EDOPC+TX100, and of the lipoplexes made thereof at a N/P ratio of 25. Sheep erythrocytes were incubated with increasing amount of lipids and hemolysis was monitored by release of hemoglobin in the supernatant. Data shown are representative of a triplicate determination (mean±SD).

FIG. 8. Metabolic degradation of conjugate 1 (PP168). siLuc complex with conjugate 1 was incubated with cells for 5, 10, 24, and 48 h before lipid extraction and Maldi-ToF MS analysis of extracts. m/z for the major oligomer of 1 is 1239.

FIG. 9. Metabolic degradation of conjugate 2 (PP111). siLuc complex with conjugate 2 was incubated with cells for 5, 10, 24, and 48 h before lipid extraction and Maldi-ToF MS analysis of extracts. m/z for the major oligomer of 2 is 1401.

FIG. 10. Metabolic degradation of conjugate 3 (PP163). siLuc complex with conjugate 3 was incubated with cells for 5, 10, 24, and 48 h before lipid extraction and Maldi-ToF MS analysis of extracts. m/z for the major oligomer of 3 is 1459.

FIG. 11. Metabolic degradation of conjugate 4 (PP299). siLuc complex with conjugate 4 was incubated with cells for 5, 10, 24, and 48 h before lipid extraction and Maldi-ToF MS analysis of extracts. m/z for the major oligomer of 4 is 1457.

FIG. 12. Metabolic degradation of conjugate 5 (PP303). siLuc complex with conjugate 5 was incubated with cells for 5, 10, 24, and 48 h before lipid extraction and Maldi-ToF MS analysis of extracts. m/z for the major oligomer of 5 is 1515.

FIG. 13. Metabolic degradation of EDOPC. siLuc complex with EDOPC was incubated with cells for 5, 10, 24, and 48 h before lipid extraction and Maldi-ToF MS analysis of extracts. Expected m/z for EDOPC is 814.6.

FIG. 14. Luciferase silencing by siRNA-Luc complexed with the cationic lipids 1-6 (1: PP168; 2: PP111; 3: PP163; 4: PP299; 5: PP303; 6: PP338), EDOPC, and DOTAP, in the U87-Luc cell line. EDOPC+TX100 refers to an equimolar mixture of EDOPC and TX100. Experiments were carried out on 96-well plates (8.000 cells/well) at 10 nM siRNA final concentration. Lipoplex were prepared from 1.0 pmol siRNA and 1 nmol (white), 2 nmol (grey), or 4 nmol (black) of cationic lipid. Luciferase activity was measured as indicated in Methods. Data are represented as the ratio between specific (siRNA-Luc) and non-specific (siRNA-eGFP) response to take into account the variations of metabolic activity of the cells. Data are means±SD of triplicates.

FIG. 15. Mitochondrial activity of 16HBE cells evaluated by the MTT assay after 48-h incubation in the presence of lipoplexes prepared from 1.0 pmol siRNA and increasing amounts of lipids (white: 1.0 nmol; grey: 2.0 nmol; black: 4.0 nmol) (1: PP168; 2: PP111; 3: PP163; 4: PP299; 5: PP303). EDOPC+TX100 refers to the equimolar mixture of the two compounds. Data shown are representative of triplicate determinations (mean±SD).

FIG. 16. Lipoplexes cytotoxicity on 16HBE cells as determined by the LDH release assay. Cytotoxicity was evaluated after 48-h incubation in the presence of lipoplexes prepared from 1.0 pmol siRNA and increasing amount of lipid (white: 1.0 nmol; grey: 2.0 nmol; black: 4.0 nmol) (1: PP168; 2: PP111; 3: PP163; 4: PP299; 5: PP303). EDOPC+TX100 refers to the equimolar mixture of the two compounds. Basal LDH production is set at 0%, and 100% represents the total LDH released after cell lysis. Data shown are representative of a triplicate determination (mean±SD).

FIG. 17. Permeation effect of investigated lipids on mammalian cell membrane. Sheep erythrocytes were incubated with increasing amount of lipids (square: 1, PP168; circle: 2, PP111; filled losange: 3, PP163; filled triangle: EDOPC; triangle: TX100; filled square: EDOPC+TX100) and hemolysis was monitored by release of hemoglobin in the supernatant. Data shown are representative of a triplicate determination (mean±SD)

FIG. 18: Expression of luciferase in BHK-21 cells treated with pCMVLuc pDNA complexed with the compounds of the invention described in Part B of the examples or EDOPC, in the presence of 10% FCS. Lipoplexes were prepared at various charge ratios (N/P: 1.0: black, 3.0: grey, 5.0: white). Control (C) refers to basal luminescence measured in untreated cells. Data shown are representative of a triplicate determination (mean±SD). Compound 1: PP94; 2: PP140; 3: PP138; 4: PP189; 5: PP194, 6: PP91, 7: PP120, 8: PP178, and 9: PP93. Y-coordinate: Luciferase expression in Log scale (RLU/well).

FIG. 19 shows the expression of luciferase (left—Y-coordinate: RLU/well in Log scale) and LDH release (right—Y-coordinate: % of LDH release) in A549 (FIG. 19A), Calu-3 (FIG. 19B), and NCI-H292 (FIG. 19C) cells treated with pCMVLuc pDNA complexed with the compounds of the invention 1-9 or EDOPC, in the presence of 10% FCS. Lipoplexes were prepared at various charge ratios (N/P: 1.0: black, 3.0: grey, 5.0: white). Control (C) refers to basal luminescence measured in untreated cells. Basal LDH is set at 0%, and 100% represents the total LDH released after cell lysis. Data shown are representative of a triplicate determination (mean±SD). Compound 1: PP94; 2: PP140; 3: PP138; 4: PP189; 5: PP194, 6: PP91, 7: PP120, 8: PP178, and 9: PP93.

FIG. 20 shows the efficiency of cationic lipids 1-9 (1: PP94; 2: PP140; 3: PP138; 4: PP189; 5: PP194, 6: PP91, 7: PP120, 8: PP178, and 9: PP93), as compared to reference EDOPC, to assist siRNA delivery in U87 cells that were stably transformed to express the egfpluc fusion protein. The culture medium contained 10% FBS. Each lipid was mixed with either untargeting (control) siRNA (sic, black) or luc-targeting siRNA (siLuc, grey) and added to cells. Luciferase activity was measured 48 h later and plotted relative to untreated cells (100% luciferase expression, data not shown). Lipoplex were prepared from 1.0 pmol siRNA and 1 nmol, 2 nmol, or 4 nmol of cationic lipid. Data shown are representative of a triplicate determination (mean±SD).

### DETAILED DISCLOSURE OF THE INVENTION

### I. Compounds According to the Invention

The present invention relates to lipid conjugates useful for delivering active substances.

The lipid conjugates according to the invention comprises three essential features:
A hydrophobic tail comprises two lipophilic chains called R¹ and R²;
A head comprising a hydrophilic moiety W² and
A detergent moiety, called hereunder W¹, which comprises a spacer conjugated to a hydrophobic group wherein the said spacer may comprise a polyether, an oligoether or a etheroxide moiety and/or a hydrolyzable connector. The hydrophobic group is preferably

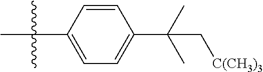 [SMILES: *c1ccc(C(C)(C)CC(C)(C)C)cc1]

or a C₁-C₂₄ alkyl group, preferably a C₅-C₂₄ alkyl group, and derivatives thereof.

The compounds of the invention may be used for promoting in vitro or in vivo delivery of active compounds into cells, in particular nucleic acid molecules such as plasmid DNA or siRNA.

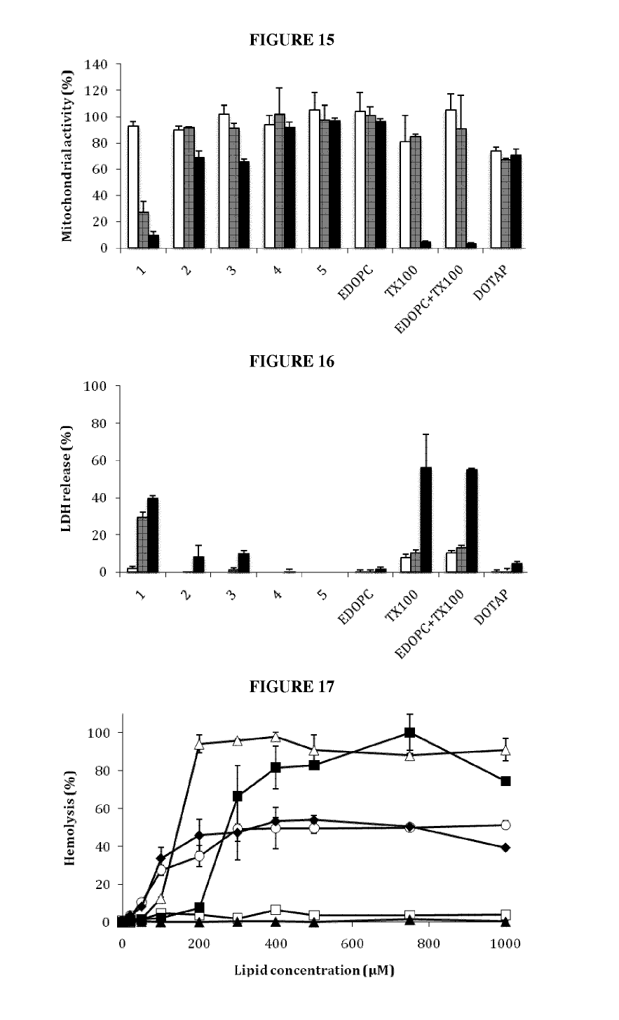

In a first series of experiments, the Applicant showed that the covalent coupling of Triton X-100® to the phosphate group of 1,2-dioleoyl-sn-glycero-3-phosphocholine (DOPC) provided a cationic phospholipid conjugate able to complex a nucleic acid molecule and transfect it into cell. Surprisingly, the resulting conjugate is significantly more efficient than EDOPC and a mixture of EDOPC and Triton X-100® (1:1) to transfect anti-luciferase siRNA in U87-LUC cell and induce luciferase activity knockdown. Notably, the transfection activity of the conjugate results, at least partially, from the 2-(2,4,4-trimethyl)pentyl phenyl moiety. Indeed, the conjugate resulting from the covalent coupling of DOPC with PEG block failed to knock down luciferase gene (see FIG. 3). The applicant further showed that the introduction of a hydrolyzable connector linking phosphate group to 2-(2,4,4-trimethyl)pentyl phenyl moiety may significantly decrease the cell toxicity of the compound, as shown by LDH release assay and MTT assay (see FIGS. 15 and 16).

In a second series of experiments, the Applicant showed that the 2-(2,4,4-trimethyl)pentyl moiety may be replaced by other hydrophobic groups, namely alkyl chains such as —(CH₂)₁₁CH₃. The resulting compounds—which may comprise a hydrolyzable connector and/or an oligoethylene glycol spacer linking the phosphate group to the said alkyl chain—are able to promote the transfection of plasmid DNA in various cell lines. Moreover, some compounds of the invention are able to transfer both siRNA and DNA into cells.

Surprisingly, the compounds of the invention—which comprise a hydrolyzable connector and/or a PEG spacer—may display higher transfection activity than phospholipid conjugates in which the alkyl chain is directly linked to the phosphate group. Moreover, the Applicant further showed that the said compounds generally exhibit very low LDH release activity. Accordingly, these compounds are expected to display low toxicity in vivo and thus to be better tolerated than other synthetic vectors described in the prior art.

A first aspect of the invention pertains to a compound of formula (I)

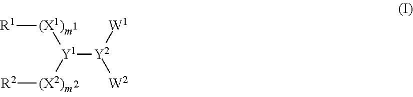

wherein:
R¹ to R¹⁸ may be identical or different and may be selected from the group consisting of: hydrogen, a linear or branched C₁ to C₃₀ alkyl that may contain one or several double bond, triple bond, cycle, or heteroatom, preferably R¹═R²=cis-nC₈H₁₇CH═CHC₇H₁₄—;
X¹ and X² are divalent connectors, may be identical or different and may be selected from the group consisting of: —O—, —OC(O)—, —C(O)O—, —OC(O)O—, —S—, —SS—, —SC(O)—, —OC(S)—, —C(S)O— —NR³—, —NR³C(O)—, —C(O)NR³—, —NR³C(S)—, —C(S)NR³—, —OC(O)S—, —OC(S)O—, —SC(O)O—, —OC(S)S—, —SC(O)S—, —SC(S)O—, —SC(S)S—, —OC(O)NR³—, —OC(S)NR³—, —NR³C(S)O—, —NR³C(O)S—, —NR³C(O)NR⁴—, —NR³C(S)NR⁴—, —SC(O)S—, —SC(S)O—, —S(O)—, —S(O)₂—, —O(CR³R⁴)O—, —C(O)O(CR³R⁴)O—, —OC(O)O(CR³R⁴)O—, —P(O)(R³)—, —P(O)(OR³)—, —P(O)(R³)O—, —OP(O)(OR³)—, —OP(O)(R³)O—, —NR³P(O)(R⁴)—, —NR³P(O)(OR⁴)—, —NR³P(O)(R⁴)O—, —OP(O)(OR³)—, —OP(O)(R³)O—, preferably X¹=X²=—C(O)O—;
m¹ and m² are integers, may be identical or different, and may be selected from the group consisting of: 0 or 1, preferably m¹=m²=1;

Y¹ and Y² are trivalent connectors, may be identical or different and may be selected from the group consisting of: a linear alkyl group and a branched alkyl group (that may contain one or several double bond, triple bond, cycle or heteroatom), —N<, —CON<,

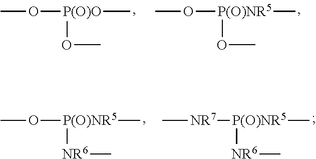

preferably:

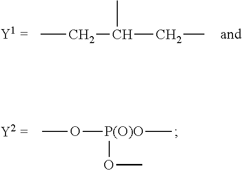

W¹ and W² may be identical or different and may be selected from the group consisting of:
   —(ZO)ₙ—R⁸, —(CR⁹R¹⁰)O(ZO)ₙ—R⁸,
   —(CR⁹R¹⁰)OC(O)O(ZO)ₙ—R⁸,
   —(CR⁹R¹⁰)OC(O)(CR¹¹R¹²)ₚC(O)O(ZO)ₙ—R⁸,
   —(CR⁹R¹⁰)OC(O)(CR¹¹R¹²)ₚO(ZO)ₙ—R⁸,
   —(CR⁹R¹⁰)OC(O)(CR¹¹R¹²)ₚC(O)NR¹³(ZO)n—R⁸,
   —(ZNR¹⁴)_{q}—R¹⁵,
   —ZNR¹⁶R¹⁷R¹⁸⁺Q;
   preferably W² is —C₂H₄NMe₃⁺Q⁻;
Z may be selected from the group consisting of: a linear or branched C₁ to C₃₀ alkylyl group that may contain one or several double bond, triple bond, cycle or heteroatom), preferably Z is a linear C₂ to C₄ alkylyl groups: —CH₂CH₂—, —CH₂CH₂CH₂—, —CH₂CH₂CH₂CH₂—; Q⁻ may be selected from the group consisting of: OH⁻, F⁻, Cl⁻, Br⁻, I⁻, ⅓ PO₄³⁻, ½ SO₄²⁻, R¹⁹COO⁻, R¹⁹SO₃⁻, preferably R³-R⁷ are H or CH₃;
   preferably R⁸ is C₁-C₃₀, or

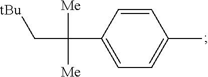

preferably R⁹-R¹² are H or C₁-C₁₀;
preferably R¹³-R¹⁸ are H or CH₃;
n is an integer and can have a value comprised between 1 and 500, preferably n is comprised between 6 and 50, alternatively between 1 and 10; in some specific embodiments, n may be 0. In other words, n is an integer from 0 to 500.
p is an integer and can have a value comprised between 1 and 10, preferably p=2;
q is an integer and can have a value comprised between 1 and 500, preferably q=2-4. W¹ may further be —(CR⁹R¹⁰)OC(O)(ZO)ₙ—R⁸.
   Q⁻ is preferably a pharmaceutically acceptable anion. R¹⁹ is C₁-C₁₀ alkyl group, optionally substituted by one or several halogens. Preferably R¹⁹ is CH₃, CH₃—CH₂— or CF₃—.

In certain embodiments of this aspect of the invention, the compounds can be any one of compounds as set forth in the examples.

In a more specific aspect, the invention relates to a compound of formula (I)

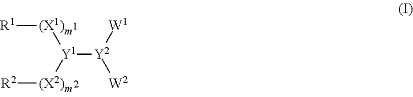

or a salt thereof wherein:
R¹ and R² are independently selected from the groups consisting of linear, unsaturated or saturated, C₈ to C₃₀ alkyl groups eventually interrupted by one or several heteroatoms and eventually substituted by one or several groups selected from C₁-C₃ alkyl groups, halogens, —OH, —OMe, and —CF₃,
X¹ and X² are independently selected from the group consisting of —O—, —OC(O)—, —C(O)O—, —OC(O)O—, —S—, —SS—, —SC(O)—, —OC(S)—, —NR³—, —NR³C(O)—, —C(O)NR³—, —NR³C(S)—, —C(S)NR³—, —OC(O)S—, —OC(S)O—, —SC(O)O—, —OC(S)S—, —SC(O)S—, —SC(S)O—, —SC(S)S—, —OC(O)NR³—, —OC(S)NR³—, —NR³C(S)O—, —NR³C(O)S—, —NR³C(O)NR⁴—, —NR³C(S)NR⁴—, —SC(O)S—, —SC(S)O—, —S(O)—, —S(O)₂—, —O(CR³R⁴)O—, —C(O)O(CR³R⁴)O—, —OC(O)O(CR³R⁴)O—, —P(O)(R³)—, —P(O)(OR³)—, —P(O)(R³)O—, —OP(O)(OR³)—, —OP(O)(R³)O—, —NR³P(O)(R⁴)—, —NR³P(O)(OR⁴)—, —NR³P(O)(R⁴)O—, —OP(O)(OR³)— and —OP(O)(R³)O— wherein R³ and R⁴ are independently H or CH₃,
m¹ and m² are integers independently selected from 0 and 1,
Y¹ and Y² are trivalent connectors selected from the group consisting of N<, —CON<,

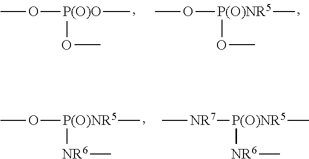

and linear or branched alkyl C¹-C¹⁰ groups, wherein R⁵, R⁶ and R⁷ are independently selected from H and CH₃,
W² is a straight or branched radical comprising from 2 to 20 carbon atoms and at least one functional group selected from ester, carboxylate, —OH, ether, primary, secondary, tertiary or quaternary amine and combinations thereof.
W¹ is a radical having the following formula (II):

-(L)ₛ-(ZO)ₙ—R⁸ wherein
s is 0 or 1,
n is an integer from 0 to 30, preferably from 0 to 22 with the proviso that n is not 0 when s is 0.
L is a divalent connector. When present, L is preferably a C₁-C₂₂ hydrolyzable connector. Preferably, L is —C(R²⁰)(R²¹)—O—C(O)—, —C(R²²)(R²³)—O—C(O)—O— or C(R²⁴)(R²⁵)—O—C(O)—(CH₂)ₚ—C(O)O— wherein R²⁰ to R²⁵ are selected from the group consisting of H and C₁-C₃ alkyl groups, which may be linear, cyclic or branched, and p is an integer between 1 to 10.
More preferably, L is —CH(R²⁰)—O—C(O)—, —CH(R²²)—O—C(O)—O— or CH(R²⁴)—O—C(O)—(CH₂)ₚ—C(O)O— wherein R²⁰, R²², and R²⁴ are selected from the group consisting of H and C₁-C₃ alkyl groups and p is an integer between 1 to 10,
Z is —CH₂CH₂—, —CH₂CH₂CH₂— or —CH₂CH₂CH₂CH₂— and
R⁸ is selected from the group consisting of:
   Unsaturated or saturated linear C₁-C₂₄ alkyl groups, preferably C₅-C₂₄ groups, optionally substituted with one or several groups selected from F, —Cl, —Br, —I, —OH, —OMe, C₁-C₄ alkyl groups and CF₃ and optionally interrupted by an heteroatom.
   An aryl group substituted by one or several C₁-C₁₂ linear or branched alkyl groups, preferably

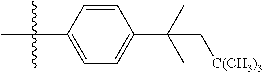 [SMILES: *c1ccc(C(C)(C)CC(C)(C)C)cc1]

As used herein, an heteroatom encompasses NH, O and S.

As used herein, C₁-C₃ alkyl groups encompass methyl, ethyl, propyl and isopropyl.

A C₄ alkyl group encompasses n-butyl, sec-butyl, isobutyl and tert-butyl

“s is 0” means that L is not present. In such a case, W¹ is —(ZO)ₙ—R⁸.

As illustrated above, W¹ moiety may comprise a hydrolyzable connector.

As used herein, a hydrolyzable connector means a linker that can be cleaved under specific pH conditions (acid or basic conditions) or by specific cellular enzymes.

Without to be bound by any theory, the Applicant believes that the presence of a hydrolyzable connector may modulate the transfection activity of the compound and/or make easier its metabolization by the cell by promoting the release of —(ZO)ₙ—R⁸ moiety in the endosome compartment. In some cases, the release of —(ZO)ₙ—R⁸ inside the endosomal compartment may promote the release of active molecule such as a RNA molecule, through the destabilization of endosome membrane, in the cytosol.

Preferably, the hydrolyzable connector is selected so that the stability (namely, the half-life t_{1/2}) of the resulting compound in aqueous media is sufficient to enable the delivery of an active compound into the intracellular compartment without impairing the effective release of the said active molecule in the nucleus and/or the cytosol. The hydrolyzable connector may be also selected so that the compound of the invention may be metabolized by the cell within a reasonable time after transfection.

Notably, as illustrated in the examples of the instant application, when present, L is preferably selected from the group consisting of —C(R²⁰)(R²¹)—O—C(O)—, —C(R²²)(R²³)—O—C(O)—O— or —C(R²⁴)(R²⁵)—O—C(O)—(CH₂)ₚ—C(O)O— wherein R²⁰ to R²⁵ are selected from the group consisting of H and C₁-C₃ alkyl groups, which may be linear, cyclic or branched, and, p is an integer between 1 to 10. More preferably, R²¹, R²³ and R²⁵ are hydrogen. Still more preferably, it is selected from the group consisting of —CH₂—O—C(O)—, —CH₂—O—C(O)—(CH₂)₂—C(O)O— and —CH(R²²)—O—C(O)—O— wherein R²² is —H, —CH₃ or —CH(CH₃)₂. Even more preferably, L is —CH₂—O—C(O)— or —CH(R²²)—O—C(O)—O— wherein R²² is —H, —CH₃ or —CH(CH₃)₂.

As used herein, “Unsaturated C₁-C₂₄ alkyl group” encompass C¹-C²⁴ alkyl groups comprising one or several triple and/or double bonds. Similarly, “Unsaturated C⁵-C²⁴ alkyl group” encompass C⁵-C²⁴ alkyl groups comprising one or several triple and/or double bonds. Preferably, “one or several triple and/or double bonds” encompass 1, 2, 3 and 4 double and/or triple bonds.

“One or several substituents” encompass 1, 2, 3, 4, 5, 6 and 7 substituents.

As used herein, “A C₁-C₂₄ alkyl group interrupted by an heteroatom” means a group of formula —R²⁶—X—R²⁷ wherein X is an heteroatom, preferably selected from —O—, —S— and —NH— and R²⁶ and R²⁷ are linear Cₓ and C_{y} alkyl groups respectively, such that 2≦x+y≦24 with x and y are integers. In addition, “A C₅-C₂₄ alkyl group interrupted by an heteroatom” means a group of formula —R²⁶—X—R²⁷ wherein X is an heteroatom, preferably selected from —O—, —S— and —NH— and R²⁶ and R²⁷ are linear Cₓ and C_{y} alkyl groups respectively, such that 5≦x+y≦24 with x and y are integers.

In some preferred embodiments, R⁸ is a C₁-C₂₄ linear alkyl group, preferably a C₅-C₂₄ linear alkyl group, having one or several (1, 2 or 3) of the following features:
a) R⁸ comprises from 1 to 7 substituents, preferably 1, 2, 3 or 4 substituents, selected from —F, —Cl, —Br, —I, —OH, —OMe, C₁-C₄ alkyl groups and —CF₃, preferably from C₁-C₄ alkyl groups and more preferably from CH₃—, CH₃—CH₂— and —OMe, and/or
b) R⁸ comprises 1, 2, 3 or 4 double and/or triple bond(s), and/or
c) R⁸ is interrupted by a heteroatom, preferably O or NH.

In more preferred embodiments, R⁸ is selected from the group consisting of:
unsubstituted and unsaturated, straight C₁-C₂₄ alkyl groups, preferably C₅-C₂₄ alkyl groups, and
Linear C₁-C₂₄ alkyl groups, preferably C₅-C₂₄ alkyl groups, comprising:
   1 or 2 double or triple bonds and/or
   1, 2, 3 or 4 substituents selected from —F, —Cl, —Br, —I, —OH, —OMe, C₁-C₄ alkyl groups and —CF₃, preferably from CH₃—, CH₃—CH₂— and —O—CH₃.

In other embodiments, R⁸ is selected from the group consisting of:
—(CH₂)ₓCH₃ with x an integer from 0 to 23, preferably from 0 to 16, more preferably from 4 to 14, and
—(CH₂)_{z}—CH═CH—(CH₂)_{y}—CH₃ with x and y are integers such that 2≦y+z≦21. The double bond may be of cis or trans configuration.

Preferably, R⁸ is selected from the group consisting of:
—(CH₂)ₓCH₃ with x an integer from 4 to 23, preferably from 4 to 16, more preferably from 8 to 14, and
—(CH₂)_{z}—CH═CH—(CH₂)_{y}—CH₃ with x and y are integers such that 2≦y+z≦21. The double bond may be of cis or trans configuration.

Examples of appropriate R⁸ group encompass, without being limited to, —(CH₂)₁₀CH₃, —(CH₂)₁₁CH₃, —(CH₂)₄—CH₃, —(CH₂)₁₇—CH₃ and —(CH₂)₈—CH═CH—(CH₂)₈—CH₃.

In some embodiments, R⁸ is a C⁸-C¹⁶ alkyl group, preferably C¹⁰-C¹⁴ alkyl groups such that a C¹² or a C¹¹ alkyl group.

In other embodiments of the invention, R⁸ is

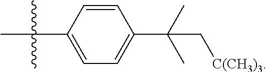 [SMILES: *c1ccc(C(C)(C)CC(C)(C)C)cc1]

In some further embodiments, when R⁸ is

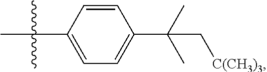 [SMILES: *c1ccc(C(C)(C)CC(C)(C)C)cc1]

### L is not —CH(CH₃)—O—C(O)—(CH₂)₂—C(O)—O—.

In some other embodiments, when R⁸ is

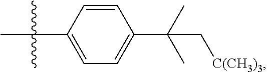 [SMILES: *c1ccc(C(C)(C)CC(C)(C)C)cc1]

the compound of the invention is characterized by the following features:

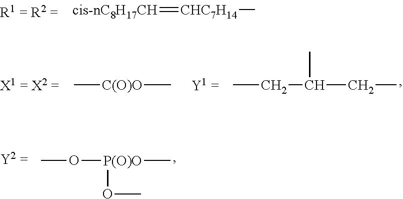

W²=—CH₂—CH₂₋NMe₃⁺Q⁻ with Q⁻ a pharmaceutically acceptable anion, preferably Cl⁻, CF₃SO₃⁻ or CF₃CO₂⁻ and
W¹ is of formula (L)ₛ-(CH₂—CH₂—O)ₙ—R⁸ wherein:
   s is 0 or 1
   n is 9 or 10, and
   L is —CH(CH₃)—O—C(O)—O—, —CH₂—O—C(O)—O—, or —CH₂—O—C(O)—(CH₂)₂—C(O)—O—

The compounds of the invention may be used for promoting in vitro or in vivo delivery of active compounds into cells. The nature of W² may thus vary upon the active molecule to deliver into the cell. When the active molecule is cationic, W² is preferably a group which is negatively charged. In the same way, if the active molecule is anionic, W² is preferably a cationic or a protonable group. For example, if the active molecule to transfect is a nucleic acid, W² may be virtually any cationic or protonable group commonly used in synthetic vectors dedicated to the transfection of nucleic acids.

For example, W² may contain one or several protonable or charged groups such as primary, secondary, tertiary or quaternary amino groups. For example, W² may be an alkylamine, a tetralkylammonium, a straight or branched oligoethylenimine comprising from 2 to 6 monomers, a polyazaalkylamine such as spermine radical, e.g., a radical including a motif such as —(CH₂)₃₋₄NH(CH₂)₃₋₄NH—.

W² may derive from the side-chains of amino acids such as alanine, lysine, arginine and histidine or may derive from a whole amino acid such as serine.

W² is preferably selected from the group consisting of —CH₂—CH(COOH)NH₂ and its salts, —CH₂—CH(OH)—CH₂—OH, straight or branched oligoethylenimine comprising from 2 to 6 monomers, —(Z¹NR¹⁴)_{q}—R¹⁵ and —Z¹NR¹⁶R¹⁷R¹⁸⁺Q⁻ wherein Z¹, being the same or different in each repeat, is —(CH₂)₂—, —(CH₂)₃— or —(CH₂)₄—, q is an integer from 1 to 4, R¹⁴ to R¹⁸ is H or CH₃ and Q⁻ is a pharmaceutically acceptable anion. For example, Q may be selected from the group consisting of OH⁻, F⁻, Cl⁻, Br⁻, I⁻, ⅓ PO₄³⁻, ½ SO₄²⁻, CH₃COO⁻, CF₃COO⁻, and CF₃SO₃⁻.

In some preferred embodiments, Y¹ is a linear or branched alkyl C₁-C₁₀ group, preferably a C₁-C₄ and Y² is selected from the group consisting of

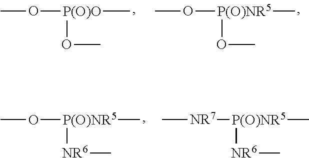

wherein R⁵, R⁶ and R⁷ are independently selected from H and CH₃.

In some embodiments m¹=m²=1 and thus X¹ and X² are both present.

In some preferred embodiments, X¹ and X² are independently selected from the group consisting of —O—, —OC(O)—, —C(O)O—, —OC(O)O—, —OC(S)—, —C(S)—O—, —NH—, —NH—C(O)—, —NH—C(O)—NH— and —C(O)—NH—. X¹ and X² may be identical or distinct. For example, X¹=X²=—C(O)—O—

As mentioned above, R¹ and R² are independently selected from the groups consisting of linear, unsaturated or saturated, C₈ to C₃₀ alkyl groups eventually interrupted by one or several heteroatoms and eventually substituted by one or several groups selected from C₁-C₃ alkyl groups, halogens, —OH, —OMe, —CH₃, and —CF₃.

C₈-C₃₀ alkyl groups encompass C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ alkyl groups. C₈-C₃₀ alkyl groups encompass C₁₂-C₂₄ alkyl groups, C₁₄-C₂₀ alkyl groups and C₁₆-C₁₈ alkyl groups.

As used herein, “an unsaturated alkyl group” comprises one or several unsaturations. Unsaturations encompass double bonds and triple bonds.

In some embodiments, R¹ and R² are independently selected from linear C₁-C₃₀, preferably C₁₂-C₂₄, alkyl groups comprising 0, 1, 2, 3 or 4 unsaturations, the said unsaturation(s) being preferably double bond(s).

When R¹ and/or R² comprise(s) one or several double bonds, the said double bond(s) may be independently in cis or trans configuration. Preferably, the double bond(s) is/are in cis configuration.

An example of C₁₂-C₂₄ alkyl groups is —(CH₂)₇—CH═CH—(CH₂)₇—CH₃.

In some other embodiments, R¹ and/or R² are independently selected from the group consisting of the alkyl chains of naturally-occurring fatty acids. As used herein, the alkyl chain of a fatty acid of formula R—COOH is R— group and thus does not contain the carbon atom of the carboxylate group. For example the alkyl chain of oleic acid (CH₃(CH₂)₇CH═CH(CH₂)₇COOH) is CH₃(CH₂)₇CH═CH(CH₂)₇—

Examples of naturally-occurring fatty acids and corresponding alkyl chain are shown in table 1 hereunder:

TABLE 1
naturally-occurring fatty acid

| Common name | Alkyl chain |
|---|---|
| Myristoleic acid | CH₃(CH₂)₃CH═CH(CH₂)₇₋ |
| Palmitoleic acid | CH₃(CH₂)₅CH═CH(CH₂)₇₋ |
| Sapienic acid | CH₃(CH₂)₈CH═CH(CH₂)₄₋ |
| Oleic acid | CH₃(CH₂)₇CH═CH(CH₂)₇₋ |
| Elaidic acid | CH₃(CH₂)₇CH═CH(CH₂)₇₋ |
| Vaccenic acid | CH₃(CH₂)₅CH═CH(CH₂)₉₋ |
| Linoleic acid | CH₃(CH₂)₄CH═CHCH₂CH═CH(CH₂)₇₋ |
| Linoelaidic acid | CH₃(CH₂)₄CH═CHCH₂CH═CH(CH₂)₇₋ |
| α-Linolenic acid | CH₃CH₂CH═CHCH₂CH═CHCH₂CH═CH(CH₂)₇₋ |
| Arachidonic acid | CH₃(CH₂)₄CH═CHCH₂CH═CHCH₂CH═CHCH₂CH═CH(CH₂)₃₋ |
| Erucic acid | CH₃(CH₂)₇CH═CH(CH₂)₁₁— |
| Caprylic acid | CH₃(CH₂)₆— |
| Capric acid | CH₃(CH₂)₈₋ |
| Lauric acid | CH₃(CH₂)₁₀₋ |
| Myristic acid | CH₃(CH₂)₁₂₋ |
| Palmitic acid | CH₃(CH₂)₁₄₋ |
| Stearic acid | CH₃(CH₂)₁₆₋ |
| Arachidic acid | CH₃(CH₂)₁₈₋ |
| Behenic acid | CH₃(CH₂)₂₀₋ |
| Lignoceric acid | CH₃(CH₂)₂₂₋ |
| Cerotic acid | CH₃(CH₂)₂₄₋ |

In some embodiments R¹ and R² are identical.

In preferred embodiments, the compound of the invention is a compound of formula (I) in wherein:
m¹=m²=1
Y¹ is

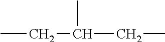

Y² is

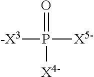

with X³, X⁴ and X⁵ independently selected from —O—, —NH— and NHCH₃—

In such an embodiment, the compound of the invention has thus the following formula (III)

### Wherein:

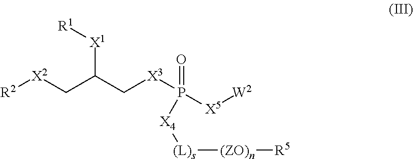 [0160] X³, X⁴ and X⁵ are independently selected from the group consisting of —O—, —NH— and —NCH₃—, preferably, from —O— and —NH— and
   R¹, R², X¹, X², W², L, Z, R⁸, n and s are as previously described in the specification for the general formula (I) and its various embodiments.

In a particular embodiment, the invention relates to a compound of formula (III) in which X¹=X²=—C(O)O— and, X³=X⁴=X⁵=O.

In other words, the said compound is a compound of formula (I) as defined above wherein
m¹=m²=1
X¹=X²=—C(O)O—
Y¹ is

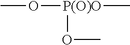

and
Y² is

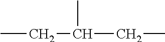

In such an embodiment, the compound derives from a phospholipid, i.e. the compound is a conjugate of a phospholipid, more precisely, a O-substituted phospholipid compound.

Accordingly, another aspect of the invention is a compound of the phospholipid-type having the following formula (IV):

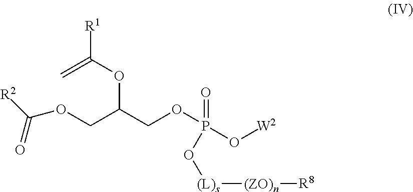

wherein R¹, R², L, s, z and R⁸ are as previously described in the specification for the general formula (I) and its various embodiments.

In some particular embodiments, the compound of the invention is a compound of formula (IV) wherein:
R¹ and R² are independently selected from the group consisting of linear, unsaturated or saturated, C₈ to C₃₀, preferably C₁₂ to C₂₄, alkyl groups eventually interrupted by a heteroatom and eventually substituted by one or several groups selected from C₁-C₃ alkyl groups, halogens, —OH, —OMe, and —CF₃, preferably C₁-C₃ alkyl groups,
W² is a straight or branched radical comprising from 2 to 20 carbon atoms and at least one functional group selected from ester, carboxylate, —OH, ether, primary, secondary, tertiary or quaternary amine and combinations thereof. Preferably, W² is selected from the group consisting of —CH₂—CH₂—C(COOH)NH₂ and its salts, a straight or branched oligoethylenimine comprising from 2 to 6 monomers, —CH₂—CH(OH)—CH₂—OH, —(Z¹NR¹⁴)_{q}—R¹⁵ and —Z¹NR¹⁶R¹⁷R¹⁸⁺Q⁻ wherein Z¹ is the same or different and is selected from the group consisting of —(CH₂)₂—, —(CH₂)₃— or —(CH₂)₄—, q is an integer from 1 to 4, R¹⁴ to R¹⁸ are H or CH₃, and Q⁻ is a pharmaceutically acceptable anion,
s is 0 or 1,
n is an integer from 0 to 30, preferably from 0 to 22 with the proviso that n is not 0 when s is 0. When s is 0, n is preferably from 2 to 12, more preferably from 2 to 9,
L is —CH(R²⁰)—O—C(O)—, —CH(R²²)—O—C(O)—O— or —CH(R²⁴)—O—C(O)—(CH₂)ₚ—C(O)O— wherein
   R²⁰, R²², and R²⁴ are selected from the group consisting of H and C¹-C³ alkyl groups and
   p is an integer between 1 to 10, preferably 2.
Z is —CH₂CH₂—, —CH₂CH₂CH₂— or —CH₂CH₂CH₂CH₂—, preferably —CH₂—CH₂— and
R⁸ is selected from the group consisting of
   Unsaturated or saturated linear C₁-C₂₄ alkyl groups, preferably C₅-C₂₄ alkyl groups, optionally substituted with one or several groups selected from —F, —Cl, —Br, —I, —OH, —OMe, C₁-C₄ alkyl groups and —CF₃ and optionally interrupted by an heteroatom.
   aryl groups substituted by one or several C₁-C₁₂ linear or branched alkyl groups, preferably

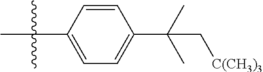 [SMILES: *c1ccc(C(C)(C)CC(C)(C)C)cc1]

Accordingly, the compound of the invention may be a conjugate of a phospholipid in which the phosphoric acid residue is covalently linked to -(L)ₛ-(ZO)ₙ—R⁸ radical so as to form a phosphotriester.

As used herein, a phospholipid is an ester of glycerol with two molecules of fatty acids (the same or different) and phosphoric acid, wherein the phosphoric acid residue is in turn bonded to a hydrophilic group. The phospholipid may be a naturally occurring phospholipid such as lecithins from soya bean or egg yolk as well as a synthetic phospholipid.

In some embodiments, W² is selected from the group of —CH₂—CH₂—N(CH₃)₃⁺Q⁻, —CH₂—CH₂—NH₂ and its salts, —CH₂—CH(COOH)NH₂ and its salts, and —CH₂—CH(OH)—CH₂—OH. In such a case, the compound of the invention is a conjugate of a phosphatidylcholine, that of a phosphatidylethanolamine, that of a phosphatidylserine, or that of a phosphatidylglycerol, respectively.

Phosphatidylcholines encompass, without being limited to, dilauroylphosphatidylcholine (“DLPC”), dimyristoylphosphatidylcholine (“DMPC”), dipalmitoylphosphatidylcholine (“DPPC”), diarachidoylphosphatidylcholine (“DAPC”), distearoylphosphatidylcholine (“DSPC”), 1-myristoyl-2-palmitoylphosphatidylcholine (“MPPC”), 1-palmitoyl-2-myristoylphosphatidylcholine (“PMPC”), 1-palmitoyl-2-stearoylphosphatidylcholine (“PSPC”), 1-stearoyl-2-palmitoylphosphatidylcholine (“SPPC”), dioleoylphosphatidylycholine (“DOPC”), 1-palmitoyl-2-oleoylphosphatidylcholine (“POPC”), 1-stearoyl-2-oleoylphosphatidylcholine (“SOPC”), 1-myristoyl-2-oleoylphosphatidylcholine (“MOPC”), and 1-lauroyl-2-oleoylphosphatidylcholine (“LOPC”).

Examples of phosphatidylglycerol encompass, without to be limited to, dilauroyl-phosphatidylglycerol (“DLPG”) and its alkali metal salts, diarachidoylphosphatidylglycerol (“DAPG”) and its alkali metal salts, dimyristoylphosphatidylglycerol (“DMPG”) and its alkali metal salts, dipalmitoyl-phosphatidylglycerol (“DPPG”) and its alkali metal salts, distearolyphosphatidylglycerol (“DSPG”) and its alkali metal salts, dioleoylphosphatidylglycerol (“DOPG”) and its alkali metal salts, 1-palmitoyl-2-oleoylphosphatidylglycerol (“POPG”) and its alkali metal salts, 1-stearoyl-2-oleoylphosphatidylglycerol (“SOPG”) and its alkali metal salts, 1-myristoyl-2-oleoylphosphatidylglycerol (“MOPG”) and its alkali metal salts, 1-lauroyl-2-oleoylphosphatidylglycerol (“LOPG”) and its alkali metal salts.

Examples of phosphatidylethanolamine encompass, without to be limited to, dipalmitoyl phosphatidylethanolamine (“DPPE”) and distearoyl phosphatidylethanolamine (“DSPE”), dilauroyl-phosphatidylethanolamine (“DLPE”), dimyristoylphosphatidylethanolamine (“DMPE”), dioleoylphosphatidylethanolamine (“DOPE”), diarachidoylphosphatidylethanolamine (“DAPE”), 1-myristoyl-2-palmitoylphosphatidylethanolamine (“MPPE”), 1-palmitoyl-2-myristoylphosphatidylethanolamine (“PMPE”), 1-palmitoyl-2-stearoylphosphatidylethanolamine (“PSPE”), 1-stearoyl-2-palmitoyl-phosphatidylethanolamine (“SPPE”), 1-palmitoyl-2-oleoylphosphatidylethanolamine (“POPE”), 1-stearoyl-2-oleoylphosphatidylethanolamine (“SOPE”), 1-myristoyl-2-oleoylphosphatidylethanolannine (“MOPE”), and 1-lauroyl-2-oleoylphosphatidylethanolannine (“LOPE”).

Examples of phosphatidylserine encompass dilauroylphosphatidylserine (“DLPS”), dimyristoylphosphatidylserine (“DMPS”), dipalmitoylphosphatidylserine (“DPPS”), distearoylphosphatidylserine (“DSPS”), dioleoylphosphatidylserine (“DOPS”), diarachidoylphosphatidylserine (“DAPS”), 1-myristoyl-2-palmitoylphosphatidylserine (“MPPS”), 1-palmitoyl-2-myristoylphosphatidylserine (“PMPS”), 1-palmitoyl-2-stearoylphosphatidylserine (“PSPS”), 1-stearoyl-2-palmitoyl-phosphatidylserine (“SPPS”), 1-palmitoyl-2-oleoylphosphatidylserine (“POPS”), 1-stearoyl-2-oleoylphosphatidylserine (“SOPS”), 1-myristoyl-2-oleoylphosphatidylserine (“MOPS”), and 1-lauroyl-2-oleoylphosphatidylserine (“LOPS”).

In some embodiments, in particular when the compound is to be used for the transfection of a nucleic acid, W² is preferably —CH₂—CH₂—N(CH₃)₃⁺Q⁻. In such a case, the compound is a conjugate of a phosphatidylcholine, which generally displays low cell toxicity. This may be explained by the fact that phosphatidylcholines are endogeneous membrane phospholipids and their conjugates may be slowly metabolized by intracellular phospholipases. In order to further improve intracellular metabolization and thus decrease cell toxicity, the compound of the invention may further comprise a hydrolyzable linker L (which implies that s=1).

In some further embodiments, the invention relates to a compound of formula (IV) in which:
W² is —CH₂—CH₂—N(CH₃)₃⁺Q⁻ with Q⁻ pharmaceutically acceptable anion.
s=1 and
L is selected from the group consisting of: —CH₂—O—C(O)—(CH₂)₂—C(O)—O—, —CH₂—O—C(O)— and CH(R²²)—O—C(O)—O— with R²²=H, CH₃ or —CH(CH₃)₂.

In some specific embodiments, the compound of the invention is a compound of formula (IV) as defined above which further has one or several (1, 2, 3, 4 or 5) of the following features:
i) W² is —CH₂—CH₂—N(CH₃)₃⁺Q⁻ with Q⁻ is a pharmaceutically acceptable anion,
ii) R² and R¹ are independently selected from the group consisting of saturated, unsubstituted C₁₂-C₂₄, preferably C₁₄-C₂₀ alkyl chain and unsaturated, unsubstituted, C₁₂₋₂₄, preferably C₁₄-C₂₀ alkyl chains having 1, 2, 3 or 4 double bonds, for example, the corresponding alkyl chains shown in table 1 hereabove,
iii) Z is —CH₂—CH₂—,
iv) R⁸ is independently selected from the group consisting of:
   —(CH₂)ₓCH₃ with x an integer from 4 to 24, preferably from 4 to 16, more preferably from 8 to 14,
   —(CH₂)_{y}—CH═CH—(CH₂)_{z}—CH₃ with x and y are integers such that 2≦y+z≦21, and

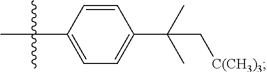 [SMILES: *c1ccc(C(C)(C)CC(C)(C)C)cc1]

and
v) -(L)ₛ-(ZO)ₙ is such that:
   when s=0, n is an integer from 2 to 22, preferably from 2 to 10, more preferably from 3 to 9 and
   when s=1, n is an integer from 0 to 10 and L is selected from the group consisting of: —CH₂—O—C(O)—(CH₂)₂—C(O)—O—, —CH₂—OC(O)— and —CH(R²²)—OC(O)—O— with R²²═H, CH₃ or —CH(CH₃)₂.

For example, this compound may have one of the following combinations of features:
Features iii) and iv);
Features ii), iii), and iv);
Features i), iii), and iv);
Features i), ii), iii) and iv);
Features iii), iv) and v);
Features i), iii), iv) and v);
Features ii), iii), iv) and v); and
Features i), ii), iii), iv) and v)

In some other specific embodiments, the compound of the invention is a compound of formula (IV) wherein
R⁸ is

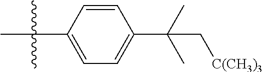 [SMILES: *c1ccc(C(C)(C)CC(C)(C)C)cc1]

n is an integer from 6 to 20, preferably from 6 to 10, more preferably from 7 to 9,
Z is —CH₂—CH₂— and
s=0 or s=1 with L selected from the group consisting of: CH₂—O—C(O)—(CH₂)₂—C(O)—O—, —CH(CH₃)—O—C(O)O— and —CH₂—O—C(O)O—

In such an embodiment, the compound may further displays features i) and/or ii) as described hereabove. For example, R¹═R²═CH₃—(CH₂)₇—CH═CH—(CH₂)₇— and W² is —CH₂—CH₂—N(CH₃)₃⁺Q-.

In some further embodiments, the compound of the invention is a compound of formula (IV) wherein:
R₈ is a linear, saturated or unsaturated, unsubstituted, C₁-C₂₅, preferably C₅-C₂₅, more preferably C₅-C₁₉, alkyl group and more preferably —(CH₂)₁₁CH₃, —(CH₂)₁₀CH₃ or (CH₂)₈—CH═CH—(CH₂)₇—CH₃,
s=0,
Z is —CH₂—CH₂— and,
n is an integer from 1 to 22, preferably from 2 to 22, more preferably from 2 to 8, and still more preferably from 3 to 7, such as 4.

In such an embodiment, the compound may further displays features i) and/or ii) as described hereabove. For example, R¹═R²═CH₃—(CH₂)₇—CH═CH—(CH₂)₇— and W² is —CH₂—CH₂—N(CH₃)₃⁺Q-.

In another embodiment, the compound of the invention is a compound of formula (IV) wherein:
R₈ is a linear, saturated or unsaturated, unsubstituted, C₁-C₂₅, preferably C₅-C₂₅, more preferably C₆-C₁₉, alkyl group and more preferably —(CH₂)₁₁CH₃, —(CH₂)₁₀CH₃ or —(CH₂)₈—CH═CH—(CH₂)₇—CH₃,
s=1
Z is —CH₂—CH₂— and,
n is an integer from 0 to 22, preferably from 0 to 8, and more preferably from 3 to 7, such as 4, and
L selected from the group consisting of: —CH₂—O—C(O)—, —CH₂—O—C(O)—O—, —CH(CH₃)—O—C(O)—O—, —CH(iPr)—O—C(O)—O— and CH₂—O—C(O)—CH₂—CH₂—C(O)—O—

In such an embodiment, the compound may further display features i) and/or ii) as described hereabove. For example, the compound may further be characterized in that R¹═R²═CH₃—(CH₂)₇—CH═CH—(CH₂)₇— and W² is —CH₂—CH₂—N(CH₃)₃⁺Q-. Moreover, R⁸ is preferably —(CH₂)₁₁CH₃, or —(CH₂)₁₀CH₃.

Several compounds according to the invention are provided hereunder in the Examples.

In some other embodiments, the invention relates to a compound of formula (V)

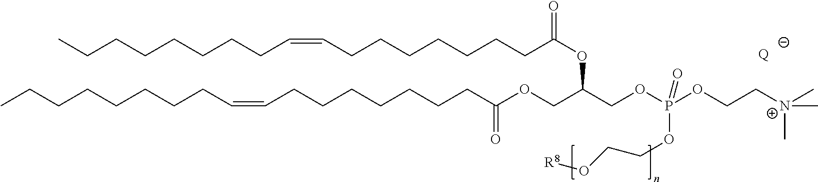

(V), wherein Q⁻ is a pharmaceutically acceptable anion and characterized by a combination of features selected from the group consisting of:
n=1 and R⁸ is CH₃—(CH₂)₁₁ (such as compound PP531 as described in the example part),
n=about 4 (e.g., between 3 and 5), preferably 4, and R⁸ is CH₃—(CH₂)₁₁ (e.g. compound PP415),
n=about 7 (e.g., between 5 and 9), preferably 7, and R⁸ is CH₃—(CH₂)₁₁ (e.g. compound PP418),
n=3 and R⁸ is CH₃—(CH₂)₁₁ (e.g. compound PP529),
n=about 22 (e.g., between 18 and 26), preferably 22, and R⁸ is CH₃—(CH₂)₁₁ (e.g. compound PP427),
n=about 7 (e.g., between 5 and 9), preferably 7, and R⁸ is CH₃—(CH₂)₁₇ (e.g. compound PP426),
n=about 7 (e.g., between 5 and 9), preferably 7, and R⁸ is cis CH₃—(CH₂)₈—CH═CH—(CH₂)₈ (e.g. compound PP522), and
n=about 7 (e.g., between 5 and 9), preferably 7, and R⁸ is CH₃—(CH₂)₅ (e.g. compound PP419).

Preferably, the invention relates to one or several compounds of formula (V) characterized by a combination of features selected from the group consisting of:
n=about 4 (e.g., between 3 and 5), preferably 4, and R⁸ is CH₃—(CH₂)₁₁,
n=3 and R⁸ is CH₃—(CH₂)₁₁, and
n=about 7 (e.g., between 5 and 9), preferably 7, and R⁸ is CH₃—(CH₂)₁₁.

In some other embodiments, the invention relates to a compound of formula (VI)

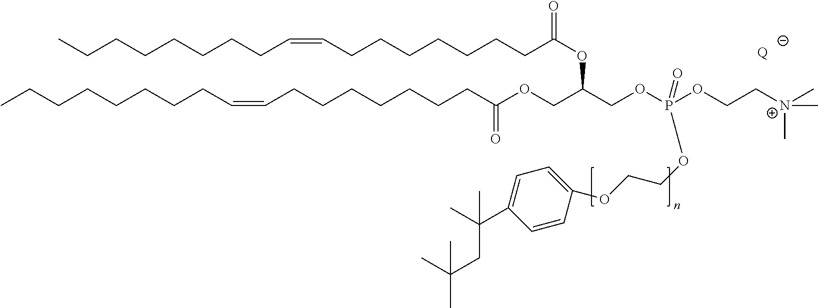

(VI) wherein Q⁻ is a pharmaceutically acceptable anion and n is about 7 (e.g., between 5 and 9), preferably 7 (e.g. in compound PP507) or about 20 (e.g., between 18 and 22), preferably 20 (e.g. in compound PP430).
In some further embodiments, the compound of the invention is of formula (VII):

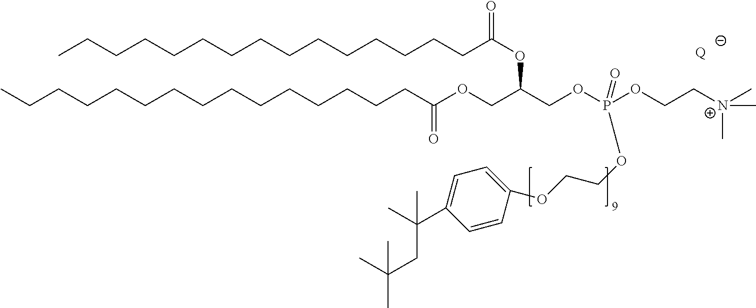

wherein Q⁻ is a pharmaceutically acceptable anion. An example is compound PP431.

In some alternate embodiments, the invention relates to a compound of formula (VIII)

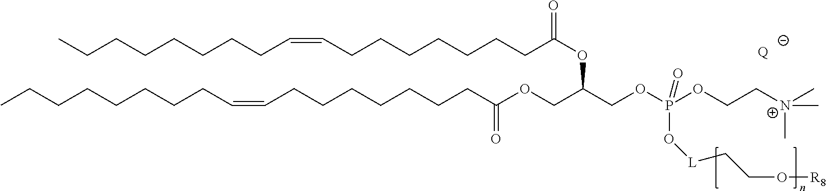

wherein Q⁻ is a pharmaceutically acceptable anion and characterized by a combination of features selected from the group consisting of:
   n=0, R⁸ is —(CH₂)₁₀—CH₃ and L is —CH₂—O—C(O)— (e.g. PP94),
   n=0, R⁸ is —(CH₂)₁₀—CH₃ and L is —CH(CH₃)—O—C(O)— (e.g. in PP140),
   n=0, R⁸ is (CH₂)₄—CH₃ and L is —CH₂—O—C(O)— (e.g. in PP138),
   n=0, R⁸ is (CH₂)₄—CH₃ and L is —CH(CH₃)—O—C(O)— (e.g. in PP189),
   n=0, R⁸ is (CH₂)₁₁₋CH₃ and L is —CH₂—O—C(O)—O— (e.g. PP91),
   n=0, R⁸ is (CH₂)₁₁₋CH₃ and L is —CH(CH₃)—O—C(O)—O— (e.g. in PP120),
   n=0, R⁸ is (CH₂)₁₁₋CH₃ and L is —CH(iPr)—O—C(O)—O— (e.g. in PP178),
   n=0, R⁸ is (CH₂)₁₁₋CH₃ and L is —CH₂—O—C(O)—(CH₂)₂C(O)O— (e.g. in PP93),
   n=about 4 (e.g., between 3 and 5), preferably 4, R⁸ is (CH₂)₁₁₋CH₃ and L is —CH₂—O—C(O)—O— (e.g. in MR22),
   n=about 4 (e.g., between 3 and 5), preferably 4, R⁸ is (CH₂)₁₁₋CH₃ and L is —CH(Me)-O—C(O)—O— (e.g. in MR25),
   n=about 4 (e.g., between 3 and 5), preferably 4, R⁸ is (CH₂)₁₁₋CH₃ and L is —CH(iPr)—O—C(O)—O— (e.g. in MR26), and
   n=about 4 (e.g., between 3 and 5), preferably 4, R⁸ is (CH₂)₁₁₋CH₃ and L is —CH₂—O—C(O)—(CH₂)₂C(O)O— (e.g. in MR28).

Preferably, the invention relates to a compound of formula (VIII) by a combination of features selected from the group consisting of:
n=0, R⁸ is —(CH₂)₁₀—CH₃ and L is —CH₂—O—C(O)— (e.g. PP94),
n=0, R⁸ is (CH₂)₄—CH₃ and L is —CH₂—O—C(O)— (e.g. in PP138),
n=0, R⁸ is (CH₂)₁₁₋CH₃ and L is —CH(CH₃)—O—C(O)—O— (e.g. in PP120),
n=0, R⁸ is (CH₂)₁₁₋CH₃ and L is —CH(iPr)—O—C(O)—O— (e.g. in PP178),
n=about 4 (e.g., between 3 and 5), preferably 4, R⁸ is (CH₂)₁₁₋CH₃ and L is —CH₂—O—C(O)—O— (e.g. in MR22),
n=about 4 (e.g., between 3 and 5), preferably 4, R⁸ is (CH₂)₁₁₋CH₃ and L is —CH₂—O—C(O)—(CH₂)₂C(O)O— (e.g. in MR28), and
n=about 4 (e.g., between 3 and 5), preferably 4, R⁸ is (CH₂)₁₁₋CH₃ and L is —CH(iPr)—O—C(O)—O— (e.g. in MR26).

In some alternate embodiments, the invention relates to a compound of formula (IX)

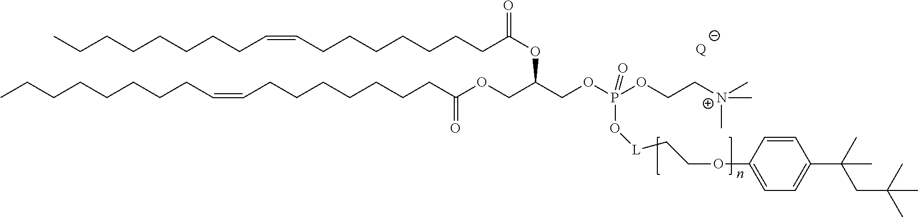

wherein Q⁻ is a pharmaceutically acceptable anion and characterized by a combination of features selected from the group consisting of:
   n=about 9 (e.g., between 7 and 11), preferably 9, and L=—CH₂—O—C(O)O—,
   n=about 10 (e.g., between 8 and 12), preferably 10, and L=—CH₂—O—C(O)O—,
   n=about 9 (e.g., between 7 and 11), preferably 9, and L=—CH(Me)-O—C(O)O—,
   n=about 10 (e.g., between 8 and 12), preferably 10, and L=—CH(Me)-O—C(O)O—,
   n=about 9 (e.g., between 7 and 11), preferably 9, and L=CH₂—O—C(O)—(CH₂)₂C(O)O—, and
   n=about 10 (e.g., between 8 and 12), preferably 10, and L=—CH₂—O—C(O)—(CH₂)₂C(O)O—.

In a particular embodiment, the present invention relates to a mixture of compounds of the invention presenting varying value for n. Indeed, for n greater than 3, the compounds are rather obtained as a mixture of compounds having a means value of n. In particular, when it is referred to a compound having n being about 4, the mixture includes compounds having a means value of n equal to 4.

### II. Supramolecular Complex, Compositions and Uses of the Compounds According to the Invention

The compounds of the invention are able to self-assemble—alone or in the presence of other molecules—into supramolecular complex in aqueous media.

Accordingly, another object to the invention is a supramolecular complex comprising one or several compounds according to the invention. As used herein, a supramolecular complex (or a supramolecular assembly) refers to a complex of molecules held together by noncovalent bonds. In some embodiments, the supramolecular complex further comprises a molecule of interest. The molecule of interest is typically an active compound (namely an active ingredient or a pharmaceutically active compound) to be delivered in cell. Active compounds of interest are described further below. The said supramolecular complex may be a liposome which encapsulates the active compound. The interactions between the constituents of the supramolecular complex are preferably non-covalent interactions. As illustrated in the examples, the compounds of the invention, preferably the compounds of formula (IV) such as phosphatidylcholine conjugates, electrostatically interact with DNA or RNA and form supramolecular complexes. The resulting supramolecular complex has generally a size lower than 1 μm, typically in the range of 100-800 nm.

Accordingly, a supramolecular complex according to the invention preferably comprises one or several phospholipid conjugates of formula (IV) and an active compound selected from nucleic acids such as DNA and RNA. Such a supramolecular complex is usually called lipoplex.

When the supramolecular complex comprises a RNA and a compound of the invention, the charge ratio of the said compound to RNA is from 1 to 100.

When the supramolecular complex comprises a DNA and a compound of the invention, the charge ratio of the said compound to DNA is from 0.5 to 10.

In some embodiments, the supramolecular complex may comprise an additional compound which may bear a moiety enabling to target specific cells such as moieties derived from ligands of membrane receptors.

In a second aspect, the invention pertains to a composition comprising a compound according to the first aspect, namely a compound as defined in part I. hereabove or a supramolecular complex according to the invention, and a carrier.

In certain embodiments, the composition can be a liposomal or lipid formulation comprising one or more compound according to the first aspect of the invention. In an embodiment the composition comprises a further constituent.

In a third aspect, the present invention pertains to a pharmaceutical composition comprising a compound according to the first aspect (namely a compound as defined in part I. hereabove) and a pharmaceutically active compound and preferably a pharmaceutically acceptable carrier. In an embodiment of the hereabove aspects the pharmaceutically active compound and/or the further constituent is selected from the group comprising vectors, low molecular weight drugs, drugs, pharmaceutical compounds, peptides, proteins, oligonucleotides, polynucleotides and nucleic acids.

As used herein, a pharmaceutically active compound refers to a compound which exhibits a biological activity when administered to a living being, preferably an animal. Preferably, the said compound may be used for preventing or treating a disease or a physiological disorder.

As used herein, low-molecular weight drug refers to a drug with a molecular weight of less than 10 kDa, preferably less than 5 kDa.

Examples of such drug include, but are not limited to, bisphosphonate compounds such as Etidronate, Clodronate, Tiludronate, Pamidronate, Neridronate, Olpadronate, Alendronate, Ibandronate, Risedronate or Zoledronate; antitumoral drugs such as taxol, docetaxel, doxorubicin and the like. In some embodiments, the pharmaceutically active compound (also called “active compound”) is an antitumoral.

In an embodiment of the hereabove aspects, the vector is a pharmaceutically acceptable vector, preferably a cationic lipid or a cationic polymer.

In an embodiment of the second and third aspect the low molecular weight drug is a non-polymeric bioactive compound.

In an embodiment of the hereabove aspects, the protein is an antibody, preferably a monoclonal antibody.

As used herein, a nucleic acid may be a single or double stranded molecule of at least 5 nucleotides in length, preferably from 5 to 10,000 nucleotides in length, more preferably from 5 to 200 nucleotides in length, even more preferably from 5 to 50 nucleotides in length. The nucleic acid may be linear or circular such as plasmid. The nucleic acid may comprise chemically modified nucleotides. The said chemical modifications may enable to stabilize the nucleic acid against degradation or to increase its affinity for its biological target. The nucleic acid may contain deoxyribonucleotides, ribonucleotides or nucleotids analogs (Verma and Eckstein, 1998) and inter-nucleotide linkages such as methylphosphonate, morpholino phosphorodiamidate, phosphorothioate and amide.

The nucleic acid may be selected from the group consisting of small interfering RNA (siRNA), double-stranded RNA (dsRNA), double-stranded DNA (dsDNA), single-stranded RNA (ssRNA), single-stranded DNA (ssDNA), micro-RNA (miRNA), short hairpin RNA (shRNA), short hairpin DNA (shDNA) and DNA-RNA duplex.

In an embodiment of the hereabove aspects, the nucleic acid is selected from the group comprising DNA, RNA, PNA and LNA.

In an embodiment of the hereabove aspects, the nucleic acid is a functional nucleic acid, whereby preferably the functional nucleic acid is selected from the group comprising RNAi, siRNA, siNA, antisense nucleic acid, ribozymes, aptamers and spiegelmers.

The charge ratio between the compound of the invention and the active molecule may be from 0.5 to 5000, preferably from 0.5 to 1000. In a particular embodiment, when the molecule is a siRNA, the charge ratio is from 2 to 500, preferably from 5 to 200 and more preferably from 10 to 100. In another embodiment, when the active molecule is a plasmid DNA, the charge ratio of the compound of the invention to the pDNA may be from 0.5 to 500, preferably from 0.5 to 50, more preferably from 0.5 to 5.

In a fourth aspect, the subject invention pertains to the use of a compound according to the first aspect or a composition according to the second or third aspect, for the manufacture of a medicament.

The said compound is preferably used as a transferring or a transfecting agent.

In an embodiment of the fourth aspect, the medicament is for administering the nucleic acid to a cell, preferably a mammalian cell and more preferably a human cell.

In an embodiment of the fourth aspect the medicament is for systemic administration.

In an embodiment of the fourth aspect the medicament is for local administration.

In a fifth aspect, the subject invention pertains to the use of a compound, a supramolecular complex or a composition according to the invention as a transferring agent.

As used herein a transferring agent means a compound able to promote the delivery of a molecule of interest into a cell. The said molecule may be delivered into the cytosol of a cell, the nucleus of a cell or an organelle of the cell.

Accordingly, in an embodiment of the fifth aspect the transferring agent transfers a pharmaceutically active component and/or a further constituent into a cell, preferably a mammalian cell and more preferably a human cell. The said use may be ex vivo, in vivo or in vitro.

In a sixth aspect, the subject invention pertains to a method for transferring a pharmaceutically active compound and/or a further constituent into a cell or across a membrane, preferably a cell membrane, comprising the following steps:
providing the cell or the membrane;
providing a compound according to any of the first aspect;
providing the pharmaceutically active compound and/or the further constituent; and
contacting the cell or the membrane with the pharmaceutically active compound and/or the further constituent, and the compound according to the first aspect.

In a seventh aspect, the subject invention pertains to a method for transferring a pharmaceutically active compound and/or a further constituent into a cell or across a membrane, preferably a cell membrane, providing the following steps:
providing the cell or the membrane;
providing a composition according to the second or third aspect; and
contacting the cell or the membrane with the composition according to the second or third aspect.

The methods of the sixth or the seventh aspect may be in vivo, in vitro or ex vivo.

The cell may be an isolated cell or a cell belonging to a tissue.

In a further aspect, the invention relates to an in vitro or ex vivo method for delivering a molecule of interest to a cell, said method comprising contacting a pharmaceutical composition or a supramolecular complex according to the present invention.

In an eighth aspect, the subject invention pertains to the use of a compound according to the first aspect or a composition according to the second or third aspect for systemic or local administration, preferably systemic or local administration to a vertebrate.

In an embodiment of the eighth aspect, the vertebrate is a mammal, more preferably a mammal selected from the group comprising mouse, rat, guinea pig, cat, dog, monkey and man.

It is to be understood that the compounds according to the present invention are preferably cationic lipids and more preferably O-conjugated phosphatidylcholine phospholipids. More preferably, any amine group present in the compounds according to the present invention is present in a charged or protonatable form. Typically, any positive charge of the compound according to the present invention is compensated by the presence of an anion. Such anion (called Q⁻ in above part I) can be a monovalent or polyvalent anion. Preferred anions are halides, acetate and trifluoroacetate. Halides as used herein are preferably fluorides, chlorides, iodides and bromides. Most preferred are chlorides. Upon association of the cationic lipid and the biologically active compound to be transferred into a cell, the halide anion is replaced by the biologically active compound which preferably exhibits one or several negative charges, although it has to be acknowledged that the overall charge of the biologically active compound is not necessarily negative.

The compounds according to the present invention can form a composition or be part of a composition, whereby such composition comprises a carrier. In such compositions, the compounds according to the present invention are also referred to as the conjugate and/or compound. Such carrier is preferably a liquid carrier. Preferred liquid carriers are aqueous carriers and non-aqueous carriers. Preferred aqueous carriers are water, aqueous buffer systems, more preferably buffer systems having a physiological buffer strength and physiological salt concentration(s). Preferred non-aqueous carriers are solvents, preferably organic solvents such as ethanol, tert.-butanol. Without wishing to be bound by any theory, any water miscible organic solvent can, in principle, be used. It is to be acknowledged that the composition can thus be present as or form liposomes.

It is to be acknowledged that the composition according to the present invention in its various embodiments may also be used as a pharmaceutical composition. In the latter case, the pharmaceutical composition comprises a pharmaceutically active compound and optionally a pharmaceutically acceptable carrier. Such pharmaceutically acceptable carrier may, preferably, be selected from the group of carrier as defined herein in connection with the composition according to the present invention. It will be understood by those skilled in the art that any composition as described herein may, in principle, be also used as a pharmaceutical composition provided that its ingredients and any combination thereof is pharmaceutically acceptable. A pharmaceutical composition comprises a pharmaceutically active compound. Such pharmaceutically active compound can be the same as the further constituent of the composition according to the present invention which is preferably any biologically active compound, more preferably any biologically active compound as disclosed herein. The further constituent, pharmaceutically active compound and/or biologically active compound are preferably selected from the group comprising vectors, low molecular weight drugs or other pharmaceutical compounds, peptides, proteins, oligonucleotides, polynucleotides and nucleic acids.

A peptide as preferably used herein is any polymer consisting of at least two amino acids which are covalently linked to each other, preferably through a peptide bond. More preferably, a peptide consists of two to ten amino acids. A particularly preferred embodiment of the peptide is an oligopeptide which even more preferably comprises from about 10 to about 100 amino acids. Proteins as preferably used herein are polymers consisting of a plurality of amino acids which are covalently linked to each other. Preferably such proteins comprise about at least 100 amino acids or amino acid residues.

A preferred protein which may be used in connection with the cationic lipid and the composition according to the present invention, is any antibody, preferably any monoclonal antibody. Particularly preferred biologically active compounds, i.e. pharmaceutically active compounds and such further constituent as used in connection with the composition according to the present invention are nucleic acids. Such nucleic acids can be either DNA, RNA, PNA or any mixture thereof. More preferably, the nucleic acid is a functional nucleic acid. A functional nucleic acid as preferably used herein is a nucleic acid which is not a nucleic acid coding for a peptide and protein, respectively. Preferred functional nucleic acids are siRNA, siNA, RNAi, antisense-nucleic acids, ribozymes, aptamers and spiegelmers which are all known in the art.

siRNA are small interfering RNA as, for example, described in international patent application PGT/EP03/08666. These molecules typically consist of a double-stranded RNA structure which comprises between 15 to 25, preferably 18 to 23 nucleotide pairs which are base-pairing to each other, i.e. are essentially complementary to each other, typically mediated by Watson-Crick base-pairing. One strand of this double-stranded RNA molecule is essentially complementary to a target nucleic acid, preferably a mRNA, whereas the second strand of said double-stranded RNA molecule is essentially identical to a stretch of said target nucleic acid. The siRNA molecule may be flanked on each side and each stretch, respectively, by a number of additional oligonucleotides which, however, do not necessarily have to base-pair to each other.

RNAi has essentially the same design as siRNA, however, the molecules are significantly longer compared to siRNA. RNAi molecules typically comprise 50 or more nucleotides and base pairs, respectively.

A further class of functional nucleic acids which are active based on the same mode of action as siRNA and RNAi is siNA. siNA is, e.g., described in international patent application PCT/EP03/074654. More particularly, siNA corresponds to siRNA, whereby the siNA molecule does not comprise any ribonucleotides.

Antisense nucleic acids, as preferably used herein, are oligonucleotides which hybridise based on base complementarity with a target RNA, preferably mRNA, thereby activating RNaseH. RNaseH is activated by both phosphodiester and phosphothioate-coupled DNA. Phosphodiester-coupled DNA, however, is rapidly degraded by cellular nucleases with exception of phosphothioate-coupled DNA. Antisense polynucleotides are thus effective only as DNA-RNA hybrid complexes. Preferred lengths of antisense nucleic acids range from 16 to 23 nucleotides. Examples for this kind of antisense oligonucleotides are described, among others, in U.S. Pat. No. 5,849,902 and U.S. Pat. No. 5,989,912.

A further group of functional nucleic acids are ribozymes which are catalytically active nucleic acids preferably consisting of RNA which basically comprise two moieties. The first moiety shows a catalytic activity, whereas the second moiety is responsible for the specific interaction with the target nucleic acid. Upon interaction between the target nucleic acid and the said moiety of the ribozyme, typically by hybridisation and Watson-Crick base-pairing of essentially complementary stretches of bases on the two hybridising strands, the catalytically active moiety may become active which means that it cleaves, either intramolecularly or intermolecularly, the target nucleic acid in case the catalytic activity of the ribozyme is a phosphodiesterase activity. Ribozymes, the use and design principles are known to the ones skilled in the art and, for example, described in Doherty and Doudna (Annu. Ref. Biophys. Biomolstruct. 2000; 30: 457-75).

A still further group of functional nucleic acids are aptamers. Aptamers are D-nucleic acids which are either single-stranded or double-stranded and which specifically interact with a target molecule. The manufacture or selection of aptamers is, e.g., described in European patent EP 0 533 838. In contrast to RNAi, siRNA, siNA, antisense-nucleotides and ribozymes, aptamers do not degrade any target mRNA but interact specifically with the secondary and tertiary structure of a target compound such as a protein. Upon interaction with the target, the target typically shows a change in its biological activity. The length of aptamers typically ranges from as little as 15 to as much as 80 nucleotides, and preferably ranges from about 20 to about 50 nucleotides.

Another group of functional nucleic acids are spiegelmers as, for example, described in international patent application WO 98/08856. Spiegelmers are molecules similar to aptamers. However, spiegelmers consist either completely or mostly of L-nucleotides rather than D-nucleotides in contrast to aptamers. Otherwise, particularly with regard to possible lengths of spiegelmers, the same applies to spiegelmers as outlined in connection with aptamers.

As described herein, it has surprisingly been found that the compounds according to the present invention are particularly suitable to deliver nucleic acids, preferably functional nucleic acids such as siRNA and siNA molecules, into cells. As outlined in more detail in the examples below, the compounds according to the present invention are very active in delivering said nucleic acids into the intracellular space of cells.

It is to be acknowledged that the compounds according to the present invention are also beneficial insofar as they are particularly mild or non-toxic. Such lack of toxicity is clearly advantageous over the compounds of the prior art as they will significantly contribute to the medicinal benefit of any treatment using these kinds of compounds by avoiding side effects.

It is within the present invention that a composition and more particularly a pharmaceutical composition including any compounds of the invention may comprise one or more of the aforementioned biologically active compounds which may be contained in a composition according to the present invention as pharmaceutically active compound and as further constituent, respectively. It will be acknowledged by the ones skilled in the art that any of these compounds can, in principle, be used as a pharmaceutically active compound. Such pharmaceutically active compound is typically directed against a target molecule which is involved in the pathological mechanism of a disease. Due to the general design principle and mode of action underlying the various biologically active compounds and thus the pharmaceutically active compounds as used in connection with any aspect of the present invention, virtually any target can be addressed. Accordingly, the compounds according to the present invention and the respective compositions containing the same can be used for the treatment or prevention of any disease or diseased condition which can be addressed, prevented and/or treated using this kind of biologically active compounds. It is to be acknowledged that apart from these biologically active compounds also any other biologically active compound can be part of a composition according to any embodiment of the present invention. Preferably such other biologically active compound comprises at least one negative charge, preferably under conditions where such other biologically active compound is interacting or complexed with the compound according to the present invention, more preferably the compound according to the present invention which is present as a cationic lipid.

As used herein, a biologically active compound is preferably any compound which is biologically active, preferably exhibits any biological, chemical and/or physical effects on a biological system. Such biological system is preferably any biochemical reaction, any cell, preferably any animal cell, more preferably any vertebrate cell and most preferably any mammalian cell, including, but not limited to, any human cell, any tissue, any organ and any organism. Any such organism is preferably selected from the group comprising mice, rats, guinea pigs, rabbits, cats, dogs, monkeys and humans.

It is also within the present invention that any of the compositions according to the present invention, more particularly any pharmaceutical composition according to the present invention may comprise any further pharmaceutically active compound(s).

The pharmaceutical composition of the invention is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

The composition, particularly the pharmaceutical composition according to the present invention can be used for various forms of administration, whereby local administration and systemic administration are particularly preferred. Even more preferred is a route of administration which is selected from the group comprising intramuscular, percutaneous, subcutaneous, intravenous and pulmonary administration. As preferably used herein, local administration means that the respective composition is administered in close spatial relationship to the cell, tissue and organ, respectively, to which the composition and the biologically active compound, respectively, is to be administered. As used herein, systemic administration means an administration which is different from a local administration and more preferably is the administration into a body fluid such as blood and liquor, respectively, whereby the body liquid transports the composition to the cell, tissue and organ, respectively, to which the composition and the biologically active compound, respectively, is to be delivered.

Preferably, the composition of the invention—or the supramolecular complex according to the invention—is administered to a patient by pulmonary route. Accordingly, the composition of the invention as well as the supramolecular complex of the invention may be used in order to deliver an active molecule in a pulmonary cell of a patient in need thereof. The said composition may thus be used in a treatment of a pulmonary disorder such as lung cancer, asthma, chronic obstructive pulmonary disease (COPD), asthma, and lower respiratory infections and the like. In such a case, the composition of the invention may be formulated as a spray or an aerosol.

As used herein, the cell across the cell membrane of which a biologically active compound is to be transferred by means of the compound and composition according to the present invention, respectively, is preferably an eukaryotic cell, more preferably a vertebrate cell and even more preferably a mammalian cell. Most preferably the cell is a human cell.

Any medicament which can be manufactured using the compound and composition according to the present invention, respectively, is for the treatment and prevention of a patient. Preferably such patient is a vertebrate, more preferably a mammal and even more preferably such mammal is selected from the group comprising mice, rats, dogs, cats, guinea pigs, rabbits, monkeys and humans. In a further aspect the compound and composition according to the present invention can be used as a transferring agent, more preferably as a transfection agent.

As preferably used herein a transferring agent is any agent which is suitable to transfer a compound, more preferably a biologically active compound such as a pharmaceutically active compound across a membrane, preferably a cell membrane and more preferably transfer such compound into a cell as previously described herein. Preferably, the cells are endothelial cells, more preferably endothelial cells of vertebrates and most preferred endothelial cells of mammals such as mice, rats, guinea pigs, dogs, cats, monkeys and human beings.

In a still further aspect the present invention is related to a method for transferring, more particularly transfecting, a cell with a biologically active compound. In a first step, whereby the sequence of steps is not necessarily limited, the cell and the membrane and cell, respectively, is provided. In a second step, a compound according to the present invention is provided as well as a biologically active compound such as a pharmaceutically active compound. This reaction can be contacted with the cell and the membrane, respectively, and due to the biophysical characteristics of the compound and the composition according to the present invention, the biologically active compound will be transferred from one side of the membrane to the other one, or in case the membrane forms a cell, from outside the cell to within the cell. It is within the present invention that prior to contacting the cell and the membrane, respectively, the biologically active compound and the compound according to the present invention, i.e. the cationic lipid, are contacted, whereupon preferably a complex is formed and such complex is contacted with the cell and the membrane, respectively.

In a further aspect of the present invention the method for transferring a biologically active compound and a pharmaceutically active compound, respectively, comprises the steps of providing the cell and the membrane, respectively, providing a composition according to the present invention and contacting both the composition and the cell and the membrane, respectively. It is within the present invention that the composition may be formed prior or during the contacting with the cell and the membrane, respectively.

In an embodiment of any method for transferring a biologically active compound as disclosed herein, the method may comprise further steps, preferably the step of detecting whether the biologically active compound has been transferred. Such detection reaction strongly depends on the kind of biologically active compounds transferred according to the method and will be readily obvious for the ones skilled in the art. It is within the present invention that such method is performed on any cell, tissue, organ and organism as described herein.

A further aspect of the invention is a method preparing a pharmaceutical composition for delivering a pharmaceutically active compound to a cell, said method comprising mixing the compound of the invention with the pharmaceutically active compound.

In another aspect, the present invention also concerns a kit for preparing a composition for delivering a molecule of interest to a cell, said kit comprising at least one compound of the invention and a leaflet providing guidelines to use such a kit. Optionally, the kit may further comprise a buffer and/or a cell culture medium and/or solution isotonic to biological fluids. All embodiments disclosed above for the compound and the compositions of the invention are also encompassed in this aspect.

### IV. Method for Preparing the Compound According to the Invention

The hereunder examples provide several synthetic routes in order to obtain compounds of the invention, in particular compounds of formula (IV). Based on his/her general knowledge in organic chemistry, the one skilled in the art is able to adapt the said synthetic routes so as to obtain the desired compound of the invention.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

### A. Part 1

### Compounds of the Invention Wherein R⁸ is

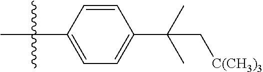 [SMILES: *c1ccc(C(C)(C)CC(C)(C)C)cc1]

### Materials and Methods

Unless otherwise stated, all chemical reagents were purchased from Alfa Aesar (Bischeim, France) and used without purification. When required, solvents were dried by standard procedures just before use [46]. 1,2-Dioleoyloxy-3-(N,N,N-trimethylamino)propyl chloride (DOTAP), 1,2-dioleoyl-sn-glycero-3-phosphoethanolamine (DOPE), 1,2-dioleoyl-sn-glycero-3-phosphocholine (DOPC), Triton X-100®, 3-(4,5-dimethylthiazol-2-yl)-2,5-diphenyl tetrazolium bromide (MTT) were from Sigma-Aldrich (Saint-Quentin Fallavier, France). Culture media Dulbecco's Modified Eagle Medium Glutamax (DMEM) and supplements were from GIBCO-BRL (Cergy-Pontoise, France). Fetal calf serum (FCS) was from Perbio (Brebières, France). Penicillin, streptomycin, L-glutamine, and sheep red blood cells (RBC) were from Eurobio (Courtaboeuf, France). Lysis and luciferin solutions for monitoring luciferase activity were purchased from Promega (Charbonnières, France). U87egfpluc cells were purchased from ATCC transformed as previously described [74]. The 16HBE14o- (16HBE) cells were a generous gift from Dr D. Gruenert (California Pacific Medical Center Research Institute, San Francisco, Calif., USA). The small double stranded siRNAs (e.g., PAGE-purified oligonucleotides terminated with two 2′-deoxythymidines at their 3′-ends, supplied at 100 μM and stored at −20° C.) were from Eurogentec (Angers, France).

Thin layer chromatography (TLC) was performed on precoated plates (0.25 mm Silica Gel 60, F₂₅₄, Merck, Darmstadt, Germany). Products were purified by flash chromatography over silica gel (Silica Gel 60, 40-63 μm, Merck, Darmstadt, Germany). NMR spectra were recorded on Bruker 300 MHz Avance DPX or Bruker 400 MHz Avance III instruments. ¹H—, ¹³C—, and ³¹P-NMR chemical shifts δ are reported in ppm relative to their standard reference (¹H: CHCl₃ at 7.27 ppm, HDO at 4.63 ppm, CD₂HOD at 3.31 ppm; ¹³C: CDCl₃ at 77.0 ppm, CD₃OD at 49.00 ppm; ³¹P: H₃PO₄ at 0.0 ppm). IR spectra were recorded on a FT-IR Nicolet 380 spectrometer in the ATR mode and absorptions values v are in wave numbers (cm⁻¹).

Mass Spectra (MS) were recorded on an Agilent Technologies 6520 Accurate Mass QToF, using electrospray ionization (ESI) mode. Mass data are reported in mass units (m/z). MALDI MS analyses were carried out on an Ultraflex™ MALDI-ToF/ToF instrument (Bruker Daltonics). The spectrometer was operated in positive reflectron mode with 25 kV applied to the target, and 26 kV applied to the reflectron. The laser was operated at 337 nm. The delayed extraction was optimized at 110 ns to obtain the best resolution on the reference compounds used for calibration and on the samples. The mass spectrometer was calibrated using a mixture containing 7 peptides (Bruker Peptide Calibration Standard #206196, Bruker Daltonics): bradykinin 1-7 (m/z=757.400), human angiotensin II (m/z=1046.542), human angiotensin I (m/z=1296.685), substance P (m/z=1347.735), bombesin (m/z=1619.822), renin (m/z=1758.933), ACTH 1-17 (m/z=2093.087), and ACTH 18-39 (m/z=2465.199). Dithranol (1,8,9-trihydroxyanthracene, 10 mg/ml) in dichloromethane was used as a matrix. The mass spectra were processed using the FlexAnalysis 2.4 build 11 software (Bruker Daltonics).

According to the details below, the following compounds were prepared:

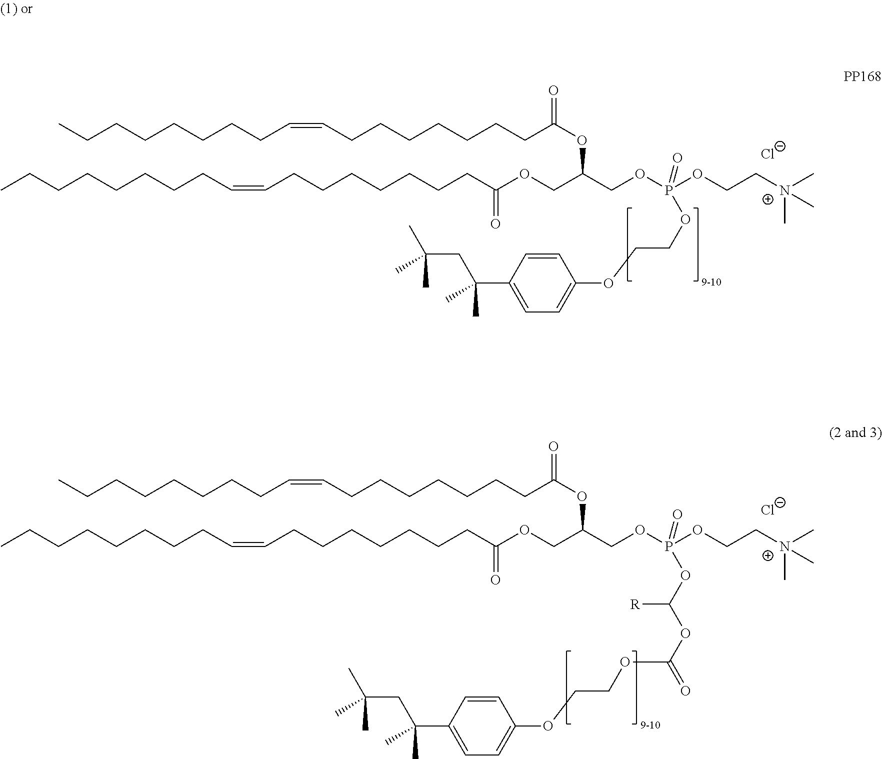

wherein with compound (2) or P111, R is hydrogen and with compound (3) or PP163, R is methyl,

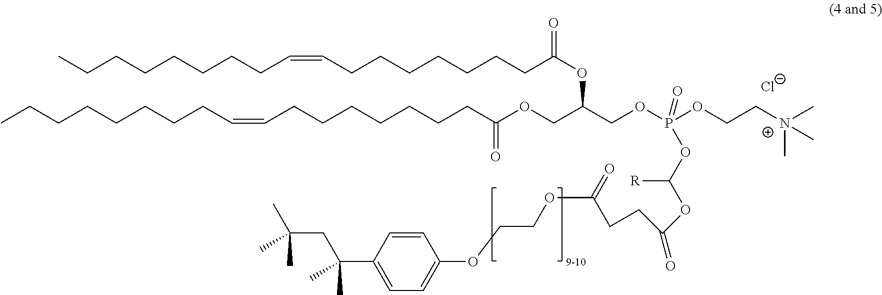

wherein with compound (4) or PP299, R is hydrogen and with compound (5) or PP303, R is methyl, and

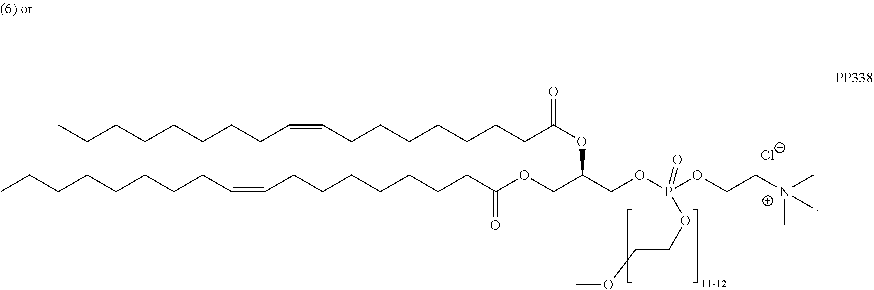

For reference, DOPC and EDOPC are as follows:

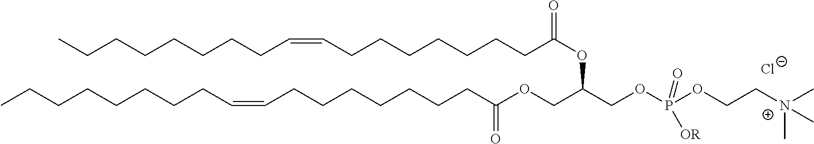

wherein with DOPC, R is hydrogen and with EDOPC, R is ethyl.

### 1.1 Synthesis and Characterization of Conjugate 1

Triflic anhydride (300 μL, 1.76 mmol) was added dropwise to a solution of Triton X-100® (1.00 g, 1.60 mmol) and pyridine (141 μL, 1.76 mmol) in freshly distilled CH₂Cl₂ (10.0 mL) at −50° C. The reaction mixture was stirred under argon at −50° C. for 2 h before it was poured into ice-cold water (10 mL). The organic layer was separated and washed with cold water (2×10 mL). The organic layer was dried over anhydrous magnesium sulfate, filtered and concentrated under reduced pressure. Triton X-100® trifluoromethanesulfonyl ester was obtained as a clear viscous oil (0.80 g, 64%) and was used in the next step without further purification. Due to relative instability of the compound, ¹H NMR spectrum was recorded immediately after isolation. ¹H-NMR (300 MHz, CDCl₃) δ 7.26 (d, ³J_{HH}=8.9 Hz, 2H), 6.81 (d, ³J_{HH}=8.9 Hz, 2H), 4.63 (t, ³J_{HH}=4.5 Hz, 2H), 4.11 (t, ³J_{HH}=4.5 Hz, 2H), 3.86-3.64 (m, 36H), 1.69 (s, 2H), 1.33 (s, 6H), 0.71 (s, 9H).

To a solution of DOPC (100 mg, 0.127 mmol) in freshly distilled CHCl₃ (3.0 mL) was added a solution of freshly prepared Triton X-100 trifluoromethanesulfonyl ester 7 (300 mg, 0.381 mmol) in dry CHCl₃ (2.0 mL). The resulting reaction mixture was stirred at room temperature for 20 h under argon. The reaction mixture was concentrated under reduced pressure at room temperature and the crude residue was purified by flash chromatography over silica gel (CH₂Cl₂/MeOH: 95/5 to 90/10) to yield compound 1 as a colorless oil (64 mg, 32%, 2 diastereomers) together with a fraction of unreacted DOPC (45 mg, 45%). TLC R_{f} 0.2 (CH₂Cl₂/MeOH 95:5). ¹H-NMR (400 MHz, CDCl₃/CD₃OD 1:1) δ 7.22 (d, ³J_{HH}=8.5 Hz, 2H), 6.78 (d, ³J_{HH}=8.5 Hz, 2H), 5.33-5.31 (m, 4H), 5.21 (s, 1H), 4.53 (br s, 2H), 4.31-4.06 (m, 8H), 3.82-3.80 (m, 4H), 3.68-3.59 (m, 24H), 3.28 (s, 9H), 2.30 (q, ³J_{HH}=7.6 Hz, 4H), 1.98 (q, ³J_{HH}=5.9 Hz, 8H), 1.67 (s, 2H), 1.57 (br s, 4H), 1.30-1.24 (m, 46H), 0.85 (t, ³J_{HH}=6.7 Hz, 6H), 0.68 (s, 9H). ¹³C-NMR (100 MHz, CDCl₃) δ 173.5, 173.1, 156.3, 143.0, 130.2, 129.8, 127.3, 114.0, 70.5, 70.2, 70.1, 70.0, 69.8, 69.7, 69.6, 69.5, 67.9, 67.6, 67.4, 66.2, 61.9, 57.1, 54.6, 38.1, 34.3, 34.1, 32.5, 32.1, 31.9, 29.9, 29.7, 29.5, 29.4, 29.3, 29.2, 27.4, 25.0, 22.9, 14.3. ³¹P-NMR (162 MHz, CDCl₃) δ −1.98, −1.99. ESI-MS (m/z): calc. for C₅₈H₁₀₅NO₈P(C₂H₄O)₈ 1326.97. found 1326.9 (75%, [M-TfO]⁺). calc. for C₅₈H₁₀₅NO₈P(C₂H₄O), 1282.94. found 1282.9 (82%, [M-TfO]⁺). calc. for C₅₈H₁₀₅NO₈P(C₂H₄O)₆ 1238.92. found 1238.9 (100%, [M-TfO]⁺). FT-IR (thin film) ν 2923, 2854, 1741, 1511, 1464, 1254, 1155, 1097, 1030, 638.

### 1.2 Synthesis and Characterization of Conjugate 2

To a solution of Triton X-100® (1.00 g, 1.60 mmol) and pyridine (161 μL, 2.00 mmol) in freshly distilled CH₂Cl₂ (10.0 mL) was added at once chloromethylchloroformate (148 μL, 1.60 mmol). The reaction mixture was stirred at room temperature for 2 h under a positive argon atmosphere, whereupon the mixture was quenched with water (20.0 mL) and extracted with EtOAc (2×20 mL). The organic layer was dried over Na₂SO₄, filtered, and volatiles were removed under reduced pressure. Triton X-100 chloromethyl carbonate 8 was recovered as a clear viscous oil (1.19 g, 99%) and was used in the next step without further purification. ¹H-NMR (400 MHz, CDCl₃) δ 7.22 (d, ³J_{HH}=8.9 Hz, 2H), 6.79 (d, ³J_{HH}=8.9 Hz, 2H), 5.71 (s, 2H), 4.33 (t, ³J_{HH}=4.5 Hz, 2H), 4.08 (t, ³J_{HH}=4.3 Hz, 2H), 3.83 (t, ³J_{HH}=4.5 Hz, 2H), 3.73-3.62 (m, 32H), 1.67 (s, 2H), 1.31 (s, 6H), 0.68 (s, 9H). ¹³C-NMR (100 MHz, CDCl₃) δ 156.6, 153.6, 142.6, 127.2, 113.9, 72.4, 71.0, 70.9, 70.8, 70.0, 68.8, 68.3, 67.5, 57.2, 38.1, 32.5, 31.9, 31.8.

To a solution of DOPC (146 mg, 0.186 mmol) in freshly distilled CHCl₃ (1.50 mL) was added a solution of compound 8 (1.10 g, 1.49 mmol) in dry CHCl₃ (1.50 mL). The resulting reaction mixture was heated at reflux for 20 h under a positive argon atmosphere, then cooled down and concentrated under reduced pressure at 20-25° C. The residue was purified over silica gel by flash chromatography (CH₂Cl₂/MeOH 88:12 to 80:20) to yield compound 2 as a slightly yellow oil (65 mg, 23%, 2 diastereomers) together with a fraction of unreacted DOPC (80 mg, 55%). TLC R_{f} 0.2 (CH₂Cl₂/MeOH 88:12). ¹H-NMR (400 MHz, CDCl₃/CD₃OD 1:1) δ 7.23 (d, ³J_{HH}=8.3 Hz, 2H), 6.78 (d, ³J_{HH}=8.4 Hz, 2H), 5.68-5.63 (m, 2H), 5.31-5.29 (m, 5H), 4.53 (s, 2H), 4.34-4.08 (m, 8H), 3.81-3.66 (m, 36H), 3.24 (s, 9H), 2.34-2.27 (m, 4H), 1.98-1.96 (m, 8H), 1.67 (s, 2H), 1.60-1.55 (m, 4H), 1.29-1.22 (m, 46H), 0.84 (t, ³J_{HH}=6.5 Hz, 6H), 0.66 (s, 9H). ¹³C-NMR (100 MHz, CDCl₃/CD₃OD 1:1) δ 174.1, 173.7, 156.7, 153.9, 143.1, 130.5, 130.1, 127.6, 114.3, 86.6, 71.0, 70.8, 70.7, 70.3, 69.8, 69.0, 68.5, 67.8, 67.1, 66.0, 65.9, 62.4, 62.0, 57.4, 54.6, 38.3, 34.5, 34.4, 32.7, 32.3, 32.1, 32.0, 30.2, 30.1, 29.9, 29.7, 29.6, 29.5, 27.7, 27.6, 25.3, 23.1, 14.3. ³¹P-NMR (162 MHz, CDCl₃/CD₃OD 1:1) δ −3.44, −3.53. ESI-MS (m/z): calc. For C₆₀H₁₀₇NO₁₁P(C₂H₄O)₁₀1489.02. found 1489.0 (64%, [M-Cl]⁺). calc. for C₆₀H₁₀₇NO₁₁P(C₂H₄O)₉ 1444.99. found 1444.9 (87%, [M-Cl]⁺). calc. for C₆₀H₁₀₇NO₁₁P(C₂H₄O)₈ 1400.97. found 1400.9 (100%, [M-Cl]⁺). FT-IR (thin film) ν 2924, 2854, 1743, 1511, 1465, 1364, 1248, 1109, 1044, 991.

### 1.3 Synthesis and Characterization of Conjugate 3

To a solution of Triton X-100® (5.00 g, 8.01 mmol) and pyridine (807 μL, 10.0 mmol) in freshly distilled CH₂Cl₂ (50.0 mL) was added at once chloroethylchloroformate (864 μL, 8.01 mmol). The reaction mixture was stirred at room temperature for 2 h under a positive argon atmosphere, whereupon the mixture was quenched with water (100 mL), and extracted with EtOAc (2×100 mL). The organic layer was dried over Na₂SO₄, filtered and volatiles were removed under reduced pressure. Expected Triton X-100 1-chloroethyl carbonate 9 was obtained as a clear viscous oil (5.85 g, 97%) and was used in the next step without further purification. ¹H-NMR (400 MHz, CDCl₃) δ 7.23 (d, ³J_{HH}=8.9 Hz, 2H), 6.79 (d, ³J_{HH}=8.9 Hz, 2H), 6.39 (q, ³J_{HH}=6.0 Hz, 1H), 4.32 (t, ³J_{HH}=4.7 Hz, 2H), 4.08 (t, ³J_{HH}=4.5 Hz, 2H), 3.81 (t, ³J_{HH}=4.5 Hz, 2H), 3.73-3.61 (m, 34H), 1.80 (d, ³J_{HH}=5.8 Hz, 3H), 1.66 (s, 2H), 1.31 (s, 6H), 0.67 (s, 9H). ¹³C-NMR (100 MHz, CDCl₃) δ 156.6, 153.1, 142.5, 127.2, 113.9, 84.8, 71.0, 70.9, 70.8, 70.7, 70.0, 68.8, 68.0, 67.4, 57.2, 38.1, 32.5, 31.9, 31.8, 25.4.

To a solution of DOPC (400 mg, 0.51 mmol) in freshly distilled CHCl₃ (5.0 mL) was added a solution of compound 9 (3.07 g, 4.07 mmol) in dry CHCl₃ (3.0 mL). The resulting reaction mixture was heated at reflux for 20 h under a positive argon atmosphere, cooled down, and concentrated under reduced pressure at 20-25° C. The residue was purified by silica gel chromatography (CH₂Cl₂/MeOH 88:12 to 80:20) to yield pure CPDC 3 as a colorless oil (280 mg, 35%, 4 diastereomers) together with a fraction of unreacted DOPC (207 mg, 52%). TLC R_{f} 0.2 (CH₂Cl₂/MeOH 88:12). ¹H-NMR (400 MHz, CDCl₃) δ 7.22 (d, ³J_{HH}=8.9 Hz, 2H), 6.78 (d, ³J_{HH}=8.9 Hz, 2H), 6.29 (br s, 1H), 5.33-5.21 (m, 5H), 4.55 (br s, 2H), 4.30-4.24 (m, 6H), 4.07 (t, ³J_{HH}=4.9 Hz, 2H), 3.81 (t, ³J_{HH}=4.5 Hz, 2H), 3.69-3.61 (m, 36H), 3.47 (s, 9H), 2.31-2.28 (m, 4H), 1.98 (m, 8H), 1.66 (s, 2H), 1.59 (br s, 7H), 1.30-1.24 (m, 46H), 0.85 (t, ³J_{HH}=6.8 Hz, 6H), 0.67 (s, 9H). ¹³C-NMR (100 MHz, CDCl₃) δ 173.5, 173.1, 156.6, 153.3, 153.1, 142.6, 130.2, 129.8, 127.2, 113.9, 95.7, 95.4, 70.9, 70.7, 70.0, 68.8, 67.5, 65.5, 65.4, 61.7, 61.6, 57.2, 54.7, 38.1, 34.3, 34.2, 32.5, 32.1, 31.9, 31.8, 29.9, 29.7, 29.5, 29.4, 29.3, 29.2, 27.4, 27.3, 25.0, 22.8, 21.4, 14.3. ³¹P-NMR (162 MHz, CDCl₃) δ −5.36, −5.50, −5.80, −6.00. ESI-MS (m/z): calc. for C₆₁H₁₀₉NO₁₁P(C₂H₄O)₁₀ 1503.06. found 1503.0 (65%, [M-Cl]⁺). calc. for C₆₁H₁₀₉NO₁₁P(C₂H₄O)₉ 1459.01. found 1459.0 (90%, [M-Cl]⁺). calc. for C₆₁H₁₀₉NO₁₁P(C₂H₄O)₈ 1414.98. found 1414.9 (100%, [M-Cl]⁺). FT-IR (thin film) ν 2924, 2855, 1743, 1512, 1465, 1258, 1108, 977.

### 1.4 Synthesis and Characterization of Conjugate 4

A mixture of Triton X-100® (5.00 g, 8.01 mmol), DMAP (1.00 g, 8.0 mmol) and succinic anhydride (2.00 g, 20.0 mmol) in dry toluene (100 mL) was refluxed under an argon atmosphere for 18 h. The reaction mixture was cooled down and concentrated under reduced pressure. The crude residue was purified over silica gel (CH₂Cl₂/MeOH 90:10) to yield intermediate Triton X-100 hemisuccinate as a colorless oil (6.00 g, 99%). TLC R_{f} 0.57 (CH₂Cl₂/MeOH 90:10). ¹H-NMR (400 MHz, CDCl₃) δ 7.22 (d, ³J_{HH}=8.9 Hz, 2H), 6.79 (d, ³J_{HH}=8.8 Hz, 2H), 4.23 (t, ³J_{HH}=4.6 Hz, 2H), 4.08 (t, ³J_{HH}=5.1 Hz, 2H), 3.82 (t, ³J_{HH}=4.9 Hz, 2H), 3.70-3.61 (m, 32H), 2.62 (m, 4H), 1.66 (s, 2H), 1.31 (s, 6H), 0.67 (s, 9H). ¹³C-NMR (100 MHz, CDCl₃) δ 172.2, 156.6, 142.6, 127.2, 113.9, 70.9, 70.8, 70.7, 70.0, 69.2, 67.5, 64.0, 57.2, 38.1, 32.5, 31.9, 31.8, 29.6, 29.2, 28.9.

To a suspension of the previous ester (1.00 g, 1.34 mmol) in water (10 mL) was added n-Bu₄NHSO₄ (88 mg, 0.268 mmol) and Na₂CO₃ (568 mg, 5.36 mmol), and the reaction mixture was stirred for 20 min at room temperature affording a clear solution. Then, the mixture was cooled down to 0° C. and a solution of chloromethylchlorosulfate (160 μL, 1.74 mmol) in dry CH₂Cl₂ (20 mL) was added dropwise over 5 min. The resulting white suspension was vigorously stirred for 1 h at 0° C., and for 18 h at room temperature. The organic layer was decanted, and the aqueous phase was extracted with CH₂Cl₂ (2×20 mL). Combined organic layers were dried over Na₂SO₄, filtered and concentrated under reduced pressure. The crude residue was purified by flash chromatography over silica gel (CH₂Cl₂/MeOH 95:5) to yield Triton X-100 chloromethyl succinate 10 as a colorless oil (968 mg, 91%). TLC R_{f} 0.5 (CH₂Cl₂/MeOH 95:5). ¹H-NMR (400 MHz, CDCl₃) δ 7.23 (d, ³J_{HH}=8.9 Hz, 2H), 6.79 (d, ³J_{HH}=8.9 Hz, 2H), 5.69 (s, 2H), 4.23 (t, 3J_{HH}=4.9 Hz, 2H), 4.08 (t, ³J_{HH}=4.9 Hz, 2H), 3.82 (t, ³J_{HH}=4.8 Hz, 2H), 3.70-3.61 (m, 32H), 2.68 (s, 4H), 1.67 (s, 2H), 1.31 (s, 6H), 0.68 (s, 9H). ¹³C-NMR (100 MHz, CDCl₃): δ 173.1, 170.6, 156.6, 142.1, 127.2, 113.9, 71.0, 70.8, 70.0, 69.2, 68.9, 67.5, 64.2, 57.2, 38.1, 32.5, 31.9, 31.8, 29.2, 28.8.

To a solution of DOPC (400 mg, 0.51 mmol) in freshly distilled CHCl₃ (2.0 mL) was added a solution of compound 10 (860 mg, 1.08 mmol) in dry CHCl₃ (1.0 mL). The resulting reaction mixture was heated at reflux for 18 h under a positive argon atmosphere. Then, it was cooled down, concentrated under reduced pressure at 20-25° C. and the crude residue was purified by flash chromatography over silica gel (CH₂Cl₂/MeOH 88:12) to 80:20). Compound 4 was obtained as a colorless oil (188 mg, 24%, 2 diastereomers) together with a fraction of unreacted DOPC (191 mg, 48%). TLC R_{f} 0.25 (CH₂Cl₂/MeOH 85:15). ¹H-NMR (400 MHz, CDCl₃/CD₃OD 1:1) δ 7.23 (d, ³J_{HH}=8.9 Hz, 2H), 6.79 (d, ³J_{HH}=8.9 Hz, 2H), 5.65 (m, 2H), 5.29-5.31 (m, 5H), 4.52 (br s, 2H), 4.20-4.52 (m, 8H), 4.09 (t, ³J_{HH}=4.7 Hz, 2H), 3.82 (t, ³J_{HH}=4.4 Hz, 2H), 3.61-3.71 (m, 36H), 3.24 (s, 9H), 2.69 (s, 4H), 2.28-2.33 (m, 4H), 1.95-1.98 (m, 8H), 1.67 (s, 2H), 1.58 (m, 4H), 1.24-1.30 (m, 46H), 0.84 (t, ³J_{HH}=7.1 Hz, 6H), 0.67 (s, 9H). ¹³C-NMR (100 MHz, CDCl₃) δ 174.2, 173.8, 173.0, 171.7, 156.8, 143.1, 130.5, 130.1, 127.6, 114.3, 83.7, 83.6, 71.1, 71.0, 70.9, 70.8, 70.7, 70.6, 70.3, 70.0, 69.9, 69.8, 69.5, 69.4, 67.8, 67.1, 66.1, 64.6, 62.4, 62.2, 57.5, 54.6, 38.4, 34.6, 34.5, 32.7, 32.4, 32.1, 323.0, 30.2, 29.9, 29.8, 29.7, 29.6, 29.5, 29.1, 28.8, 27.7, 27.6, 25.3, 23.1, 14.4. ³¹P-NMR (162 MHz, CDCl₃) δ −3.21, −3.15. ESI-MS (m/z): calc. for C₆₃H₁₁₁NO₁₂P(C₂H₄O)₉ 1501.02. found 1501.0 (81%, [M-Cl]⁺). calc. for C₆₃H₁₁₁NO₁₂P(C₂H₄O)₈ 1456.99. found 1456.9 (98%, [M-Cl]⁺). calc. for C₆₃H₁₁₁NO₁₂P(C₂H₄O)₇ 1412.97. found 1412.9 (100%, [M-Cl]⁺). FT-IR (thin film) ν 2924, 2855, 1733, 1716, 1684, 1508, 1457, 1244, 1108, 1015, 668.

### 1.5 Synthesis and Characterization of Conjugate 5

A solution of Triton X-100 hemisuccinate (vide supra; 1.00 g, 1.34 mmol) in anhydrous dichloromethane (5 mL) was treated with oxalyl chloride (150 μL, 1.61 mmol) at 0° C. under an argon atmosphere. After 30 minutes, the reaction mixture was allowed to warm to room temperature and was stirred for 1 h. Volatiles were removed under reduced pressure affording the crude succinyl chloride intermediate that was used without further purification (1.03 g, 100%). ¹H-NMR (400 MHz, CDCl₃) δ 7.22 (d, ³J_{HH}=8.9 Hz, 2H), 6.79 (d, ³J_{HH}=8.8 Hz, 2H), 4.24 (dd, ³J_{HH}=3.9 and 5.2 Hz, 2H), 4.08 (t, ³J_{HH}=4.6 Hz, 2H), 3.82 (dd, ³J_{HH}=3.9 and 5.4 Hz, 2H), 3.69-3.61 (m, 32H), 3.19 (t, ³J_{HH}=6.6 Hz, 2H), 2.69 (t, ³J_{HH}=6.7 Hz, 2H), 1.66 (s, 2H), 1.31 (s, 6H), 0.7 (s, 9H). ¹³C-NMR (100 MHz, CDCl₃) δ 173.1, 171.0, 156.6, 142.6, 127.2, 113.9, 71.0, 70.8, 70.0, 69.1, 67.5, 64.4, 57.2, 41.9, 38.1, 32.5, 31.9, 31.8, 29.5.

Acetaldehyde (0.1 mL, 1.76 mmol) was added dropwise at 0° C. under inert argon atmosphere to a mixture of ZnCl₂ (3.0 mg, 22 μmol) and the freshly prepared succinyl chloride derivative (1.00 g, 1.34 mmol). After 1 h at 0° C., the reaction mixture was stirred at room temperature for 18 h. The crude mixture was directly purified by chromatography over silica gel (CH₂Cl₂/MeOH 95:5) to yield Triton X-100 1-chloroethyl succinate 11 as a colorless oil (795 mg, 72%). TLC R_{f} 0.55 (CH₂Cl₂/MeOH 95:5). ¹H-NMR (400 MHz, CDCl₃) δ 7.23 (d, ³J_{HH}=9.0 Hz, 2H), 6.79 (d, ³J_{HH}=8.9 Hz, 2H), 6.52 (q, ³J_{HH}=5.8 Hz, 1H), 4.23 (t, ³J_{HH}=4.8 Hz, 2H), 4.08 (t, ³J_{HH}=4.9 Hz, 2H), 3.82 (t, ³J_{HH}=5.2 Hz, 2H), 3.70-3.61 (m, 32H), 2.66-2.65 (m, 4H), 1.77 (d, ³J_{HH}=5.8 Hz, 3H), 1.67 (s, 2H), 1.31 (s, 6H), 0.68 (s, 9H). ¹³C-NMR (100 MHz, CDCl₃) δ 172.1, 170.2, 156.6, 142.6, 127.2, 114.0, 81.1, 71.0, 70.8, 70.1, 69.2, 67.5, 64.2, 57.2, 38.1, 32.5, 29.3, 28.9, 25.4.

To a solution of DOPC (437 mg, 0.556 mmol) in freshly distilled CHCl₃ (10.0 mL) was added a solution of compound 11 (3.60 g, 4.45 mmol) in dry CHCl₃ (4.0 mL). The resulting reaction mixture was heated at reflux for 3 h under an argon atmosphere, cooled down, and concentrated under reduced pressure at 20-25° C. The crude residue was purified by flash chromatography over silica gel (CH₂Cl₂/MeOH 88:12 to 80:20) to yield CPDC 5 as colorless oil (330 mg, 37%, 4 diastereomers) together with a fraction of unreacted DOPC (214 mg, 49%). TLC R_{f} 0.25 (CH₂Cl₂/MeOH 85:15). ¹H-NMR (400 MHz, CDCl₃) δ 7.22 (d, ³J_{HH}=8.9 Hz, 2H), 6.80 (d, ³J_{HH}=8.8 Hz, 2H), 6.44 (m, 1H), 5.30-5.32 (m, 5H), 4.52 (m, 2H), 4.20-4.30 (m, 8H), 4.07 (t, ³J_{HH}=4.7 Hz, 2H), 3.81 (t, ³J_{HH}=4.7 Hz, 2H), 3.61-3.69 (m, 38H), 3.46 (s, 9H), 2.64 (s, 4H), 2.29 (m, 4H), 1.97 (m, 8H), 1.67 (s, 2H), 1.55 (m, 7H), 1.24-1.30 (m, 46H), 0.85 (t, ³J_{HH}=6.6 Hz, 6H), 0.67 (s, 9H). ¹³C-NMR (100 MHz, CDCl₃) δ 173.5, 173.1, 172.4, 171.1, 156.6, 142.6, 130.2, 129.8, 127.2, 114.0, 92.0, 91.9, 71.0, 70.8, 70.7, 70.0, 69.4, 69.2, 69.1, 67.5, 66.7, 65.6, 64.4, 64.3, 61.8, 57.2, 54.7, 38.1, 34.4, 34.2, 32.5, 32.1, 31.9, 31.8, 29.9, 29.7, 29.5, 29.4, 29.3, 29.2, 29.1, 29.0, 28.7, 28.6, 27.4, 27.3, 25.0, 22.8, 21.4, 14.3. ³¹P-NMR (162 MHz, CDCl₃) δ −5.46, −5.63, −5.94, −6.15. ESI-MS (m/z): calc. for C₆₃H₁₁₃NO₁₂P(C₂H₄O)₁₀ 1559.06. found 1559.0 (98%, [M-Cl]⁺). calc. for C₆₃H₁₁₃NO₁₂P(C₂H₄O)₉ 1515.04. found 1515.0 (100%, [M-Cl]⁺). FT-IR (thin film) ν 2923, 2854, 1736, 1652, 1509, 1457, 1246, 1144, 1106, 1054, 977.

### 1.6 Synthesis and Characterization of Conjugate 6

To a solution of polyethylene glycol monomethylether 550 (1.00 g, 1.81 mmol) and pyridine (160 μL, 2.00 mmol) in freshly distilled CH₂Cl₂ (10.0 mL) was added dropwise triflic anhydride (340 μL, 2.00 mmol) at −50° C. The reaction mixture was stirred at −50° C. for 2 h under argon, and then poured into ice-cold water (10 mL). The organic layer was separated and extracted with cold water (2×10 mL). The organic layer was dried over Na₂SO₄, filtered and concentrated under reduced pressure. The expected sulfonyl ester was obtained as a clear viscous oil (1.10 g, 86%) and was used in the next step without further purification. Due to relative instability of this compound, ¹H NMR spectrum was recorded immediately after isolation. ¹H-NMR (300 MHz, CDCl₃) δ 4.59 (t, ³J_{HH}=4.9 Hz, 2H), 3.79 (t, ³J_{HH}=5.0 Hz, 2H), 3.49-3.66 (m, 48H), 3.33 (s, 3H). The previous compound (1.10 g, 1.57 mmol) in dry CHCl₃ (5.0 mL) was added to a solution of DOPC (300 mg, 0.40 mmol) in freshly distilled CHCl₃ (10.0 mL). The resulting reaction mixture was stirred at room temperature for 20 h under argon. Then, the reaction mixture was concentrated under reduced pressure at room temperature and the residue was purified by silical gel chromatography (CH₂Cl₂/MeOH 95:5 to 80:20). Compound 6 was obtained as a colorless oil (291 mg, 49%, 2 diastereomers) together with a fraction of unreacted DOPC (114 mg, 38%). TLC R_{f} 0.31 (CH₂Cl₂/MeOH 90:10). ¹H-NMR (400 MHz, CDCl₃) δ 5.29-5.32 (m, 4H), 5.19-5.23 (m, 1H), 4.52 (m, 2H), 4.12-4.31 (m, 6H), 3.83 (br s, 2H), 3.57-3.69 (m, 52), 3.50-3.52 (m, 2H), 3.33 (s, 3H), 3.27 (s, 9H), 2.27 (m, 4H), 1.96 (m, 8H), 1.57 (m, 4H), 1.23-1.26 (m, 40H), 0.85 (t, ³J_{HH}=6.7 Hz, 3H). ¹³C-NMR (100 MHz, CDCl₃) δ 173.4, 173.1, 130.2, 129.9, 72.6, 72.1, 72.0, 70.3, 69.9, 69.6, 69.5, 67.9, 67.8, 66.2, 65.7, 61.9, 61.8, 61.7, 59.2, 54.6, 34.3, 34.2, 32.1, 29.9, 29.7, 29.5, 29.4, 29.3, 29.2, 27.4, 27.3, 25.0, 22.8, 14.3. ³¹P-NMR (162 MHz, CDCl₃) δ −1.91, −1.94. ESI-MS (m/z): calc. for C₄₅H₈₇NO₈P(C₂H₄O)₁₂ 1328.93. found 1328.9 (68%, [M-TfO]⁺). calc. for C₄₅H₈₇NO₈P(C₂H₄O)₁₁ 1284.9. found 1284.9 (84%, [M-TfO]⁺). calc. for C₄₅H₈₇NO₈P(C₂H₄O)₁₀ 1240.88. found 1240.9 (94%, [M-TfO]⁺). calc. for C₄₅H₈₇NO₈P(C₂H₄O)₉ 1196.85. found 1196.9 (100%, [M-TfO]⁺). FT-IR (thin film) ν 2924, 2855, 1743, 1261, 1106, 1031, 722, 668, 639.

### 2. Liposome and Lipoplex Preparation

For hydrolysis rate measurements using ³¹P-NMR, liposomes were obtained by a solvent injection technique. [47] The lipids (10 μmol) were dissolved in PrOH (200 μL) and then injected with a syringe with a flow rate of ca. 600 μL/min and a stirring speed of 400 rpm into the appropriate aqueous buffer medium (either Hepes 10 mM pH 7.4 or AcOK/AcOH 10 mM pH 4.5). For biological assays with lipids alone, lipids were suspended in aqueous phase by mixing the desired amount of lipid (4 mM in EtOH) with 40 μL of 4.5% glucose and incubated for 15 min at room temperature before use. Lipoplexes were prepared using the same procedure except that the 4.5% glucose contained 120 nM siRNA.

3. siRNA Transfection

The luciferase gene silencing experiments were performed with a RNA duplex (siLuc) of the sense sequence: 5′-CUU ACG CUG AGU ACU UCG A. Untargeted RNA duplex (sic) was of sequence: 5′-CGU ACG CGG AAU ACU UCG A. Cells were maintained at 37° C. in a 5% CO₂ humidified atmosphere and grown in DMEM medium with 10% FBS, 100 units/mL penicillin, 100 μg/mL streptomycin and 2 mM L-glutamine. U87 cells (human glioblastoma ATCC HTB-14) were transformed to stably express the Photinus pyralis luciferase-enhanced green fluorescence protein fusion gene originating from the pEGFPluc plasmid (Clontech, Mountain View, Calif.). The plasmid coded as well for a resistance gene to G418, selection was thus performed by adding 0.8 mg/mL G418 to the cell culture medium. The day before experiments, U87egfpluc cells were seeded into 96-well plates at a density of 8,000 cells per well in 100 μL cell culture medium. Lipids were freshly solubilized in ethanol at 2 mM concentration. Typically, 40 μL of 100 nM solution of siRNA (either siLuc or sic) in 4.5% glucose was added to the lipid (either 2, 4 or 8 μL deposited at the bottom of a 200 μL eppendorf tube). After agitation, the complexes (11 μL containing 16.17 ng siRNA) were added into each well (triplicate) by dilution with the cells. Cells were then let to grow in the incubator without further handling. Luciferase gene expression was assessed 48 h later using a commercial kit according to the manufacturer's protocol (Promega, Charbonnières, France). Briefly, culture medium was removed, and cells were washed with PBS (100 μL) and lyzed with 20 μL of the Promega lysis buffer. After agitation for 15 min, 200 μL of PBS were added. A volume of 5 μL was transferred to a white microplate and luminescence was read during 0.1 s with a luminometer (Berthold Centro LB960 XS, Thoiry, France) using 40 μL of the luciferin substrate. The transfection efficiency was expressed as the residual luciferase activity when compared to non-treated cells (100%). Value of each sample is the means of triplicate determinations (±SD).

### 4.1 Cell Cytotoxicity Assays

Mitochondrial activity measurements (MTT assay) and LDH release from 16HBE cells were used to assess cytotoxicity of the formulations.

### 4.2 Cell Mitochondrial Activity Measurements

Cells were grown in culture flasks (Becton-Dickinson) with DMEM culture medium supplemented with 10% FBS, 100 U/mL penicillin, 100 mg/mL streptomycin, 2 mM L-glutamine, and 5 mM Hepes. Culture was carried out at 37° C., in a 5% CO₂ and humidified atmosphere. At confluence, cells were released with trypsin (0.5% in PBS), counted and transferred into a 96-well plate (Becton-Dickinson) at a density of 6,000 cells/well. On the next day (24 h later), culture medium was changed to fresh DMEM medium supplemented with 10% FBS before addition of lipoplexes (100 μL) prepared as described above. After a 48 h incubation period at 37° C., culture supernatant was removed, cells were carefully washed with PBS and 0.5 mg/mL MTT (1 mL) in complete culture medium was added. After a 2 h incubation period at 37° C., MTT solution was removed and DMSO (1 mL) was added to lyse cells and dissolve reduced MTT. Intensity of MTT reduction was then evaluated by measuring absorbance at 570 nm. Viability of cells treated with lipoplexes was expressed as the percentage of the absorbance measured in untreated cells. Value for each sample is the mean of triplicate determinations (±SD).

### 4.3 Lactate Dehydrogenase (LDH) Assay

16HBE cells were seeded in culture plates and put in contact with lipoplexes as described above. After incubation of the cells with lipoplexes for the indicated period of time, aliquots of cell supernatants were transferred into a microliter plate, and LDH activity was measured using a commercial kit (Cytotoxicity Detection Kit Plus, Roche Applied Science, Meylan, France) according to the manufacturer's instructions. LDH activity was expressed as the percentage of the maximal LDH release obtained by complete cell lysis obtained with the kit lysis solution. Value for each sample is the mean of triplicate determinations (±SD).

### 5. Erythryocyte Leakage Assay

Hemolysis experiments were carried out according to a procedure described elsewhere. [48] For recovery from washing steps, sheep red blood cells were centrifuged at 400 RCF for 10 min, and washed three times with 150 mM NaCl. Cells were then resuspended in 100 mM PBS (pH 7.4), prepared to be isoosmotic to the inside of RBC and caused negligible hemolysis, and plated in 96-well plates to obtain 15×10⁶ cells in 50 μL. Increasing amounts of liposomes or lipoplexes (prepared at N/P 25) in PBS were added to the erythrocytes and incubated for 2 h at 37° C. and 5% CO₂. The release of hemoglobin was determined after centrifugation at 700 RCF for 10 min, by spectrophotometric analysis of the supernatant at 550 nm (Bio-Rad model 550 spectrophotometer, Marnes-la-Coquette, France). Complete hemolysis was achieved using deionized water yielding the 100% control value. The negative control was obtained by suspension of RBC in phosphate buffer alone. The experiments were performed in triplicate.

### 6. Lipid Metabolization and MALDI-ToF MS Analysis

The night before experiment, U87egfpluc cells were seeded into 12-well plates at a density of 100,000 cells per well in 1 mL of cell culture medium. To evaluate the degradation profile in the cell culture medium alone, a 12-well plate was prepared to contain cell culture medium without cells (1 mL). The lipid (70 μL, 4 mM) in ethanol was introduced in a 1.5 mL eppendorf tube. After solvent evaporation, siLuc (700 μL, 100 nM) in 4.5% glucose was added and vortexed. The resulting lipoplexes (100 μL) were added into wells by dilution with the cell culture medium containing 10% FBS (1 mL). After a 5 h incubation period in the cell incubator to allow lipoplexes to settle down, the medium (1 mL) was recovered into a glass vial. The cells were then washed twice with PBS (1 mL) to remove unbound lipoplexes and further incubated for 0, 5, 19 and 43 h in cell culture medium. For extraction of the lipids, the cell culture medium was carefully removed and the cells were recovered from the plate in 1 mL PBS by scrapping with a rubber policeman. The cell suspension was then transferred to a glass vial. Addition of CHCl₃/MeOH (2/1 v/v, 2 mL), followed by vigorous agitation and phase separation by centrifugation (500 RCF, 5 min) yielded a clear organic layer that was transferred into a glass vial and immediately frozen on dry ice and stored at −20° C. until mass spectrometry analysis. The sample (1 μl) was loaded on the target and, after evaporation of the solvent, the matrix (1 μl) was deposited on the residue.

### Example 1

### Design of the Cationic Lipid-Detergent Conjugates

Diacylglycerophosphocholines (PCs) are normal cellular metabolites and major constituents of membranes. Their zwitterionic polar head offers the opportunity to generate cationic lipids by esterification of the phosphate group. Gorman et al. first demonstrated that a PC-derived phosphotriester (1,2-dimyristoyl-sn-glycero-3-ethylphosphocholine, EDMPC) can mediate efficient gene transfer.[35] Later on, derivatives of DOPC and other PCs have been developed by McDonald et al. and their physical and transfection properties extensively investigated.[36-45] It was hypothesized that the covalent hybridization of a detergent molecule and a natural phospholipid to form a cationic phospholipid-detergent conjugate (CPDC) should improve endosomal escape of lipoplexes prepared thereof, and consequently might improve transfection efficiency.

This hypothesis relies on the cleavage of a linker inserted in between the cationic lipid and the detergent species. Considering the potential toxicity of non-natural cationic lipids, we designed the compounds so they produce innocuous DOPC upon cleavage, together with Triton X-100® (TX100). On the one hand, transformation of the cationic conjugate into zwitterionic DOPC prevents further interaction with the nucleic acid payload. On the other hand, released TX100 may have improved beneficial effect on membrane permeation and siRNA cytosol delivery since TX100 does not solubilize cholesterol-enriched raft domains (London, E., D. A. Brown, Biochim. Biophys. Acta 2000, 1508, 182) that might be involved in cationic lipoplexes internalization (Dalkara, D., G. Zuber, J. P. Behr, Mol. Ther. 2004, 9, 964). In order to control or modulate the susceptibility of the linker towards cleavage, TX100-DOPC conjugates were designed that present various structural features. The conjugate 1 is a “simple” TX100 phosphotriester that might be hypothetically cleaved by intracellular enzymes with release of free TX100.

Conjugates 2-5 incorporate an acetal moiety that is basically an acid-labile functional group. The rationale for these four compounds is that after cell internalization by endocytosis, lipoplexes entrapped in endocytic vesicle are delivered to “early” (or “sorting”) endosomes that are acidified by ATP-dependent proton pumps (pH˜6). The early endosomes then mature into late endosomes (pH˜5) that, after fusion with prelysosomal vesicles containing acid hydrolases, generate a harsh environment prone to degradation of the internalized cargo.[49, 50] Consequently, all along the endosome maturation pathway, lipoplexes are exposed to increasing acidity and conjugates 2-5 are expected to hydrolyze at different rates with respect to the nature of the functional group connecting TX100 to the acetal moiety (carbonate: compounds 2 and 3, or ester: compounds 4 and 5) and to the substitution on the acetal carbon atom (H: compounds 2 and 4, or Me: compounds 3 and 5).[51, 52] Besides, phosphoryloxymethyl esters are known to be substrates for intracellular esterases,[53-58] as it is the case for phosphoryloxy carbonates though it is much less documented.[59, 60] That may present an opportunity for amplification of TX100 release in case the endosomal compartment retains integrity up to fusion with the esterase-rich lysosome. Finally, TX100 resulting from chemical/enzymatic hydrolysis of CPDCs should destabilize the endosomal/endolysosomal membrane and provoke release of its content into the cytosol. DOPC as the other hydrolysis product recovers a zwitterionic structure and does not retain affinity for the nucleic acid payload. Intracellular hydrolysis (acid- or enzyme-catalyzed) of the CPDCs should thus address the two main issues in nucleic acid delivery that are cytosolic release of the payload and its dissociation from the carrier. Considering this last point, the CPDCs presented herein may be paralleled to the charge-reversal amphiphile developed by Grinstaff et al.[61-63] and might be appointed deciduous-charge amphiphiles.

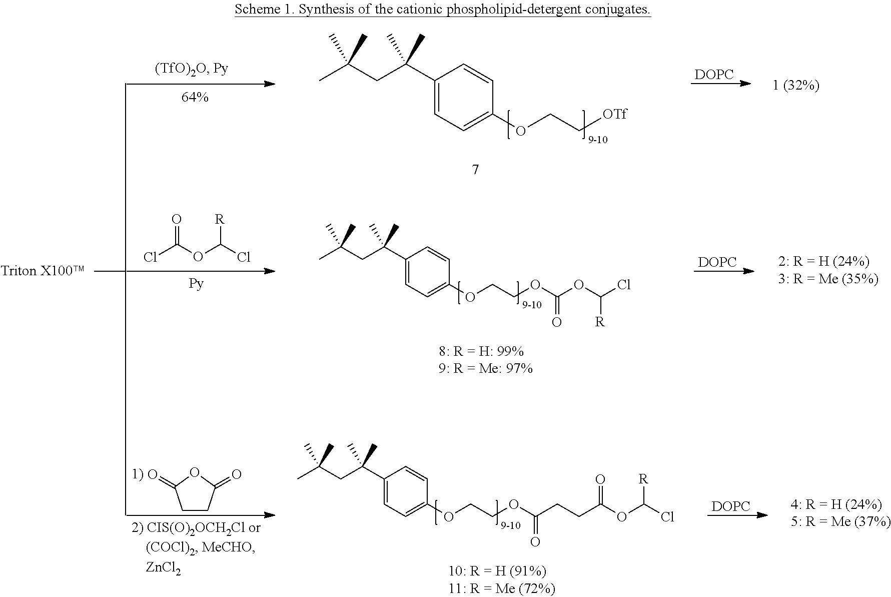

Conjugate compounds 1-5 were prepared as described in scheme 1. Detergent activation with trifluoromethanesulfonyl anhydride provided the corresponding sulfonyl ester. That compound is unstable and decomposes in a few hours on standing at room temperature in chloroform. Analysis of degradation products is consistent with a deoligomerization process leading to the formation of dioxane as reported previously with other PEG derivatives.[64-65] Consequently, the sulfonyl ester was directly submitted to nucleophilic displacement by the anionic phosphate in DOPC. Conjugate 1 was obtained in 32% yield and unreacted DOPC (61%) was recovered after purification.

Similarly, the reaction products of TX100 with chloromethyl chlorocarbonate and 1-chloroethyl chlorocarbonate (8 and 9, respectively) were reacted with DOPC to afford the cationic lipid conjugates 2 and 3, respectively, as the chloride salts. Together with recovered DOPC (55° A) and 58%, respectively), a trace amount (4-7%) of phosphodiesters resulting from cleavage of the choline moiety on compounds 2 and 3 was isolated. That is presumably to parallel with monodealkylation reactions of phosphorus esters with iodide[66] or cyanide ions,[67-69] lower nucleophilicity of the chloride ion being partly compensated for by proper positioning imposed by the neighbor ammonium tether.

Conjugates 4 and 5 were prepared following two different routes. Succinic anhydride was reacted with TX100 to produce the corresponding hemisuccinate quantitatively. The latter was reacted with chloromethylchlorosulfate[70] and precursor 10 was obtained in 91% yield. Compound 10 was then reacted with DOPC to yield CPDC 4 (24%; recovered DOPC: 47%). Besides, TX100 hemisuccinate was quantitatively transformed into the corresponding acyl chloride with (COCl)₂ and was then reacted with acetaldehyde in the presence of ZnCl₂[71] to yield the acyloxyethyl chloride precursor 11 (72%). That compound was conjugated to DOPC to afford CDPCs 5 in 37% yield (recovered DOPC: 55%). The same protocol involving formaldehyde instead of acetaldehyde failed to produce the intermediate compound 10, presumably because of solubility problems met with the aldehyde.

Preparation of reference compound 6 was similar to that of 1, replacing TX100 for polyethyleneglycol monomethylether (MW 550; n=11-12) as starting material (43% over two steps).[34]

### Example 2

### Properties of Conjugate 1

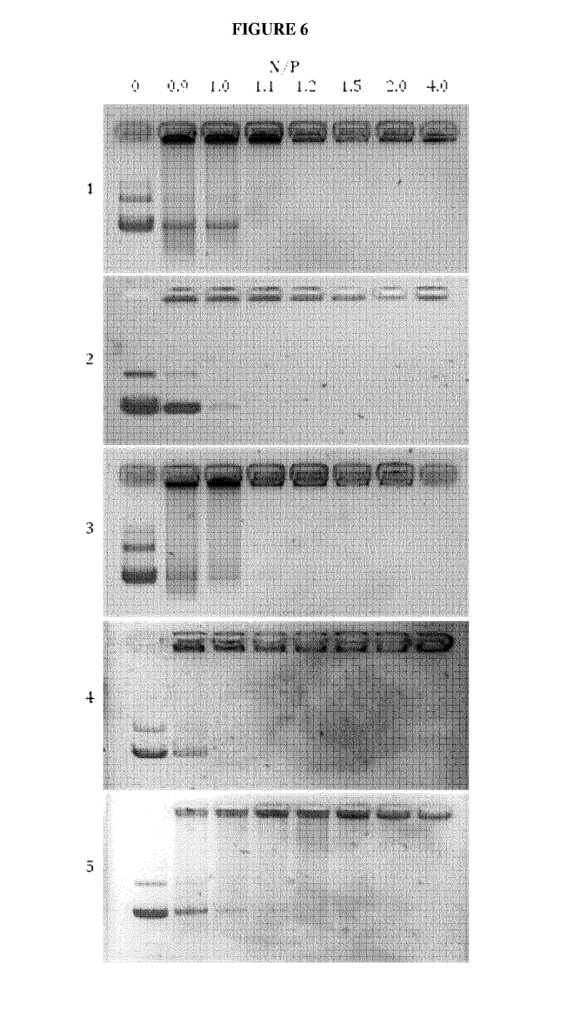

The ability of conjugate compound 1 and control compound 6 to interact electrostatically with nucleic acids was determined by electrophoretic DNA retardation assay in which a full retard of plasmid DNA is observed at a lipid/DNA phosphate ratio (N/P) corresponding to electroneutrality. The two conjugates led to a full complexation of the plasmid at an NIP of 1, indicating that the bulky substituents, TX100 or PEG block, do not dramatically perturb the electrostatic interaction between the lipid quaternary ammonium and the DNA phosphate group (FIG. 6).

Observation of siRNA/conjugate 1 complexes by transmission electron microscopy revealed that lipoplexes adopt a multilamellar organization consistent with that of some other cationic PC derivatives (FIG. 1) (R. Koynova, B. Tenchov, Top. Curr. Chem. 2010, 296, 51; D. Lleres, J. M. Weibel, D. Heissler, G. Zuber, G. Duportail, Y. Mely, J. Gene Med. 2004, 6, 415). This observation suggests that the DOPC moiety drives the overall organization of the system in the aqueous phase.

The membrane activity of compound 1 was next investigated by evaluating the release of hemoglobin from sheep erythrocytes.[48] Erythrocytes may be considered as a relevant model to investigate the ability of molecules to induce pore formation or early damage to cell plasma membranes as they cannot perform endocytosis. Furthermore, systemic administration of agents requires blood compatibility, and hemolytic activity is a useful indicator of toxicity to red blood cells.

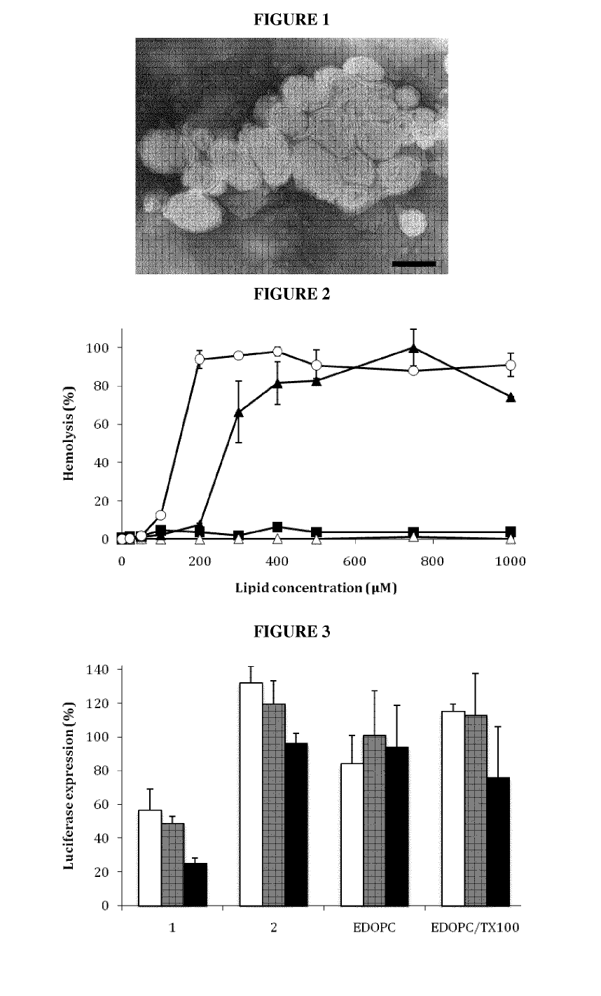

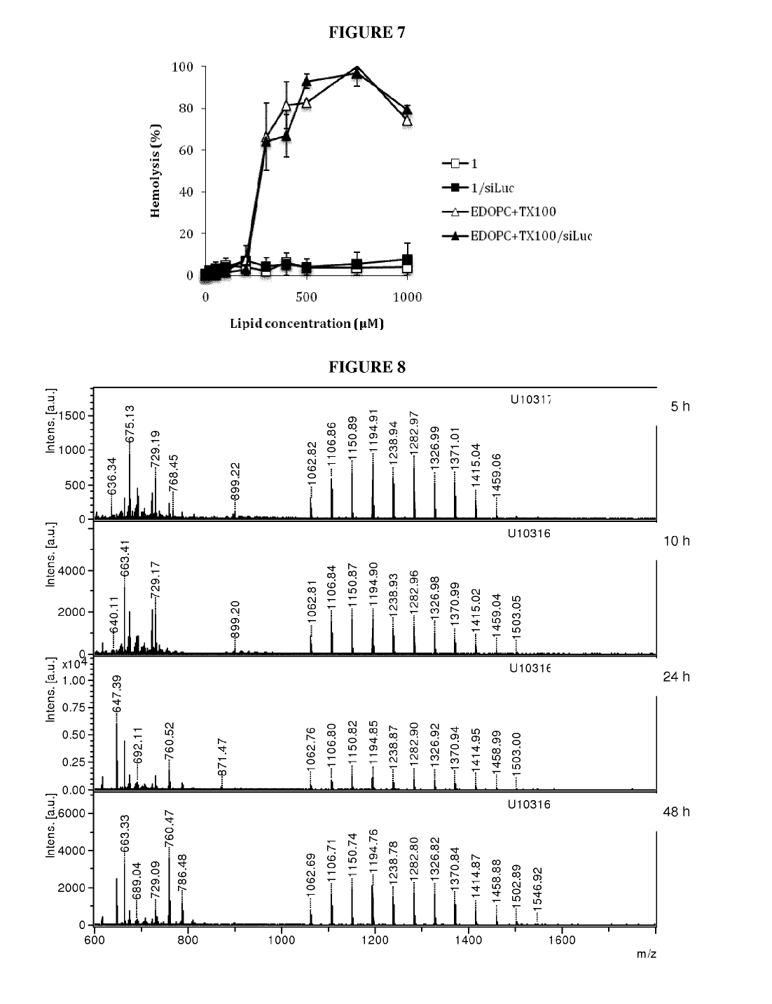

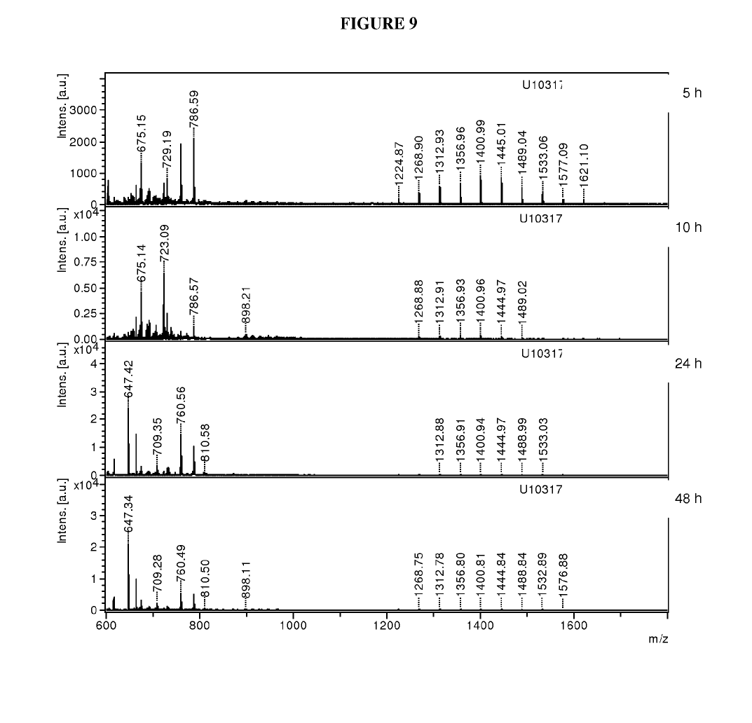

Damage to sheep erythrocytes was measured in the presence of either the compounds alone (FIG. 2) or their complex with siRNA (FIG. 7). First, TX100 achieved full membrane permeation to hemoglobin at the 200 μM concentration. Inclusion of equimolar EDOPC slightly delayed the onset of hemoglobin release but without modifying the overall hemolytic activity profile. This behavior suggests that free TX100 is the active species responsible for the erythrocyte membrane perturbation and that the fraction of detergent entrapped in EDOPC bilayers may get inactivated. Interestingly, covalent binding of TX100 to DOPC yielded an amphiphilic compound without hemolytic activity over the whole concentration range tested (20 μM to 1 mM). Besides, lipoplexes and lipids displayed similar lytic activity profile (FIG. 7). Next, the ability of the DOPC-TX100 conjugate 1 to mediate rupture of intracellular vesicles membrane was investigated by delivering to mammalian cells small interfering RNAs (siRNAs) with activity located in the cytosol.[4] The U87 epithelial-like cell line (human glioblastoma-astrocytoma) used had been stably transformed to express a luciferase gene (U87-Luc cells).[74] The delivery of a specific siRNA (siLuc) into the cytosol of U87-Luc cells promotes a sequence selective RNA interference reducing the luciferase expression and, consequently, luciferase activity, when compared to a mismatched siRNA (sic), used as a negative control. The siRNA lipoplexes were added to cells in culture medium containing 10% fetal bovine serum (FBS) and luciferase activity was measured 48 h later. The results obtained are reported in FIG. 3.

Conjugate 1 was found to efficiently assist a specific luciferase gene knockdown in U87-Luc cells since luciferase expression was silenced down to ca. 60-25% at 10-40 μM (siLuc 10 nM). PEG-DOPC conjugate 6 did not exhibit silencing activity suggesting that the 4-(1,1,3,3-tetramethylbutyl)phenyl moiety in tethered TX100 does play a key role in the transfection process. EDOPC that has been previously described as a highly efficient transfection reagent for plasmid DNA [39, 45] failed to provoke significant luciferase knockdown thus stressing the importance of the TX100 pattern in conjugate 1. Furthermore, equimolar combination of EDOPC and TX100 did not either induce a substantial silencing effect, indicating that covalent immobilization of the detergent molecule on the lipid carrier is required for efficient luciferase knockdown.

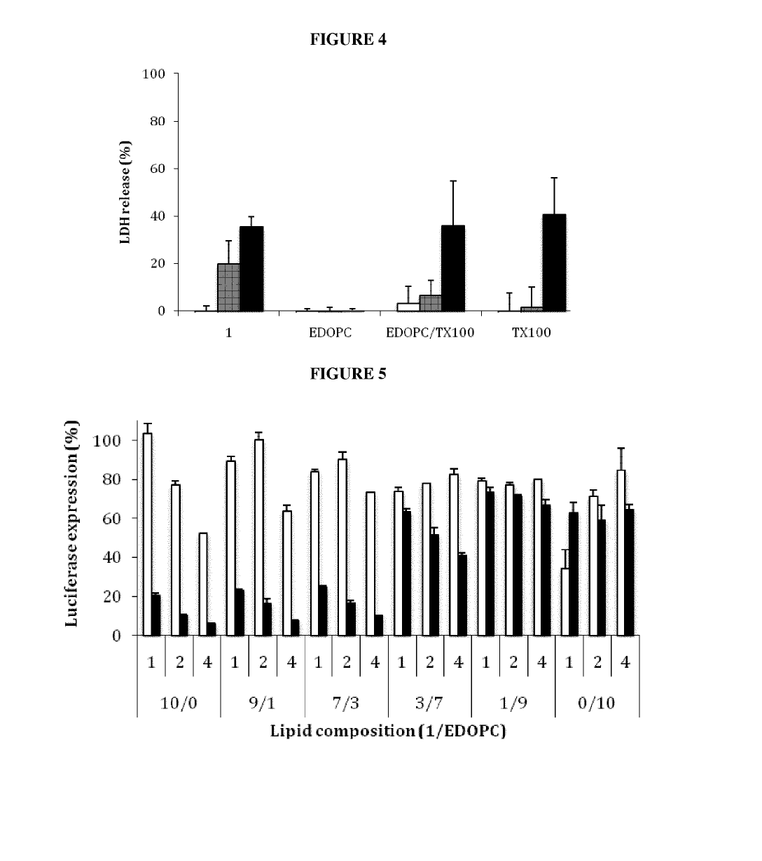

The cytotoxicity of the formulations was determined using a lactate dehydrogenase (LDH) release assay.[77] After incubation of the cells with the lipoplexes for 1 h, whatever the formulations, no significant LDH release could be measured throughout the lipid concentration range tested when compared to non-treated cells, indicating no early cell plasma membrane damage. After a 48 h incubation period, which is consistent with the siRNA delivery time schedule, LDH release remained not significant for 1/siRNA at the lower lipoplex efficient concentration, whereas some release occurred at higher concentration in a dose-dependent manner, suggesting that high transfection efficiency may ultimately result in some cell damage (FIG. 4). Accordingly, no LDH release was observed for the non-active EDOPC lipoplexes throughout the lipid concentration range tested. On the other hand, the dilution of conjugate 1 with EDOPC significantly reduced the cytotoxicity of the lipoplexes while preserving the highest luciferase silencing effect up to an EDOPC content of 30% (FIG. 5). These results suggest that cytotoxicity of conjugate 1 may be attenuated via lipid mixing with only reduced adverse effect on gene silencing efficiency.

It has been suggested that phosphotriesters may be substrates of intracellular phospholipases in endosomes and lysosomes.[78] In order to determine whether endosomolytic activity of conjugate 1 might be related to an enzyme-triggered release of the detergent TX100 inside the endocytic compartment, the fate of conjugate compound 1 was analyzed after addition to cells by mass spectrometry. Cells were treated with 1/siLuc lipoplexes as described for the siRNA delivery experiments with the difference that siRNA lipoplexes remaining in the cell culture supernatant were removed by cell washing 5 h after their addition to the cells. Cells were used thereafter or incubated for various periods of time before treatment with chloroform/methanol (2/1, v/v) to extract the total lipids from the cells. Analysis of the lipid extracts was performed by MALDI-ToF mass spectrometry (FIG. 8). Semi-quantitative interpretation of the data was realized using endogenous POPC (palmitoyl oleoyl phosphatidyl choline, m/z 760) as an internal standard though it is clear that a perturbation of the cell metabolism may have some repercussions on the lipid composition of the cell (e.g. via oxidation, hydrolysis, or esterification reactions). Nevertheless, the results obtained showed that the DOPC-TX100 conjugate 1 still was abundant in the cells after a 48 h incubation period, which is an indication that it was not massively degraded intracellularly on the time scale of siRNA delivery.

As described herein, the cationic detergent-phospholipid conjugate (TX100-DOPC conjugate 1) binds nucleic acids and does not display hemolytic activity up to 1 mM, which is above the concentration usually used in nucleic acid delivery protocols, and is a lamellar phase forming lipid. Conjugate 1 can very efficiently deliver siRNA into the cytosol of mammalian cells without the need for a helper lipid (i.e. DOPE), which is required with lamellar phase forming cationic lipids. It appears the covalent anchoring of TX100 to the cationic lipid carrier as well as the dangling hydrophobic part of the detergent moiety assist in ensuring siRNA delivery efficiency of the compound. The absence of intracellular degradation of the TX100-DOPC conjugate supports that delivery of siRNA into the cytosol relies on a unique intrinsic endosomolytic activity of that lamellar phase forming lipid and is not due to detergent release in the intracellular compartment resulting from enzymatic hydrolysis. Therefore, conjugate 1 may be regarded as a dissymmetrical bolaamphiphilic compound with improved capacity for fusion with membranes. One hypothesis for this activity is that, due to hydrophilicity and flexibility of the ethyleneoxide oligomer spacer, the dangling hydrophobic part of TX100 (tetramethylbutylphenyl moiety) would explore the hydrophobic neighborhood within the endosome and moor the particle with the endosome membrane. The compound would literally act as a “grapnel lipid” and might trigger lipid fusion, resulting in siRNA decondensation and release into the cytosol.

### Example 3

### Hydrolytic Properties of the Conjugates

The hydrolytic stability of CPDCs/conjugates under neutral and acidic conditions was evaluated in a model experiment involving ³¹P-NMR measurements. Phosphotriesters and phosphodiesters display ³¹P chemical shifts differing by 5-6 ppm allowing a precise monitoring of the hydrolysis reaction. CPDCs/conjugates were formulated into liposomes using an injection technique.[47] Liposomes were prepared at pH 7.4 and pH 4.5, and incubated at 37° C. and hydrolysis was monitored by ³¹P-NMR measurements. Periodical acquisition of ³¹P-NMR spectra allowed determination of the time t_{1/2} required for 50% hydrolysis (Table 2). Time required for 50% hydrolysis (t_{1/2}) was calculated from the theoretical curve fitting with the experimental data.

TABLE 2
Hydrolytic stability of CPDCs.

Compound

| | | 1* | 2 | 3 | 4 | 5 | 6* |
|---|---|---|---|---|---|---|---|
| t_{1/2} (h) | pH 7.4 | — | 84 | 25 | 122 | 9.4 | — |
| | pH 4.5 | — | 615 | 52 | 245 | 7.2 | — |

*No hydrolysis observed after a 14 days incubation period.

As anticipated, the cationic conjugate compound 1 retained a full integrity both under neutral and acidic conditions over an extended period of time (>14 d). The same behavior was observed for reference compound 6. The hydrolysis rate of phosphoryloxymethylcarbonates 2 and 3, and phosphoryloxymethylester 4 was slowed down under acidic conditions by ca. a twofold factor or more. For compound 5 only, hydrolysis was quicker at pH 4.5 than at pH 7.4. That clearly indicates that these results cannot be analyzed just taking into account the chemical reactivity of the acetal moiety but should be interpreted with regard to the respective sensitivity of the carbonate and ester moieties towards hydrolysis in aqueous solutions depending on the reaction mechanism (specific base catalyzed, water catalyzed, or specific acid catalyzed).[72, 73] Alkyl substitution on the acetal carbon atom invariably provoked an increase in the hydrolysis rate, both under basic and acidic conditions.

### Example 4

### CPDC/siRNA Nanoparticle Formation and Characterization

The ability of CPDCs to interact electrostatically with nucleic acids was studied by conventional electrophoretic DNA retardation assays in which a full retard of plasmid DNA is observed at a lipid/DNA phosphate ratio (N/P) corresponding to electroneutrality. The five cationic Triton X-100 conjugates led to a full complexation of the plasmid at N/P 1, indicating that the detergent block does not impact on the ability of the cationic head group to interact with the DNA phosphate groups (FIG. 6). The same holds for compound 6. The size of siRNA complex with 1-6 as determined by light scattering experiments was in the range 100-300 nm which is consistent with observation by transmission electron microscopy.

### Example 5

### Gene Knockdown and Cell Viability Assays

Transfection efficiency of compounds 1-6 was investigated using the protocol described above. The results of the gene silencing experiments indicated that compounds 1-3 may efficiently assist a specific luciferase gene knockdown in U87-Luc cells since luciferase expression was silenced down to ca. 30-15% in a dose-dependent manner (FIG. 14). The phosphoryloxymethylsuccinate compound (conjugate 4) displayed intermediate luciferase knockdown activity (luciferase expression reduced to 66%) whereas the corresponding phosphoryloxy-1-ethylsuccinate (compound 5) proved inactive over the whole concentration range investigated (1-4 nmol of lipid per well which corresponds to 10-40 μM). The same was observed with conjugate 6, which supports that the terminal hydrophobic moiety in DOPC-TX100 conjugates (i.e. the 4-(1,1,3,3-tetramethyl)butyl phenyl group) is essential for transfection efficiency. Besides, EDOPC has a phosphotriester backbone closely related to that of the title compounds but revealed inefficient to knockdown luciferase expression. This further supports that the TX100 moiety on the phosphocholine head group is strongly required for transfection efficiency. Equimolar combination of EDOPC and TX100 did not induce substantial silencing effect either, indicating that covalent immobilization of the detergent molecule on the lipid carrier is necessary for efficient luciferase knockdown. In the same experimental setup, DOTAP, a benchmark cationic lipid in DNA transfection, was unable to assist siRNA silencing activity.

In vitro lipoplexes cytotoxicity was then assessed by cell metabolic activity measurements using a tetrazolium-based colorimetric assay (MTT assay) (FIG. 15).[76] The experiments were realized on the 16HBE cell line. The cationic phosphotriester EDOPC was well tolerated by cells and did not significantly impair mitochondrial activity. Incorporation of a molar equivalent of TX100 in the formulation did not induce adverse effect at the lower concentrations tested (10 and 20 μM) but revealed fully lethal to cells at higher concentration (40 μM). The same was observed with TX100 alone (i.e. in the absence of EDOPC) indicating that TX100 20-40 μM was enough to decrease cell viability. Interestingly, conjugate 1 and carbonates 2 and 3 with quite similar transfection efficiency displayed different cytotoxicity profiles. Whereas conjugate 1 markedly impaired mitochondrial activity at 20 μM, carbonate analogs were much better tolerated as ca. 70% of the cell metabolic activity was maintained at the higher concentration of these lipids (40 μM). On the other hand, the two succinate derivatives 4 and 5 were non-cytotoxic since no significant perturbation of cell metabolic activity was observed at the highest concentration tested.

Cells treated with chemicals or nanoparticles sustain stress. This stress may result in a reduction of mitochondrial activity that may be only transient and is not necessarily associated with cell death. Consequently, the tetrazolium-based viability assays such as MMT assay fail to give accurate data on cell survival. On the other hand, cell death by necrosis is accompanied by damage to cell plasma membrane and the subsequent release of cytoplasmic material into the extracellular space. Thus, loss of cell viability can be directly monitored by measuring lactate dehydrogenase (LDH) activity in the extracellular compartment. Cytotoxic effects after treatment of cells with lipoplexes were thus investigated using a LDH release assay.[77]

After incubation of the cells with the lipoplexes for 1 h, whatever the formulations, no significant LDH release could be measured throughout the lipid concentration range tested when compared to non-treated cells, indicating no early damage to cell plasma membrane. After a 48 h incubation period, which is consistent with the siRNA delivery time schedule, LDH release occurred in a dose-dependent manner for the 1/siRNA complex though it was not significant at the lower concentration (FIG. 16). The carbonate based complexes (2/siRNA and 3/siRNA) revealed less cytotoxic as higher lipid concentration provoked less than 10% LDH release over the considered period. In the succinate series (4/siRNA and 5/siRNA) no significant toxicity was measured, as was practically the case for EDOPC and, to a lesser extent, for DOTAP. In contrast, formulations incorporating free TX100 (alone or mixed with EDOPC) drastically impaired cells as more than 50% LDH release was measured with the higher dose tested (4.0 nmol, which corresponds to 40 μM).

The membrane activity of the conjugates was next investigated by evaluating the release of hemoglobin from sheep erythrocytes. In a preliminary experiment (see Example 2), damage to sheep erythrocytes was measured in the presence of two selected lipid formulations (1, EDOPC+TX100), with and without siRNA. As might be expected considering the large excess of cationic lipid used to form the complexes with siRNA (N/P ranging from 25 to 100), a lipid and the corresponding lipoplexes displayed the same hemolytic behavior (FIG. 7).

Thus the following experiment was exclusively conducted with lipids, and lipoplexes were omitted (FIG. 17). Less active conjugates in transfection (4 and 5) were not evaluated either. The results obtained indicate that conjugate 1 was devoid of hemolytic activity in the concentration range tested (0-1 mM) whereas carbonate analogs 2 and 3 started provoking membrane permeation at 50 μM. Interestingly, the destabilizing effect promoted by these two lipids reached a plateau at a concentration of ca. 200 μM and hemolysis did not proceed beyond 50%. EDOPC behaved similarly to conjugate 1 and proved inactive to permeate hemoglobin through the erythrocyte membrane. Inclusion of equimolar TX100 however drastically injured the cells and full hemoglobin permeation was obtained at ca. 400 μM.

TX100 alone provoked full membrane destabilization at lower concentration (150-200 μM) which suggests that the fraction of detergent entrapped in EDOPC bilayers may get inactivated. Over the hemolysis experiment time scale (2 h), we may assume that the hydrolytic cleavage of the CPDCs outside cells should come to very little (<5-10%). Consequently, released TX100 in the extracellular compartment cannot fully explain the hemolytic behavior of the compounds. This one should then result from some participation of the non degraded conjugates. On the other hand, fusion events between the lipid particles and the membrane of erythrocytes might lead to exposure of CPDCs to intracellular enzymes. This would result in enhanced degradation (vide infra) with possible intracellular release of TX100, finally provoking permeation of the erythrocytes membrane. The hypothesis of “grapnel lipids” would account for fusion capabilities of the CPDCs.

The DOPC-TX100 conjugates were designed so they may be metabolized inside cells, either chemically under various pH conditions or being substrate of enzymes, especially phospholipases (PL). MacDonald et al. previously demonstrated that EDOPC is hydrolyzed by purified PLA₂ (cobra and bee venom), is only slowly metabolized by PLD (cabbage), and is resistant to PLC (Clostridium).[78] Using a fluorescent analog of EDOPC, intracellular hydrolysis of the compound at C² could be evidenced only after an incubation period of 3 days revealing a very slow kinetics for the enzyme-mediated degradation. Therefore it has been proposed that lipid degradation is limited by accessibility, the very mass of the lipid presenting a significant barrier to access by intracellular lipases.

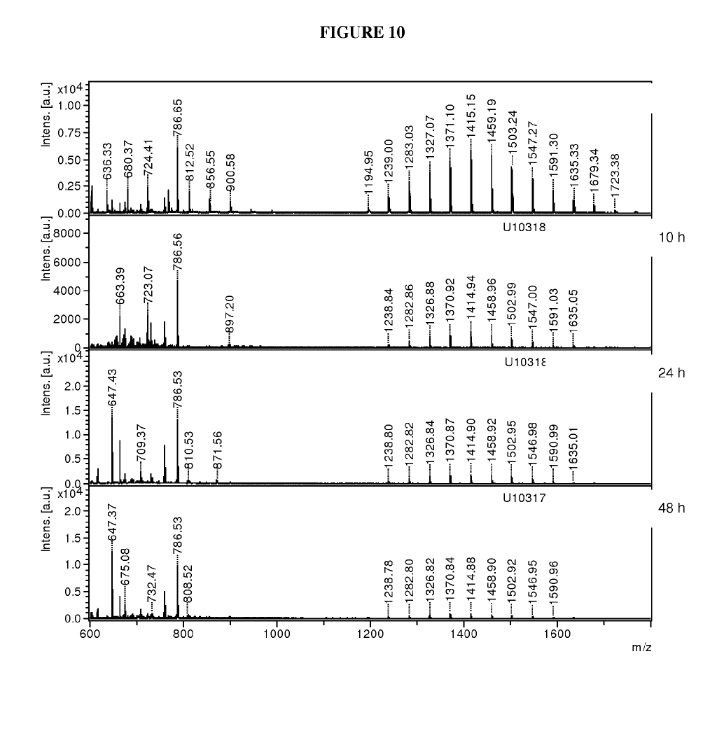

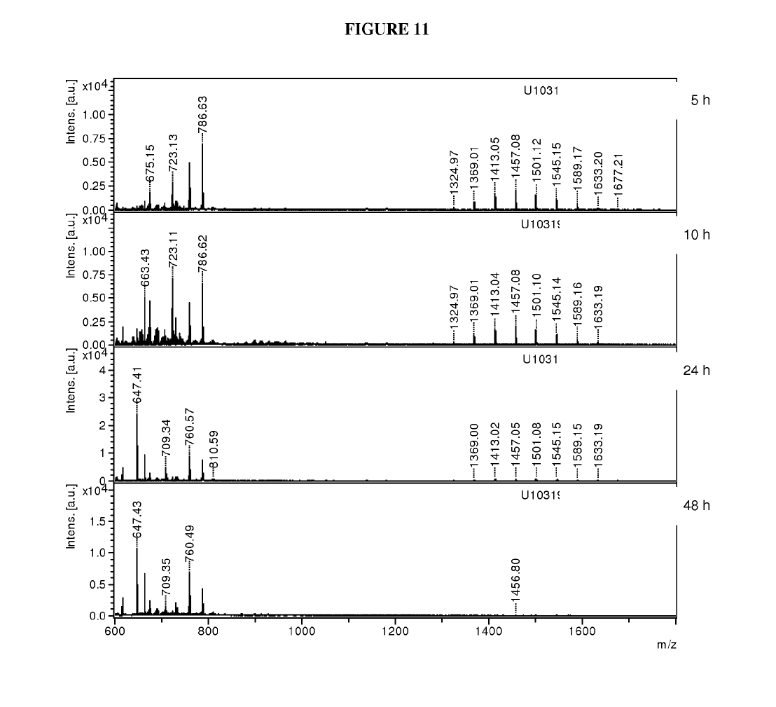

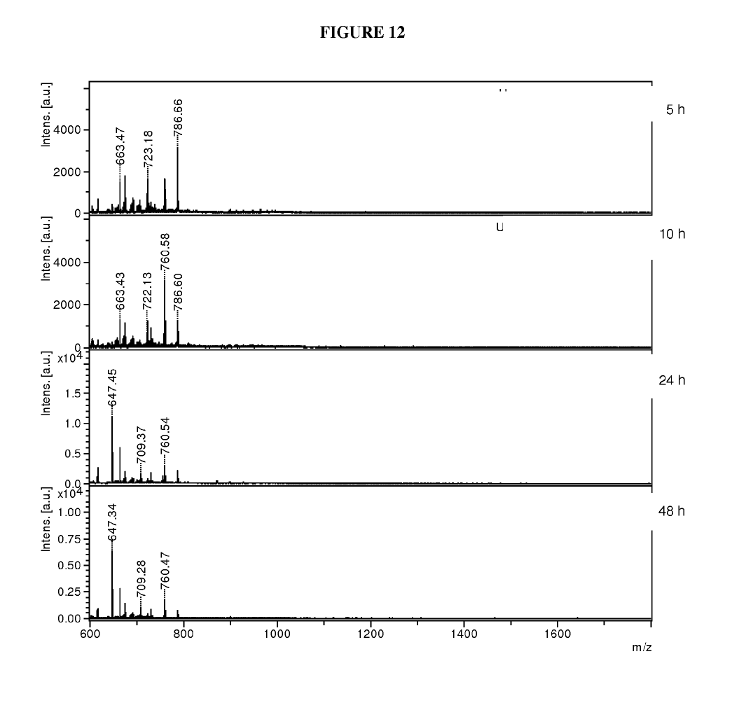

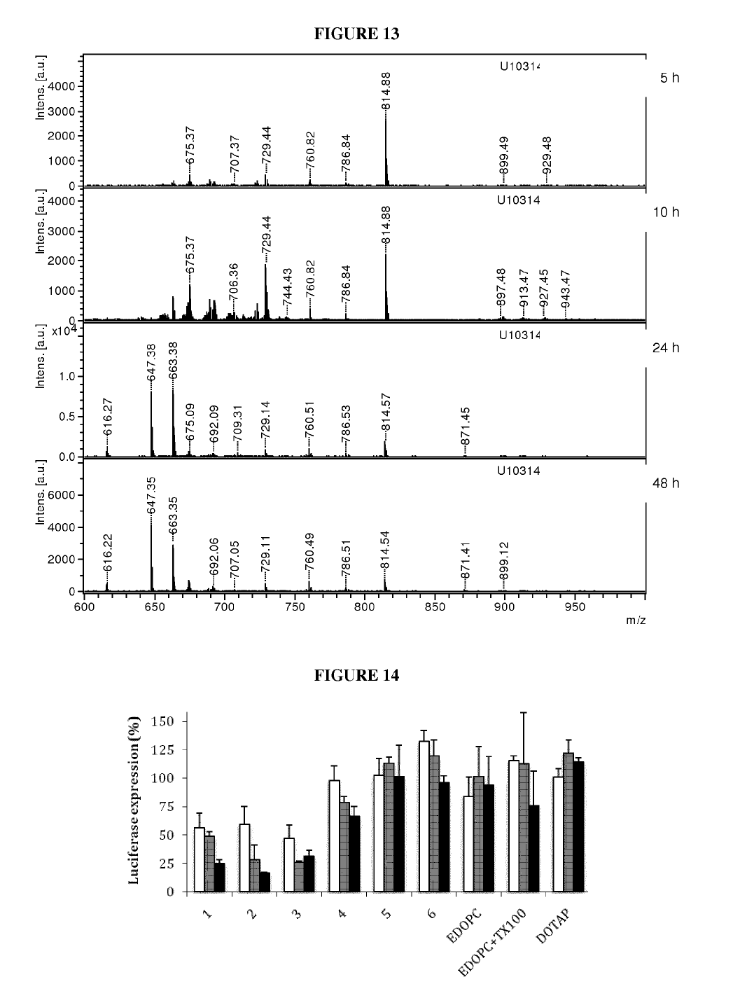

In order to determine whether endosomolytic activity of DOPC-TX100 conjugates might be related to their degradation with a possible release of detergent inside the endocytic compartment, the fate of compounds 1-5 after addition to cells was analyzed by mass spectrometry. Cells were treated with siLuc lipoplexes as described for the siRNA delivery experiments with the difference that siRNA lipoplexes remaining in the cell culture supernatant were removed by cell washing 5 h after their addition to the cells. Cells were used thereafter or incubated for various periods of time before treatment with chloroform/methanol (2/1, v/v) to extract the total lipids. Analysis of the lipid extracts was performed by MALDI-ToF mass spectrometry (FIGS. 8-13).

Though the chemical hydrolysis of the labile conjugates consistently produced DOPC as was supported by the previous ³¹P-NMR experiments (see Example 2), the enzyme-mediated hydrolysis potentially may lead to the formation of a series of products. Except the starting DOPC-TX100 conjugates, no other PEGylated compound could be extracted in the organic phase. This indicates that the conjugates were not enzymatically metabolized into the corresponding 1,2-dioleoyl-sn-glycero-3-phosphatidic acid-TX100 conjugates (extractable) with production of choline as a consequence of PLD activity. Also this is consistent with the high water solubility of TX100 and that of the putative lysoPC-TX100 conjugates (resulting from activity of PLA₁ and PLA₂) which are not extractable from the aqueous phase. The lipid composition in cells being subjugated to various regulation pathways, the amount of DOPC in the extracts was not under the sole control of the hydrolysis of the conjugates and thus could not be used to monitor the hydrolysis.

Semi-quantitative analysis of the cell lipid extracts was then realized using endogenous POPC (palmitoyl-oleoyl-sn-glycero-3-phosphatidyl choline, m/z 760) as an internal standard and the amount of residual conjugates was roughly evaluated. The results obtained clearly indicated that conjugate 5 was rapidly hydrolyzed in cells and could not be detected even after the shorter incubation time (5 h). That was fully consistent with the low hydrolytic stability previously evidenced by NMR. Compounds 2 and 4 were readily metabolized as well and could be hardly detected after 10-24 h whereas a small residue of compound 3 still could be detected after a 48 h incubation period. On the other hand, conjugate 1 and EDOPC displayed similar hydrolysis profile and the two compounds still remained abundant after an incubation with cells for 48 h. These results clearly evidence that the conjugates are much more quickly metabolized inside cells than was expected from the chemical hydrolysis experiments. Furthermore, there is no obvious correlation between degradation kinetics and transfection efficiency.

Conjugate 5 appeared especially labile and likely did not condense or protect siRNA long enough to favor cell entry or avoid degradation by nucleases before reaching its target. That is supported by the results obtained in the transfection experiments. Though conjugates 2 and 4 were metabolized with similar rates, they offered various transfection efficiencies. On the other hand, conjugates 1 and 2 that provided similar transfection efficiencies displayed a quite different metabolization profile, and EDOPC that could compare with 1 with respect to metabolic stability was inactive in siRNA transfection. Considering cytotoxicity, a general trend might be observed and the more readily degraded conjugates indeed were those with lower toxicity (see MTT and LDH assays).

### B. Part 2

### Compounds of the Invention Wherein R⁸ is an Alkyl Chain

The aim of this study is to assess the impact of the length of R8 alkyl chain and the nature of hydrolysable connector on transfection activity and cytotoxicity.

### Materials and Methods

1. Materials: See Materials of PART 1 Hereabove.

Moreover, pCMV-Luc expression plasmid (BD Biosciences Clontech, Franklin Lakes, N.J., USA) was used as reporter gene to monitor transfection activity. This plasmid encoded the firefly luciferase gene under the control of a strong promoter. The small double stranded siRNAs were from Eurogentec (Angers, France). BHK-21 cells (Syrian hamster kidney cells), Calu-3 cells (epithelial lung adenocarcinoma, HBT-55), U87 cells (glioblastoma, astrocytoma cell line derived from human malignant glioma; HTB-14), A549 cells (human lung carcinoma; CCL-185), and NCI-H292 cells (human lung mucoepidermoid carcinoma; CRL-1848) were obtained from ATCC-LGC (Molsheim, France). U87 and A549 cell lines were transformed to stably express the Photinus pyralis luciferase gene originating from the pGL3 plasmid (Clontech, Mountain View, Calif.). The plasmid coded as well for a resistance gene to G418, and the resulting U87Luc and A549Luc cells were thus selected on this selective antibiotic.

2. Synthesis

The following compounds were prepared. These compounds correlate with compound of formula (IV) comprising a hydrolysable connector L without oligoethylene glycol spacer.

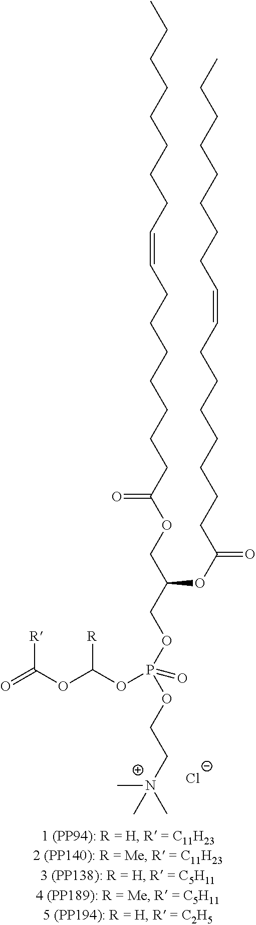

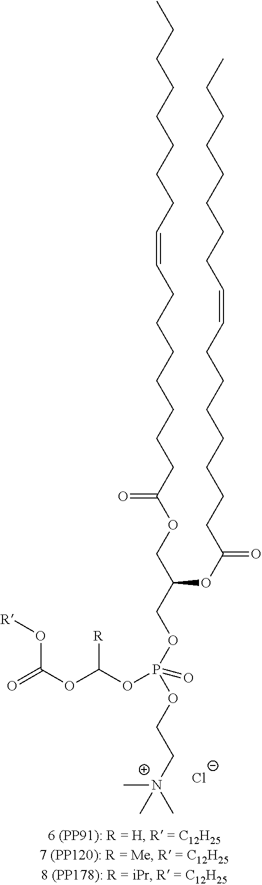

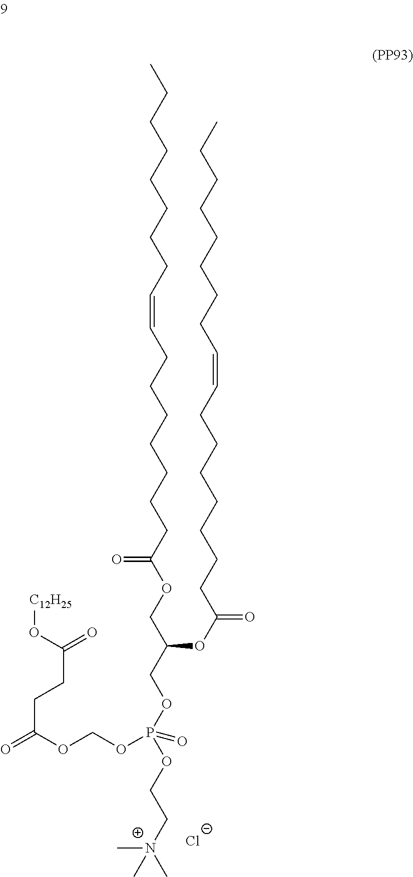

These compounds were synthetized upon the following synthetic route:

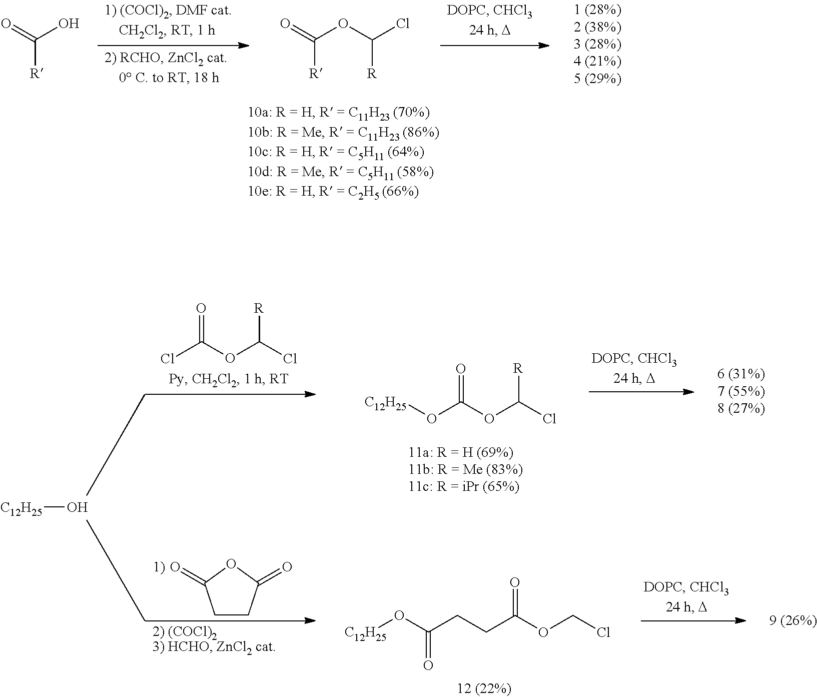

### 2.1 Preparation of Chloroalkyl Esters (10a-10e)

### Chloromethyl Dodecanoate (10a)

Dodecanoyl chloride (3.30 g, 15.1 mmol) was added slowly to a mixture of paraformaldehyde (0.45 g, 15.1 mmol) and a catalytic amount of anhydrous ZnCl₂ (41 mg, 0.3 mmol) at −10° C. under an argon atmosphere. The reaction mixture was stirred at this temperature for 1 h, and at room temperature for an additional period of 18 h. The crude reaction mixture was directly purified by flash column chromatography over silica gel (PE/Et₂O 95:5) to yield compound 10a (2.60 g, 70%) as a clear oil. TLC R_{f} 0.40 (PE/Et₂O 95:5). ¹H-NMR (400 MHz, CDCl₃) δ 5.67 (s, 2H), 2.34 (t, ³J_{HH}=7.5 Hz, 2H), 1.62 (m, 2H), 1.31-1.22 (m, 16H), 0.84 (t, ³J_{HH}=6.7 Hz, 3H). ¹³C-NMR (100 MHz, CDCl₃) δ 171.8, 68.7, 34.2, 32.1, 29.7 (2C), 29.6, 29.5, 29.3, 29.1, 24.7, 22.8, 14.3.

### 1-Chloroethyl Dodecanoate (10b)

Compound 10b (4.05 g, 86%) was obtained as a clear oil from dodecanoyl chloride, acetaldehyde and ZnCl₂ following the same procedure as described for the preparation of 10a. TLC R_{f} 0.40 (PE/Et₂O 95:5). ¹H-NMR (400 MHz, CDCl₃) δ 6.53 (q, ³J_{HH}=5.8 Hz, 1H), 2.32 (t, ³J_{HH}=7.4 Hz, 2H), 1.76 (d, ³J_{HH}=5.8 Hz, 3H), 1.61 (m, 2H), 1.32-1.23 (m, 16H), 0.86 (t, ³J_{HH}=6.9 Hz, 3H). ¹³C-NMR (100 MHz, CDCl₃) δ 171.6, 34.4, 32.1, 29.8 (2C), 29.7, 29.6, 29.5, 29.4, 29.2, 25.4, 24.8, 22.9, 14.3.

### Chloromethyl Hexanoate (10c)

Compound 10c (2.60 g, 64%) was obtained as a clear oil from hexanoyl chloride, paraformaldehyde and ZnCl₂ following the same procedure as described for the preparation of 10a. TLC R_{f} 0.25 (PE/Et₂O 98:2). ¹H-NMR (300 MHz, CDCl₃) δ 5.68 (s, 2H), 2.36 (t, ³J_{HH}=7.2 Hz, 2H), 1.64 (m, 2H), 1.34-1.23 (m, 4H), 0.88 (t, ³J_{HH}=6.2 Hz, 3H). ¹³C-NMR (100 MHz, CDCl₃) δ 171.9, 69.8, 34.2, 31.3, 24.4, 22.4, 14.1.

### 1-Chloroethyl Hexanoate (10d)

Compound 10d (3.35 g, 58%) was prepared from hexanoyl chloride, acetaldehyde and ZnCl₂ following the same procedure as described for the preparation of 10a. TLC R_{f} 0.20 (PE/Et₂O 98:2). ¹H-NMR (400 MHz, CDCl₃) δ 6.53 (q, ³J_{HH}=5.8 Hz, 1H), 2.32 (dd, ³J_{HH}=7.2 and 8.5 Hz, 2H), 1.76 (d, ³J_{HH}=5.8 Hz, 3H), 1.63 (m, 2H), 1.32-1.27 (m, 4H), 0.87 (t, ³J_{HH}=6.4 Hz, 3H). ¹³C-NMR (100 MHz, CDCl₃) δ 171.7, 80.8, 34.3, 31.3, 25.4, 24.4, 22.4, 14.1.

### Chloromethyl Propionate (10e)

Compound 10e (2.20 g, 66%) was prepared from propionyl chloride, paraformaldehyde and ZnCl₂ following the same procedure as described for the preparation of 10a. TLC R_{f} 0.10 (PE/Et₂O 98:2). ¹H-NMR (300 MHz, CDCl₃) δ 5.68 (s, 2H), 2.39 (q, ³J_{HH}=7.5 Hz, 2H), 1.15 (t, ³J_{HH}=7.5 Hz, 3H). ¹³C-NMR (100 MHz, CDCl₃) δ 172.6, 68.8, 27.6, 8.8.

### 2.2. General Preparation of Chloroalkyl Carbonates (11a-11c)

### Chloromethyl Dodecyl Carbonate (11a)

Dodecanol (3.00 g, 16.0 mmol) and anhydrous Et₃N (4.46 mL, 32.0 mmol) were dissolved in dry CHCl₃ (50 mL). The resulting mixture was cooled to 0° C. under argon and chloromethylchloroformate (2.21 mL, 16.0 mmol) was added dropwise. The reaction mixture was stirred at room temperature for 1 h. Then it was diluted with CH₂Cl₂ (50 mL) and washed 2 times with 1M NaHCO₃, and with brine. The organic layer was dried over MgSO₄ and concentrated under reduced pressure. The residue was purified by flash column chromatography over silica gel (PE/Et₂O 95:5) to yield 11a (3.10 g, 69%) as a clear oil. TLC R_{f} 0.50 (PE/Et₂O 95:5). ¹H-NMR (400 MHz, CDCl₃) δ 5.71 (s, 2H), 4.20 (t, ³J_{HH}=6.7 Hz, 2H), 1.67 (m, 2H), 1.35-1.23 (m, 18H), 0.86 (t, ³J_{HH}=6.7 Hz, 3H). ¹³C-NMR (100 MHz, CDCl₃) δ 153.6, 72.3, 69.6, 32.1, 29.8 (2C), 29.7, 29.6, 29.5, 29.3, 28.7, 25.8, 22.9, 14.3.

### 1-Chloroethyl Dodecyl Carbonate (11b)

Compound 11b (2.45 g, 83%) was obtained as a clear oil from dodecanol, pyridine and chloroethylchloroformate, following the same procedure as described for the preparation of 11a. TLC R_{f} 0.54 (PE/Et₂O 95:5). ¹H-NMR (300 MHz, CDCl₃) δ 6.40 (q, ³J_{HH}=5.8 Hz, 1H), 4.17 (t, ³J_{HH}=6.7 Hz, 2H), 1.80 (d, ³J_{HH}=5.8 Hz, 3H), 1.68-1.63 (m, 2H), 1.33-1.23 (m, 18H), 0.85 (t, ³J_{HH}=6.3 Hz, 3H). ¹³C-NMR (100 MHz, CDCl₃) δ 153.1, 84.7, 69.3, 32.1, 29.8, 29.7, 29.6, 29.5, 29.3, 28.7, 25.8, 25.4, 22.9, 14.3.

### 1-Chloro-2-methylpropyl dodecyl carbonate (11c)

Compound 11c (2.08 g, 65%) was obtained as a clear oil from dodecanol, pyridine and 1-chloro-2-methylpropyl chloroformate, following the same procedure as described for the preparation of 11a. TLC R_{f} 0.62 (PE/Et₂O 95:5). ¹H-NMR (400 MHz, CDCl₃) δ 6.16 (d, ³J_{HH}=4.6 Hz, 1H), 4.18 (dt, ³J_{HH}=3.4 and 6.7 Hz, 2H), 2.18 (dhept, ³J_{HH}=4.6 and 6.7 Hz, 1H), 1.68-1.65 (m, 2H), 1.34-1.24 (m, 18H), 1.05 (d, ³J_{HH}=6.7 Hz, 6H), 0.86 (t, ³J_{HH}=6.9 Hz, 3H). ¹³C-NMR (100 MHz, CDCl₃) δ 153.6, 92.9, 69.3, 35.5, 29.8 (2C), 29.7, 29.6, 29.5, 29.3, 28.7, 25.9, 25.8, 22.9, 17.4 (2C), 14.3.

### Chloromethyl Dodecyl Succinate (12)

Dodecanol (5.0 g, 26.8 mmol), DMAP (163 mg, 1.34 mmol) and succinic anhydride (3.22 g, 32.21 mmol) were stirred in anhydrous toluene (50 mL) and the resulting reaction mixture was refluxed for 48 h under argon. The mixture was cooled down, extracted with brine, dried over MgSO₄, and concentrated under reduced pressure. The residue was purified by flash column chromatography over silica gel eluting first with PE/Et₂O 70:30 to remove unreacted dodecanol, and finally with CH₂Cl₂/MeOH 95:5 to obtain 4-(dodecyloxy)-4-oxobutanoic acid (3.25 g, 43%). TLC R_{f} 0.50 (CH₂Cl₂/MeOH) 95:5). ¹H-NMR (400 MHz, CDCl₃) δ 4.07 (t, ³J_{HH}=6.8 Hz, 2H), 2.67-2.58 (m, 4H), 1.59 (m, 2H), 1.32-1.23 (m, 18H), 0.86 (t, ³J_{HH}=6.9 Hz, 3H). ¹³C-NMR (100 MHz, CDCl₃) δ 178.0, 172.4, 65.3, 32.1, 29.8 (2C), 29.7 (2C), 29.6, 29.4, 29.1 (2C), 28.7, 26.1, 22.9, 14.3. A solution of the latter compound (3.25 g, 11.3 mmol) in anhydrous CH₂Cl₂ (50 mL) was treated with oxalyl chloride (1.10 mL, 12.5 mmol) at 0° C. under an argon atmosphere. After 30 minutes, the reaction mixture was allowed to warm to room temperature and was stirred for an additional period of 1 h. Volatiles were removed under reduced pressure, and the residue was coevaporated twice with anhydrous toluene. The expected acyl chloride derivative was recovered in quantitative yield and used in the next step without further purification. To dodecyl 4-chloro-4-oxobutanoate (275 mg, 0.90 mmol) were successively added paraformaldehyde (27 mg, 0.90 mmol) and anhydrous ZnCl₂ (2.5 mg, 2% mol). The reaction mixture was heated at 35° C. under an argon atmosphere for 18 h. After cooling to room temperature, the residue was purified by flash column chromatography over silica gel (PE/Et₂O 80:20) to yield compound 12 (150 mg, 50%) as a clear oil. TLC R_{f} 0.42 (PE/Et₂O 80:20). ¹H-NMR (400 MHz, CDCl₃) δ 5.69 (s, 2H), 4.07 (t, ³J_{HH}=6.9 Hz, 2H), 2.70-2.62 (m, 4H), 1.61-1.58 (m, 2H), 1.28-1.24 (m, 18H), 0.86 (t, ³J_{HH}=6.7 Hz, 3H). ¹³C-NMR (100 MHz, CDCl₃) δ 172.1, 170.7, 68.9, 65.4, 32.1, 29.8 (2C), 29.7 (2C), 29.6, 29.5, 29.2, 28.9, 28.8, 26.1, 22.9, 14.3.

### 2.3. General Procedure for the Preparation of Cationic Lipids

To a solution of DOPC in anhydrous CHCl₃ was added a solution of the freshly prepared electrophiles 10a-e, 11a-c or 12 (8 eq.) in CHCl₃. The resulting reaction mixture was refluxed for 18 h under an argon atmosphere. Then it was cooled down at room temperature and concentrated under reduced pressure. Analytically pure compounds were obtained after purification by flash chromatography over silica gel (CH₂Cl₂/MeOH 98:2 to 90:10).

### 1,2-Dioleoyl-sn-glycero-3-[(dodecanoyloxy)methyl]phosphocholine chloride (1) (Also Called PP94)

Compound 1 (mixture of two diastereomers, 110 mg, 28%) was obtained as a waxy solid from DOPC and 10a in CHCl₃ (5 mL). TLC R_{f} 0.20 (CH₂Cl₂/MeOH 90:10). ¹H-NMR (400 MHz, CDCl₃) δ 5.67-5.56 (m, 2H), 5.36-5.28 (m, 4H), 5.23 (br s, 1H), 4.57 (br s, 2H), 4.32-4.11 (m, 6H), 3.49 (s, 9H), 2.39-2.27 (m, 6H), 1.97 (m, 8H), 1.62-1.57 (m, 6H), 1.27-1.23 (m, 56H), 0.85 (t, ³J_{HH}=6.6 Hz, 9H). ¹³C-NMR (100 MHz, CDCl₃) δ 173.4, 173.0, 172.5, 130.2, 129.8, 91.7, 69.4, 69.3, 66.2, 65.7, 65.6, 62.3, 62.2, 61.6, 54.7, 34.3, 34.2, 34.1, 32.1, 29.9, 29.8, 29.7, 29.6, 29.5, 29.4, 29.3, 29.2, 27.4, 27.3, 25.0, 24.7, 22.8, 21.4, 14.3. ³¹P-NMR (162 MHz, CDCl₃) δ −3.94, −4.01. ESI-MS (m/z): calc. for C₅₇H₁₀₉NO₁₀P 998.78. found 998.7).

### 1,2-Dioleoyl-sn-glycero-3-[1-(dodecanoyloxy)ethyl]phosphocholine chloride (2) (Also Called PP140)

Compound 2 (mixture of four diastereomers, 803 mg, 38%) was obtained as a waxy solid from DOPC and 10b in CHCl₃ (5 mL). TLC R_{f} 0.26 (CH₂Cl₂/MeOH 90:10). ¹H-NMR (400 MHz, CDCl₃) δ 6.41 (m, 1H), 5.35-5.19 (m, 5H), 4.53 (br s, 2H), 4.28-4.11 (m, 6H), 3.50 (s, 8H), 2.31-2.26 (m, 6H), 1.97 (m, 8H), 1.58-1.51 (m, 9H), 1.26-1.17 (m, 56H), 0.84 (t, ³J_{HH}=6.7 Hz, 9H). ¹³C-NMR (100 MHz, CDCl₃) δ 173.5, 173.1, 172.1, 130.2, 129.8, 91.7, 91.7, 91.6, 91.5, 69.6, 69.5, 69.4, 66.7, 66.6, 65.6, 65.5, 62.1, 62.0, 61.8, 54.7, 34.3, 34.2, 32.1, 29.9, 29.8, 29.7, 29.6, 29.5, 29.4, 29.3, 29.2, 27.4, 27.3, 25.0, 24.6, 22.8, 21.4, 14.3. ³¹P-NMR (162 MHz, CDCl₃) δ −5.6, −5.7 (2P), −5.8. ESI-MS (m/z): calc. for C₅₈H₁₁₁NO₁₀P 1012.79. found 1012.7.

### 1,2-Dioleoyl-sn-glycero-3-[(hexanoyloxy)methyl]phosphocholine chloride (3) (Also Called PP138)

Compound 3 (mixture of two diastereomers, 100 mg, 28%) was obtained as a waxy solid from DOPC and 10c in CHCl₃ (5 mL). TLC R_{f} 0.20 (CH₂Cl₂/MeOH 90:10). ¹H-NMR (400 MHz, CDCl₃) δ 5.67-5.57 (m, 2H), 5.33-5.22 (m, 5H), 4.57 (br s, 2H), 4.34-4.13 (m, 6H), 3.49 (s, 9H), 2.40-2.27 (m, 6H), 1.93 (m, 8H), 1.65-1.53 (m, 6H), 1.27-1.24 (m, 44H), 0.85 (t, ³J_{HH}=6.6 Hz, 9H). ¹³C-NMR (100 MHz, CDCl₃) δ 173.5; 173.0, 172.3, 130.2, 129.8, 83.1, 69.5, 69.4, 66.7, 66.6, 65.6, 65.3, 62.4, 62.3, 62.2, 61.6, 57.7, 34.3, 34.2, 34.0, 32.1, 31.3, 29.9, 29.7, 29.5, 29.4, 29.3, 29.2, 17.4, 27.3, 25.0, 24.3, 22.9, 22.4, 14.3, 14.0. ³¹P-NMR (162 MHz, CDCl₃) δ −3.7, −3.9. ESI-MS (m/z): calc. for C₅₁H₉₇NO₁₀P 914.69. found 914.6.

### 1,2-Dioleoyl-sn-glycero-3-[1-(hexanoyloxy)ethyl]phosphocholine chloride (4) (Also Called PP189)

Compound 4 (mixture of four diastereomers, 50.6 mg, 21%) was obtained as a waxy solid from DOPC and 10d in CHCl₃ (4 mL). TLC R_{f} 0.23 (CH₂Cl₂/MeOH 90:10). ¹H-NMR (400 MHz, CDCl₃) δ 6.43-6.37 (m, 1H), 5.34-5.27 (m, 4H), 5.21 (br s, 1H), 4.54 (br s, 2H), 4.30-4.13 (m, 6H), 3.48 (s, 9H), 2.32 (m, 4H), 1.99 (m, 8H), 1.56 (m, 9H), 1.31-1.24 (m, 44H), 0.86 (m, 9H). ¹³C-NMR (100 MHz, CDCl₃) δ 173.5, 173.1, 172.5, 172.2, 130.3, 129.9, 91.8, 69.4, 66.3, 65.8, 62.5, 61.8, 61.6, 55.0, 50.1, 34.4, 34.3, 34.2, 32.1, 31.4, 29.9, 29.7, 29.4, 29.3, 29.2, 27.4, 27.3, 25.1, 24.4, 22.9, 22.4, 21.5, 14.3, 14.1. ³¹P-NMR (162 MHz, CDCl₃) δ −5.6, −5.7, −5.8, −5.9. ESI-MS (m/z): calc. for C₅₂H₉₉NO₁₀P 928.70. found 928.7.

### 1,2-Dioleoyl-sn-glycero-3-[(propionyloxy)methyl]phosphocholine chloride (5) (Also Called PP194)

Compound 5 (mixture of two diastereomers, 103 mg, 29%) was obtained as a waxy solid from DOPC and 10e in CHCl₃ (6 mL). TLC R_{f} 0.17 (CH₂Cl₂/MeOH 90:10). ¹H-NMR (400 MHz, CDCl₃) δ 5.66-5.62 (m, 2H), 5.32-5.29 (m, 4H), 5.23 (br s, 1H), 4.60 (br s, 2H), 4.30-4.14 (m, 6H), 3.48 (s, 9H), 2.41 (q, ³J_{HH}=7.5 Hz, 2H), 2.29 (q, ³J_{HH}=7.6 Hz, 4H), 1.99-1.95 (m, 8H), 1.57-1.54 (m, 4H), 1.31-1.11 (m, 40H), 1.13 (t, ³J_{HH}=7.37 Hz, 3H), 0.83 (t, ³J_{HH}=6.9 Hz, 6H). ¹³C-NMR (100 MHz, CDCl₃) δ 173.5, 173.1, 173.0, 130.2, 129.8, 83.2, 69.5, 69.4, 69.3, 66.7, 65.6, 65.5, 62.4, 62.3, 61.8, 61.7, 54.7, 34.3, 34.2, 32.1, 29.9, 29.7, 29.5, 29.4, 29.3, 29.2, 27.4, 27.3, 25.0, 22.8, 14.3, 8.8. ³¹P-NMR (162 MHz, CDCl₃) δ −3.7, −3.8. ESI-MS (m/z): calc. for C₄₈H₉₁NO₁₀P 872.64. found 872.6.

### 1,2-Dioleoyl-sn-glycero-3-{[(dodecyloxycarbonyl)oxy]methyl}phosphocholine chloride (6) (Also Called PP91)

Compound 6 (mixture of two diastereomers, 124 mg, 31%) was obtained as a waxy solid from DOPC and 11a in CHCl₃ (6 mL). TLC R_{f} 0.23 (CH₂Cl₂/MeOH 90:10). ¹H-NMR (400 MHz, CDCl₃) δ 5.63 (m, 2H), 5.32-5.28 (m, 4H), 5.22 (br s, 1H), 4.58 (br s, 2H), 4.28-4.09 (m, 8H), 3.51 (s, 9H), 2.29 (m, 4H), 1.99-1.95 (m, 8H), 1.65-1.56 (m, 6H), 1.27-1.16 (m, 58H), 0.85 (t, ³J_{HH}=6.5 Hz, 9H). ¹³C-NMR (100 MHz, CDCl₃) δ 173.4, 173.1, 153.7, 130.2, 129.8, 86.1, 69.8, 69.4, 69.3, 66.8, 66.7, 65.6, 65.5, 62.4, 62.3, 61.6, 61.5, 54.8, 34.3, 34.2, 32.1, 29.9, 29.8, 29.7, 29.5, 29.4, 29.3, 29.2, 28.7, 27.4, 27.3, 25.8, 25.0, 22.9, 14.3. ³¹P-NMR (162 MHz, CDCl₃) δ −3.4, −3.5. ESI-MS (m/z): calc. for C₅₈H₁₁₁NO₁₁P 1028.79. found 1028.7.

### 1,2-Dioleoyl-sn-glycero-3-{1-[(dodecyloxycarbonyl)oxy]ethyl}phosphocholine chloride (7) (Also Called PP120)

Compound 7 (mixture of four diastereomers, 450 mg, 55%) was obtained as a waxy solid from DOPC and 11b in CHCl₃ (12 mL). TLC R₁ 0.28 (CH₂Cl₂/MeOH 90:10). ¹H-NMR (400 MHz, CDCl₃) δ 6.28 (m, 1H), 5.35-5.26 (m, 4H), 5.20 (br s, 1H), 4.55 (br s, 2H), 4.32-4.07 (m, 8H), 3.51 (s, 9H), 2.33-2.26 (m, 4H), 1.96 (m, 8H), 1.67-1.56 (m, 9H), 1.26-1.22 (m, 58H), 0.85 (t, ³J_{HH}=6.9 Hz, 9H). ¹³C-NMR (100 MHz, CDCl₃) δ 173.4, 173.1, 153.4, 130.2, 129.9, 95.2, 69.5, 69.4, 69.3, 66.3, 66.2, 65.7, 65.6, 65.5, 62.3, 61.7, 61.6, 61.5, 54.7, 34.3, 34.1, 32.1, 29.9, 29.8, 29.7, 29.5, 29.4, 29.3, 29.2, 28.7, 27.4, 27.3, 25.8, 25.0, 22.8, 21.5, 21.4, 14.3. ³¹P-NMR (162 MHz, CDCl₃) δ −5.6, −5.8 (2P), −5.9. ESI-MS (m/z): calc. for C₅₉H₁₁₃NO₁₁P 1042.80. found 1042.8.

### 1,2-Dioleoyl-sn-glycero-3-{1-[(dodecyloxycarbonyl)oxy]-2-methylpropyl}phosphocholine chloride (8) (Also Called PP 178)

Compound 8 (mixture of four diastereomers, 120 mg, 27%) was obtained as a waxy solid from DOPC and 11c in CHCl₃ (6 mL). TLC R_{f} 0.32 (CH₂Cl₂/MeOH 90:10). ¹H-NMR (400 MHz, CDCl₃) δ 6.05-6.01 (m, 1H), 5.38-5.27 (m, 4H), 5.24-5.20 (br s, 1H), 4.55 (br s, 2H), 4.33-4.09 (m, 8H), 3.50 (s, 9H), 2.33-2.27 (m, 4H), 1.96 (m, 8H), 1.69-1.57 (m, 6H), 1.27-1.23 (m, 58H), 0.98 (d, ³J_{HH}=6.5 Hz, 6H), 0.85 (t, ³J_{HH}=6.7 Hz, 9H). ¹³C-NMR (100 MHz, CDCl₃) δ 173.5, 173.2, 153.9, 130.3, 129.9, 100.3, 69.5, 69.4, 66.9, 65.8, 62.3, 61.8, 55.2, 55.1, 34.4, 34.2, 33.1, 32.1, 29.9, 29.8, 29.7, 29.5, 29.4, 29.3, 28.7, 27.5, 27.4, 25.8, 25.0, 22.8, 16.6, 16.0, 14.3. ³¹P-NMR (162 MHz, CDCl₃) δ −4.7, −4.8, −4.9, −5.0. ESI-MS (m/z): calc. for C₆₁H₁₁₇NO₁₁P 1070.84. found 1070.8.

### 1,2-Dioleoyl-sn-glycero-3-{4-[(dodecyloxy)-4-oxobutanoyloxy]methyl}phosphocholine chloride (9) (Also Called PP93)

Compound 9 (mixture of two diastereomers, 110 mg, 26%) was obtained as a waxy solid from DOPC and 12 in CHCl₃ (8 mL). TLC R_{f} 0.16 (CH₂Cl₂/MeOH 90:10). ¹H-NMR (400 MHz, CDCl₃) δ 5.65-5.62 (m, 2H), 5.33-5.29 (m, 4H), 5.23-5.21 (m, 1H), 4.58 (br s, 2H), 4.47 (m, 1H), 4.32-4.13 (m, 6H), 4.03 (t, ³J_{HH}=7.2 Hz, 2H), 3.49 (s, 9H), 2.68-2.60 (m, 4H), 2.31-2.27 (m, 4H), 2.00-1.95 (m, 8H), 1.61-1.56 (m, 6H), 1.27-1.23 (m, 58H), 0.85 (t, ³J_{HH}=6.9 Hz, 3H). ¹³C-NMR (100 MHz, CDCl₃) δ 173.5, 173.1, 172.5, 171.2, 130.2, 129.9, 83.2, 83.2, 69.4, 66.7, 65.5, 65.2, 62.4, 61.7, 61.6, 54.8, 34.3, 34.2, 32.1, 29.9, 29.8, 29.7, 29.5, 29.4, 29.3, 29.2, 28.9, 28.7, 28.6, 27.4, 26.1, 25.0, 22.9, 14.3. ³¹P-NMR (162 MHz, CDCl₃) δ −3.3, −3.5. ESI-MS (m/z): calc. for C₆₁H₁₁₅NO₁₂P 1084.82. found 1084.8.

3. Preparation of Liposomes

For hydrolysis rate measurements involving ³¹P-NMR spectoscopy and BET intercallation assays, liposomes were prepared by a solvent injection technique. A dry lipid film (10 μmol) was dissolved in i-PrOH (200 μL) and then injected with a syringe (flow rate: 600 μL/min) under stirring (stirring speed: 400 rpm) in the middle of the appropriate aqueous buffer medium (either Hepes 10 mM, pH 7.4 or AcOK/AcOH 10 mM, pH 4.5), at 22° C.

4. Dynamic Light Scattering Measurements

The average particle size of the liposomes and lipoplexes was measured by photon correlation spectroscopy using a Zetasizer nanoZS apparatus (Malvern Instruments, Paris, France). All measurements were conducted at 25° C. Data were analyzed using the multimodal number distribution software supplied with the instrument. For the preparation of lipoplexes, lipids were freshly solubilized in ethanol (1.24 mM) and deposited (60 μL) at the bottom of a 1.5 mL eppendorf tube. Volatiles were removed under reduced pressure to obtain a dry film. Typically, 404 μL of 60 μM solution of pCMVLuc in 4.5% glucose was added to the lipid. After 30 seconds vortexing and 15 minutes standing at room temperature, the complexes were diluted in 4.5 glucose (1096 μL) for measurements. Value for each sample is the mean of triplicate determinations (±SD).

5. DNA Retardation Assay

Freshly prepared lipoplexes at the desired N/P ratio were analyzed by electrophoresis through a 1% agarose gel. The gel was run in a 40 mM Tris-acetate-EDTA buffer, pH 8.0 (TAE). DNA migration was visualized by staining with ethidium bromide (EB, 0.5 μg/mL).

6. Ethidium Bromide Intercalation Assay

In a 96-well microplate, EB (0.28 μg) and salmon sperm DNA (2.4 μg) were first mixed in 10 mM Hepes pH 7.4 or 10 mM acetate buffer pH 4.5 (67 μL), and kept at room temperature for 2 min. Then, an ethanolic solution of cationic lipid (1.45 mM) was added to the DNA/EB mixture in order to get a N/P ratio of 3. Subsequently, fluorescence spectra were recorded for the complex solution as a function of time on a Gemini XPS Fluorescence Microplate Reader (Molecular Devices, Saint-Gregoire, France) with excitation and emission wavelengths at 546 and 600 nm, respectively. The emission intensity of the initial DNA/EB complex (I₁₀₀) and of the cationic lipid/DNA/EB solution (I₀) were taken as the corresponding references for relative intensity 100% (F₁₀₀) and 0% (F₀), respectively. Further, the emission of the DNA/EB complex at 600 nm (I) was measured and the fluorescence percentage was calculated according to the following equation: F(%)=((I−I₀)/(I₁₀₀−I₀))×100.

7. Cell Culture

All cells were maintained at 37° C. in a 5% CO₂ humid chamber. BHK-21 cells were grown in DMEM-F12 medium containing FBS (10%), penicillin (100 units/mL), and streptomycin (100 μg/mL). A549 and Calu-3 cells were grown in DMEM-F12 medium containing FBS (10%), penicillin (100 units/mL), streptomycin (100 μg/mL), and Hepes (5 mM). NCI-H292 cells were grown in RPMI 1640 supplemented with FBS (10%), sodium pyruvate (1 mM), L-glutamine (2 mM), penicillin (100 units/mL), and streptomycin (100 μg/mL). U87Luc cells were grown in DMEM supplemented with FBS (10%), penicillin (100 units/mL), streptomycin (100 μg/mL), and L-glutamine (2 mM). Selection of U87Luc and A549Luc cells was performed by adding G418 (0.8 mg/mL) to the cell culture medium. At confluence, cells were released with trypsin (0.5% in PBS), centrifuged (4° C., 5 min, 120 g) counted and transferred into a 96-well plate (Becton-Dickinson) in 100 μL culture medium at a density of 6,000 cells/well for DNA delivery experiments, and at a density of 8,000 cells/well for siRNA delivery experiments. Plates were maintained at 37° C. in a 5% CO₂ humid chamber for 24 h before each experiment.

8. Lactate Dehydrogenase Assay

Lactate dehydrogenase (LDH) release was used to assess the cytotoxicity of the formulations used in the transfection experiments described as outlined. Typically, at the end of each transfection experiment, the culture medium was removed and placed in another 96-wells plate. LDH activity was measured using a commercial kit (Cytotoxicity Detection Kit Plus, Roche Applied Science) according to the manufacturer's instructions. LDH activity was expressed as the percentage of the maximal LDH release obtained after treatment of the cells with the lysis solution kit (5 μL). Less than 10% LDH release was regarded as a measure of non-significant toxicity. Value for each sample is the mean of triplicate determinations (±SD).

9. DNA Transfection Experiments

Lipids were freshly solubilized in ethanol at 1.24 mM concentration. Typically, 40.40 μL of 60 μM solution of DNA (pCMVLuc [56]) in 4.5% glucose was added to the lipid (either 2.00, 5.85 or 9.75 μL deposited at the bottom of a 500 μL eppendorf tube and dried under vacuum). After gentle vortex agitation (30 sec), the complexes were allowed to stand at room temperature for 30 min before addition (10 μL, i.e. 0.2 μg DNA) into each well (triplicate). Cells were then let to grow in the incubator without further handling. Luciferase gene expression was assessed 24 h later using a commercial kit according to the manufacturer's protocol (Promega, Charbonnières, France). Cells were washed with PBS (100 μL) and lyzed with the Promega lysis buffer (20 μL). After agitation for 15 min, PBS (150 μL) was added. A 5 μL aliquot was transferred to a white microplate and luminescence was recorded for 1 sec with a luminometer (Berthold Centro LB960 XS, Thoiry, France) upon addition of the luciferin substrate (35 μL). Value for each sample is the mean of triplicate determinations (±SD).

10. siRNA Delivery Experiments

The luciferase gene silencing experiments were performed with a RNA duplex (siLuc) of the sense sequence: 5′-CUU ACG CUG AGU ACU UCG A. Untargeted RNA duplex (sic) was of sequence: 5′-CGU ACG CGG AAU ACU UCG A. Lipids were freshly solubilized in ethanol at 0.5 mM concentration. Typically, 40 μL of 100 nM solution of siRNA (either siLuc or sic) in 4.5% glucose was added to the lipid (either 8, 16 or 32 μL deposited at the bottom of a 500 μL eppendorf tube and dried under vacuum). After vortex agitation for 30 sec, the complexes (10 μL, i.e. 1 pmol) were added into each well (triplicate). Cells were then let to grow in the incubator without further handling. Luciferase gene expression was assessed 48 h later as described above, except PBS volume added was different (200 μL). The transfection efficiency was expressed as the residual luciferase activity when compared to non-treated cells (100%). Value for each sample is the mean of triplicate determinations (±SD).

### Results

### Example 1

### Hydrolytic Properties of Compounds (1)-(9)

The hydrolytic stability under neutral and acidic conditions in a model experiment involving ³¹P-NMR measurements were assessed for compounds 1-9. Phosphotriesters 1-9 and phosphodiester DOPC display ³¹P chemical shifts differing by 5-6 ppm in aqueous media allowing a precise monitoring of the hydrolysis reaction. The cationic lipids were formulated into liposomes using an injection technique as described hereabove. Liposomes were prepared at pH 7.4 and pH 4.5, and incubated at 37° C. Periodical acquisition of ³¹P-NMR spectra allowed determination of the time t, required for 50% hydrolysis (Table 3).

TABLE 3
Hydrolytic stability of amphiphiles 1-9. Compounds formulated into
liposomes were incubated at 37° C. and hydrolysis was monitored
by ³¹P-NMR measurements. Time required for 50% hydrolysis (t_{1/2} )
was calculated from the theoretical curve fitting with the experimental data.

| | | Compound | | | | | | | | | |
|---|---|---|---|---|---|---|---|---|---|---|---|
| | | 1 | 2 | 3 | 4 | 5 | 6 | 7 | 8 | 9 | EDOPC |
| t_{1/2} (h) | pH 7.4 | 173 | 10 | 280 | 5.4 | 320 | 385 | 29 | 497 | 319 | —^{a} |
| | pH 4.5 | 94 | 2.1 | 179 | 1.3 | 170 | 108 | 27 | 498 | 150 | —^{a} |

^{a}See in the body text.

Cationic conjugates (1-5, 6 and 9) display lowered stability from neutral to acidic pH. Surprisingly, this was not observed in the case of carbonates 7 and 8 as these two compounds are hydrolyzed at the same rate whatever the pH value. This reveals that the results obtained in this experiment cannot be analyzed only taking into account the chemical reactivity of the acetal moiety but should rather be interpreted with regard to the respective sensitivity of the neighboring carbonate and ester moieties towards hydrolysis. Indeed hydrolysis rate depends on the reaction mechanism (specific base catalyzed, water catalyzed or specific acid catalyzed) but also on steric and electronic effects at the acetal center. Throughout the whole compounds series, the introduction of a methyl substituent on the acetal linker invariably provokes an increase in the hydrolysis rate, both under acidic and physiological pH (2, 4, and 7 vs 1, 3, and 6, respectively). At the opposite, data obtained for derivative 8 indicate that the introduction of an iso-propyl substituent at the ketal moiety strongly decreases the hydrolysis rate. This observation may be explained either by a more hydrophobic environment established in the acetal region or some steric hindrance, which may hamper the approach of the ionic species (H₃O⁺ or OH⁻) or water molecule that is required for the hydrolysis to occur, whatever the mechanism involved. Finally, in liposome preparations based on EDOPC no trace of hydrolysis could be detected over a 10 days period.

### Example 2

### Lipoplexes Formation and Characterization

The ability of cationic lipids 1-9 to interact electrostatically with nucleic acid to form lipoplexes was assessed by conventional electrophoretic DNA retardation assay in which a full retard of plasmid DNA is observed at a charge ratio (lipid amine/DNA phosphate ratio, N/P) corresponding to electroneutrality. Data indicate that the introduction of a hydrophobic substituent onto the phosphate group of DOPC, whatever its length (C₂ to C₁₂), has no significant effect on the ability of the ammonium headgroup of the molecule to interact electrostatically with the DNA polyphosphate backbone. All the compounds investigated herein fully retarded DNA at N/P between 1 and 1.2. The hydrodynamic diameter of pCMVLuc complexes with cationic vectors 1-9 was also determined by dynamic laser light scattering experiments. In all the cases, we observed a unique population of complexes with a narrow distribution and diameter in the range 315-820 nm (Table 4). In comparison, lipoplexes prepared from EDOPC revealed slightly larger (902 nm). Surprisingly, the 3 compounds incorporating a non-substituted acetal bridge (1, 6, and 9) led to the formation of negatively charged lipoplexes at a charge ratio N/P=3 (−26.0, −26.4, and −18.0 mV, resp.) whereas all the other cationic lipids investigated herein led to positively charged particles (+43.5 to +62.9 mV).

TABLE 4
Particle size and zeta potential (ζ) of lipoplexes obtained
from lipids 1-9 and EDOPC, and pCMVLuc as measured by dynamic
light scattering. Lipoplexes were freshly prepared in 5%
glucose, at charge ratio N/P 3 and 25° C. Data reported
are the mean of three independent measurements ± SD.

| Compound | Particle size (nm) | Zeta potential (mV) |
|---|---|---|
| 1 | 535.3 ± 52.9 | −26.0 |
| 2 | 669.8 ± 17.7 | +46.4 |
| 3 | 656.7 ± 42.1 | +55.4 |
| 4 | 484.5 ± 40.6 | +49.0 |
| 5 | 464.8 ± 56.6 | +51.1 |
| 6 | 315.4 ± 46.4 | −26.4 |
| 7 | 819.3 ± 79.8 | +62.9 |
| 8 | 721.8 ± 65.7 | +48.5 |
| 9 | 364.3 ± 34.6 | −18.0 |
| EDOPC | 901.7 ± 18.8 | +43.5 |

### Example 3

### Plasmid DNA Transfection in BHK-21 Cell Line and Cell Viability

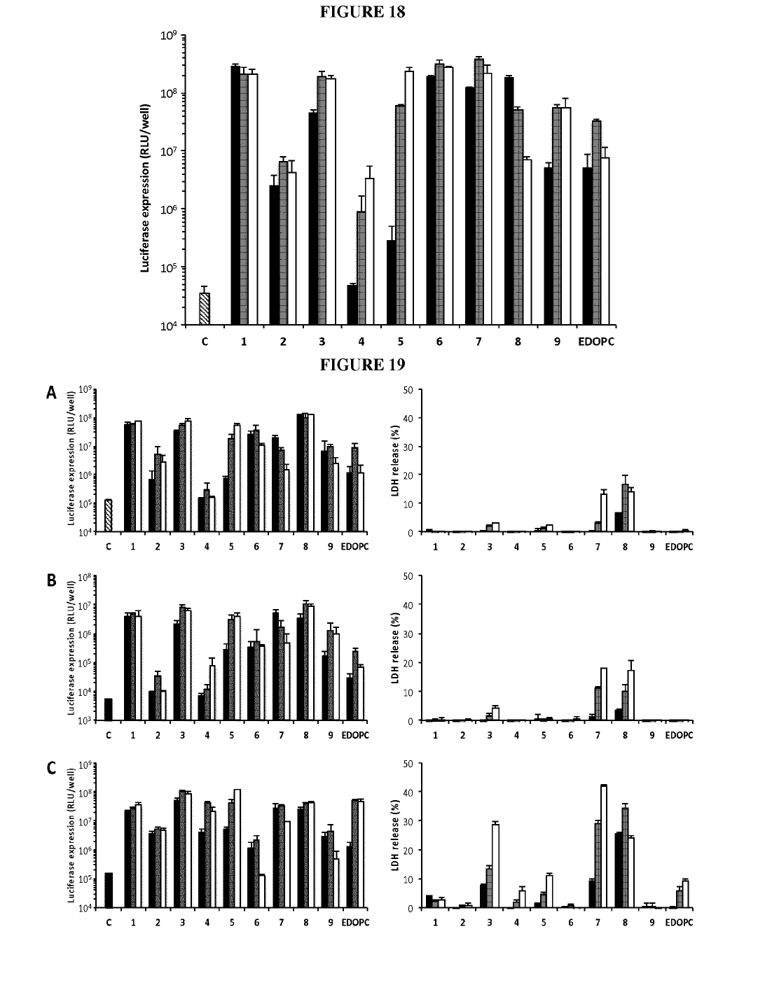

The biological transfection activity of the compounds (1-9) was firstly assessed using the BHK-21 cell line, and pCMVLuc, a plasmid DNA encoding for the firefly luciferase gene under the control of a strong promoter. The levels of luciferase expression induced by each lipoplex in BHK-21 are shown in FIG. 18. Compounds 2 and 4 that displayed higher sensitivity to hydrolysis were least efficient to transfect cells with pDNA. All the other lipid carriers investigated herein showed a high transfection efficiency. Notably compounds 1, 3, 5, 6, 7 and 8 were more effective than control phospholipid EDOPC. Notably, compound 5 which only differs from EDOPC by the presence of a functionalized acetal connector is more effective than EDOPC for a N/P ratio of 3 and 5. This illustrates the impact of the presence of the hydrolyzable connector on the transfection activity of the compounds. However, there is no very clear relationship between transfection efficiency and the previously determined hydrolytic stability of the compounds.

The influence of the helper lipid DOPE (1,2-dioleoyl-sn-glycero-3-phosphoethanolamine) on the transfection efficiency of compounds 1 to 9 (1-9/DOPE mixtures, 1:1 molar ratio) to deliver pDNA to BHK-21 cells was assessed. Surprisingly, when associated with DOPE, the transfection efficiency of compounds 1-9 was reduced by up to four orders of magnitude. Cationic phospholipid 9 was unable to assist DNA transfection when mixed with DOPE. On the contrary, DOPE improved the transfection property of the control compound EDOPC by a fifteen-fold factor.

The impact of DNA transfection on cell viability was then assessed by a LDH release assay. Cell death by necrosis is systematically accompanied by damage to cell plasma membrane and subsequent release of cytoplasmic material into the supernatant. Thus, loss of cell viability can be directly monitored by measuring the activity of lactate dehydrogenase (LDH), a cytosolic enzyme, in the extracellular compartment. The lipoplex formulations obtained from compounds 1-7 and 9 did not significantly perturb the membrane integrity of the cells since the LDH release did not exceed 5-6%. Lipoplex based on lipid 8 provoked a DLH release of 11% LDH, which may be considered as indicative of low cell cytotoxicity. A general trend is that lipids in the carbonate series (compounds 6, 7, and 8) are a little bit more aggressive towards the BHK-21 cell line than lipids in the ester series (compounds 1-5 and 9) in which the LDH release is at most 3% for all the tested N/P ratio (1, 3 and 5).

Compound 1 (namely PP94)—which comprises a C₁₁ alkyl chain—exhibits the most favorable ratio between pDNA transfection efficiency and cytotoxicity.

### Example 4

### Plasmid DNA Transfection in Pulmonary Cell Lines and Cell Viability

Lung disorders are among the most representative causes of mortality and morbidity according to the World Health Organization (WHO), and identifying powerful and cost-effective treatments, in particular gene therapy, is a matter of high priority. Depending on the respiratory disease and the therapeutic goal, the target cells in the lung can vary from epithelial cells, alveolar cells, macrophages, respiratory stem cells or endothelial cells. All these cell types, except the latter two, can be directly accessed via inhalation or instillation of nucleic acid containing nanoparticles. Local pulmonary gene delivery may be especially advantageous as it reduces systemic side effects and do not lead to en-route interactions with serum proteins. As a significant consequence, the required dose of nucleic acid can be substantially reduced.

The ability of compounds 1-9 to transfect plasmid DNA was assessed for the following cell lines: A549 cells (alveolar, human lung carcinoma), Calu-3 cells (bronchial, epithelial lung adenocarcinoma), and NCI-H292 cells (bronchial, human lung mucoepidermoid carcinoma). The results concerning transfection activity and LDH release for compounds 1 to 9 are shown in FIGS. 19A, 19B and 19C for cell line A549, Calu-3 and NCI-H292, respectively.

As previously observed on the BHK-21 cell line, compounds 2 and 4 display a poor transfection activity in A549 and Calu-3 cells but provide intermediate efficacy on the NCI-H292 cell line. Notably, compounds 1, 3, and 8 systematically led to the highest cell transfection levels, whatever the cell line considered. In addition, transfection appears only poorly sensitive to the charge ratio with these compounds and maximum efficacy most of the time was achieved at NIP 1. At the opposite, transfection efficiency of compound 5 was improved from N/P 1 to N/P 5 by one to two orders of magnitude. Compounds 6, 7, and 9 exhibit significant transfection activity. One may conclude that A549, Calu-3, and NCI-H292 cell line may be efficiently transfected with lipoplexes prepared with most of the tested compounds. Noteworthy, all the compounds (except compound 6) display significant transfection activity in NCI-H292 cell line which seems to show that the mucus secreted by said cell line is not necessarily a critical barrier preventing transfection in respect to compounds 1-5 and 7-9.

Compound 1 which comprises a C₁₁ alkyl chain seems to be as effective as compound 3 which comprises a C₅ alkyl chain. On the contrary, compound 5 which comprises a short alkyl chain (C₂) should be used at higher charge ratio in order to provide the same efficiency than compounds 1 and 3. This illustrates that compound 5 is less effective than compounds 1 and 3 to transfect pDNA and thus shows the impact of the length of the alkyl chain (namely R⁸) on the activity of the compounds of the invention.

As observed previously, the introduction of a methyl group on the ketal bridge in the ester series (1, and 3 vs. 2, and 4, respectively) is detrimental to the transfection efficiency of the compounds. On the contrary, in the carbonate series, increasing steric hindrance or hydrophobicity at this position (H, Me, i-Pr for 6 to 8, resp.) revealed rather beneficial and improved transfection efficiency by up to two orders of magnitude in some cases.

In term of cytotoxicity, carbonates 7 and 8 that regularly stand as good transfection agents provoke significant LDH release in the cell lines investigated. This effect is especially important with NCI-H292 cells for which up to 30-40% LDH release was observed. Compounds 1 and 3 that display similar transfection efficiency are devoid of significant cytotoxicity (LDH<5%), at least in the A549 and Calu-3 cell lines. Compound 1 is particularly interesting since it display very low cell toxicity as shown by LDH release assay and display DNA transfection activity in all tested cell lines.

### Example 5

### In Vitro Delivery of siRNA

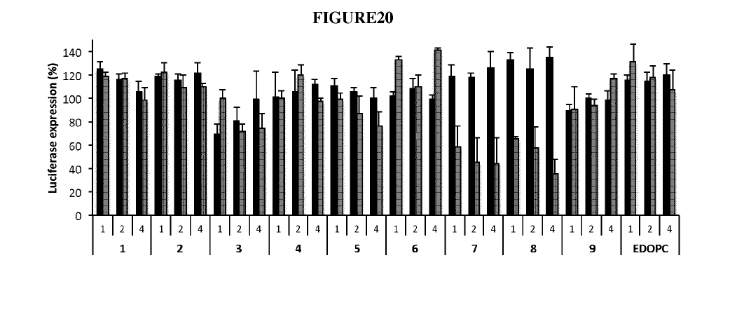

The ability of compounds 1-9 to deliver small interfering RNA was assessed in the U87 epithelial-like cell line (human glioblastoma-astrocytoma) that has been stably transformed to express a luciferase gene (U87Luc cells). The delivery of a specific siRNA (siLuc) into the cytosol of these cells should induce reduction of luciferase expression, when compared to a mismatched siRNA (sic) used as a negative control. The siRNA lipoplexes were added to the cells in culture medium and luciferase activity was measured after a 48 h incubation period. Knockdown of the luciferase expression in the U87Luc cells as mediated by the nine deciduous-charge cationic lipids is shown in FIG. 20.

Carbonate derivatives 7 and 8 were able to reduce luciferase expression down to 40-30%, in a dose-dependent manner. The other compounds, namely 1-6 and 9, were poorly effective to induce a reduction of luciferase expression whereas they were particularly efficient to promote plasmid DNA transfection. Such results showed that a good transfection activity for DNA is not predictive of good transfection activity for RNA and vice versa.

### Part C

### Optimization

Various compounds were prepared in order to determine the impact of the length of PEG spacers and/or the nature of the hydrolyzable connectors.

### Materials and Methods

Unless otherwise stated, all chemical reagents were purchased from Alfa Aesar (Bischeim, France) and used without purification. 1,2-Dioleoyl-sn-glycero-3-phosphocholine (DOPC), 1,2-dipalmitoyl-sn-glycero-3-phosphocholine (DPPC) and Triton X-100® were from Sigma-Aldrich (Saint-Quentin Fallavier, France). When required, solvents were dried by standard procedures just before use. Thin layer chromatography (TLC) was performed on precoated plates (0.25 mm Silica Gel 60, F254, Merck, Darmstadt, Germany). Products were purified by chromatography over silica gel (Silica Gel 60, 40-63 μm, Merck, Darmstadt, Germany). NMR spectra were recorded on Bruker 300 MHz Avance DPX and 400 MHz Avance III instruments. ¹H-, ¹³C-, and ³¹P-NMR chemical shifts δ are reported in ppm relative to their standard reference (¹H: CHCl₃ at 7.27 ppm, HDO at 4.63 ppm, CD₂HOD at 3.31 ppm; ¹³C: CDCl₃ at 77.0 ppm, CD₃OD at 49.0 ppm; ³¹P: H₃PO₄ at 0.00 ppm). IR spectra were recorded on a FT-IR Nicolet 380 spectrometer in the ATR mode and absorptions values ν are in wave numbers (cm⁻¹). Mass Spectra (MS) were recorded on an Agilent technology 6520 Accurate Mass QToF, using electrospray ionization (ESI) mode. Mass data are reported in mass units (m/z).

Series 1: conjugates wherein R⁸ is

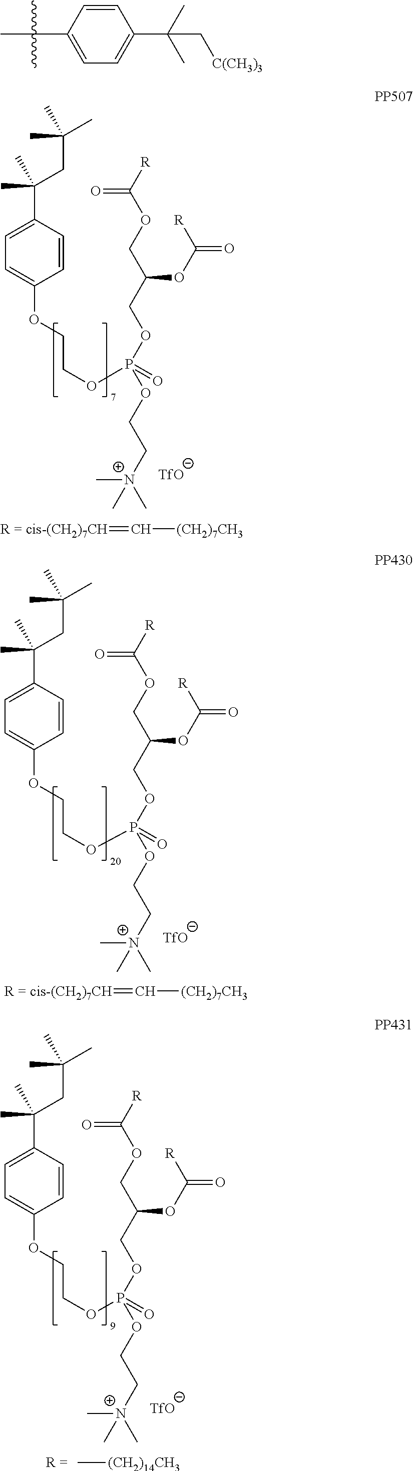

### Series 2: Conjugates Wherein R⁸ is an Alkyl Chain and R₁═R₂=Cis (CH₂)₇CH═CH(CH₂)₇CH₃

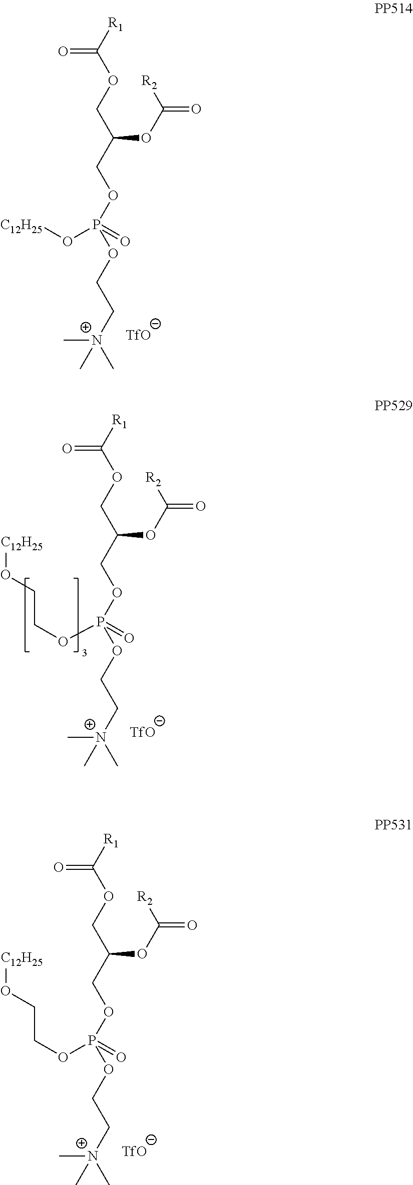

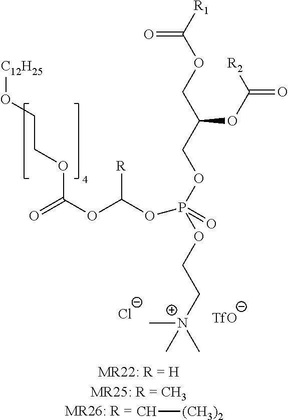

It should be noticed that PP514 is a control compound which does not belong to the instant invention.

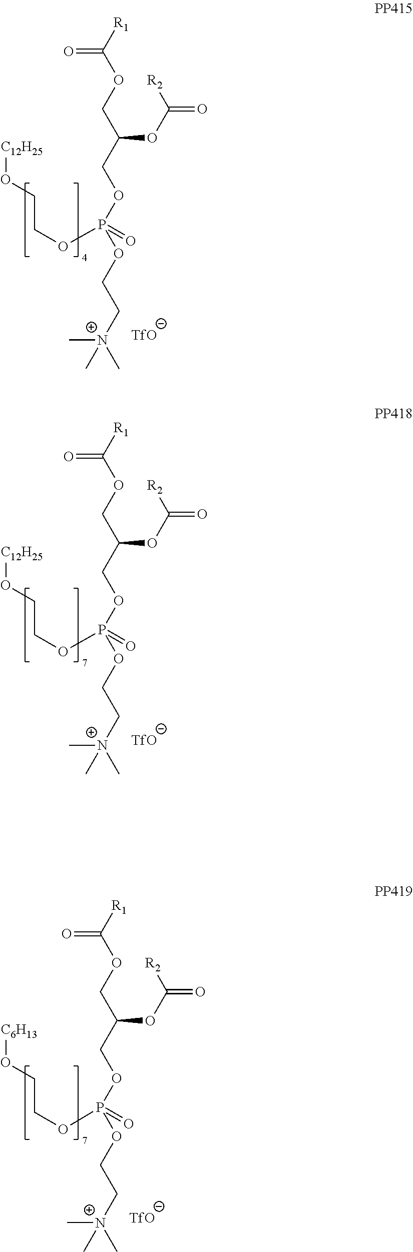

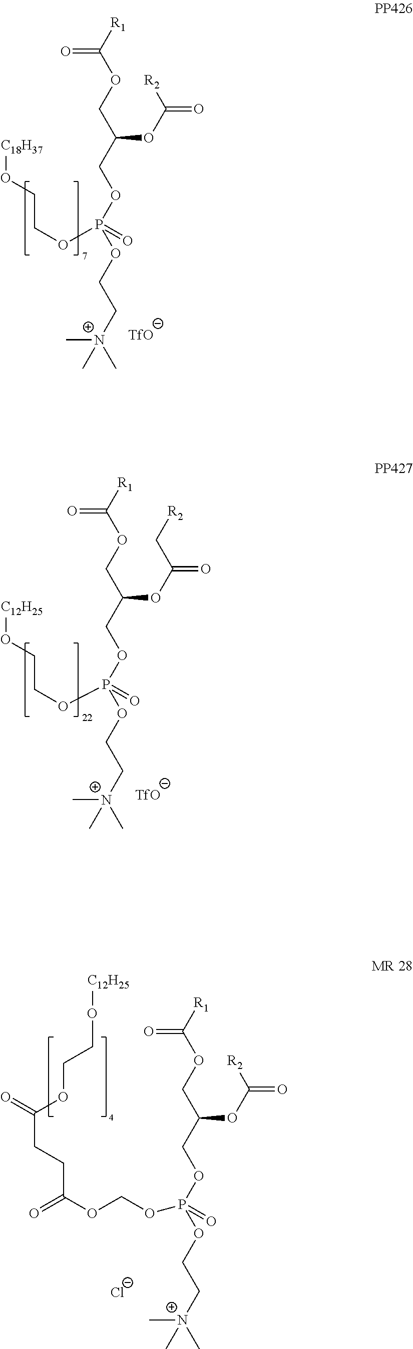

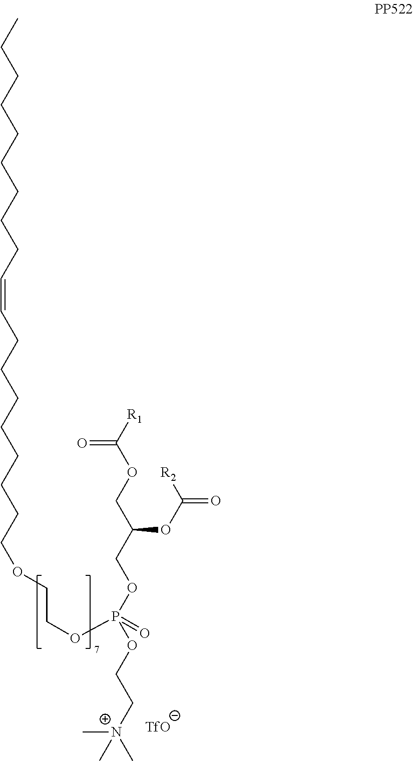

### 1. Synthesis

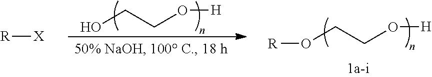

| Name | R | X | n | Yield (%) |
|---|---|---|---|---|
| 1a | C₆H₁₃ | Cl | 9 | 78 |
| 1b^{(a)} | C₁₂H₂₅ | — | 0 | — |
| 1c | C₁₂H₂₅ | Br | 1 | 79 |
| 1d | C₁₂H₂₅ | Br | 3 | 50 |
| 1e | C₁₂H₂₅ | Br | 4 | 33 |
| 1f | C₁₂H₂₅ | Br | 9 | 73 |
| 1g | C₁₂H₂₅ | Br | 22 | 27 |
| 1h | C₁₈H₃₇ | Cl | 9 | 60 |
| 1i | C₁₈H₃₅ (oleyl)^{(b)} | Br | 9 | 47 |

^{(a)}Commercially available.
^{(b)}Oleyl bromide was prepared as reported in the literature (Basabe, P et al. Eur J Med Chem 2010; 45: 4258-4269)

### Synthesis of Compounds 1a-i.

To a solution of oligoethyleneglycol (5 eq.) was added 50% NaOH (5 eq.). The mixture was refluxed at 100° C. for 30 minutes until the color changed to brown. Then, the corresponding electrophile (1 eq.) was added and the resulting mixture was heated for additional 18 h. The reaction mixture was cooled down and diluted with water. In a typical extraction process, the product was extracted twice with AcOEt. Combined organic layers were then washed five times with water in order to remove unreacted starting polyethyleneglycol. Then, organic layer was dried over Na₂SO₄, filtered and concentrated under reduced pressure affording a brownish residue. Pure compounds have been isolated after a flash silica gel column chromatography using CH₂Cl₂/MeOH (98/2 to 90/10) as eluent.

### Compound 1a

Recovered as a yellow oil (4.15 g, 8.57 mmoles). TLC Rf 0.44 (CH₂Cl₂/MeOH 95:5). ¹H-NMR (300 MHz, CDCl₃) δ 3.71-3.54 (m, 36H), 3.42 (t, ³J_{HH}=6.7 Hz, 2H), 1.55 (t, ³J_{HH}=7.1 Hz, 2H), 1.29-1.26 (m, 6H), 0.86 (t, ³J_{HH}=6.5 Hz, 3H). ¹³C-NMR (100 MHz, CDCl₃) δ 72.7, 71.7, 70.7, 70.5, 70.2, 61.9, 31.8, 29.7, 25.9, 22.7, 14.2. FT-IR (thin film) ν 3478, 2861, 1455, 1349, 1296, 1248, 1097, 945.

### Compound 1c.

Recovered as a yellow oil (97 mg, 0.45 mmoles). TLC Rf 0.15 (EP/Et₂O 80:20). ¹H-NMR (400 MHz, CDCl₃) δ 3.70 (t, ³J_{HH}=4.2 Hz, 2H), 3.53 (t, ³J_{HH}=4.4 Hz, 2H), 3.45 (t, ³J_{HH}=6.7 Hz, 2H), 1.86 (br, 1H), 1.58-1.54 (m, 2H), 1.40-1.24 (m, 18H), 0.86 (t, ³J_{HH}=6.7 Hz, 3H). ¹³C-NMR (100 MHz, CDCl₃) δ 71.9, 71.6, 62.1, 32.1, 29.9, 29.8, 29.7, 29.6, 26.3, 22.9, 14.3. FT-IR (thin film) ν 3458, 2921, 2852, 1465, 1361, 1120, 1064.

### Compound 1d.

Recovered as a yellow oil (150 mg, 0.49 mmoles). TLC Rf 0.41 (CH₂Cl₂/MeOH 96:4). ¹H-NMR (300 MHz, CDCl₃) δ 3.69-3.53 (m, 12H), 3.41 (t, ³J_{HH}=6.8 Hz, 2H), 2.64 (br, 1H), 1.55-1.51 (m, 2H), 1.29-1.21 (m, 18H), 0.84 (t, ³J_{HH}=6.9 Hz, 3H). FT-IR (thin film) ν 2953, 2929, 2914, 2848, 1493, 1470, 1307, 1149, 1136, 958.

### Compound 1e.

Recovered as a yellow oil (2.63 g, 7.14 mmoles). TLC Rf 0.68 (CH₂Cl₂/MeOH 95:5). ¹H-NMR (300 MHz, CDCl₃) δ 3.70-3.54 (m, 18H), 3.41 (t, ³J_{HH}=7 Hz, 2H), 1.54 (t, ³J_{HH}=7 Hz, 2H), 1.28-1.22 (m, 18H), 0.84 (t, ³J_{HH}=6.8 Hz, 3H). ¹³C-NMR (100 MHz, CDCl₃) δ 72.8, 72.2, 71.7, 70.7, 70.6, 70.5, 70.2, 61.9, 32.1, 29.8, 29.6, 29.5, 26.3, 22.8, 14.3. FT-IR (thin film) ν 3485, 2921, 2852, 1738, 1458, 1245, 1098, 968.

### Compound 1f

Recovered as a yellow oil (4.56 g, 8.02 mmoles). TLC Rf 0.6 (CH₂Cl₂/MeOH 95:5). ¹H-NMR (300 MHz, CDCl₃) δ 3.71-3.54 (m, 36H), 3.42 (t, ³J_{HH}=6.8 Hz, 2H), 1.55 (t, ³J_{HH}=7 Hz, 2H), 1.25-1.23 (m, 18H), 0.86 (t, ³J_{HH}=6.5 Hz, 3H). ¹³C-NMR (100 MHz, CDCl₃) δ 72.7, 71.7, 70.8, 70.5, 70.2, 61.9, 32.1, 29.8, 29.7, 29.5, 26.3, 22.8, 14.3. FT-IR (thin film) ν 3480, 2921, 2853, 1455, 1349, 1296, 1249, 1101, 946, 913.

### Compound 1g.

Recovered as a yellow oil which solidified upon standing at room temperature (3.2 g, 2.74 mmoles). TLC Rf 0.36 (CH₂Cl₂/MeOH 95:5). ¹H-NMR (300 MHz, CDCl₃) δ 3.70-3.53 (m, 90H), 3.40 (t, ³J_{HH}=6.9 Hz, 2H), 1.53 (t, ³J_{HH}=6.9 Hz, 2H), 1.29-1.16 (m, 18H), 0.84 (t, ³J_{HH}=6.6 Hz, 3H). ¹³C-NMR (100 MHz, CDCl₃) δ 72.7, 71.7, 70.7, 70.5, 70.2, 61.9, 32.1, 29.8, 29.7, 29.5, 26.3, 22.8, 14.3. FT-IR (thin film) ν 3504, 2857, 1456, 1348, 1248, 1097, 947, 845.

### Compound 1h.

Recovered as a yellow oil (4.30 g, 6.59 mmoles). TLC Rf 0.5 (CH₂Cl₂/MeOH 95:5). ¹H-NMR (300 MHz, CDCl₃) δ 3.64-3.60 (m, 36H), 3.42 (t, ³J_{HH}=6.8 Hz, 2H), 1.55 (t, ³J_{HH}=7 Hz, 2H), 1.27-1.23 (m, 30H), 0.85 (t, ³J_{HH}=6.5 Hz, 3H). ¹³C-NMR (100 MHz, CDCl₃) δ 72.7, 71.7, 70.7, 70.5, 70.2, 61.9, 32.1, 29.8, 29.7, 29.5, 26.3, 22.8, 14.3. FT-IR (thin film) ν 2916, 2849, 1467, 1113, 913, 743.

### Compound 1i.

Recovered as a clear oil (310 mg, 0.46 mmoles). TLC Rf 0.35 (CH₂Cl₂/MeOH 95:5). ¹H-NMR (400 MHz, CDCl₃) δ 5.32 (m, 2H), 3.71-3.55 (m, 32H), 3.42 (t, ³J_{HH}=6.8 Hz, 2H), 1.99-1.98 (m, 4H), 1.56-1.53 (m, 2H), 1.26-1.23 (m, 22H), 0.86 (t, ³J_{HH}=6.7 Hz, 3H). ¹³C-NMR (100 MHz, CDCl₃) δ 129.9, 129.8, 72.6, 71.6, 70.6, 70.5, 70.3, 70.1, 61.8, 31.9, 29.8, 29.7, 29.6, 29.5, 29.4, 29.3, 29.2, 27.2, 26.1, 22.7, 14.1. FT-IR (thin film) ν 2921, 2853, 1219, 1106, 913, 772, 744.

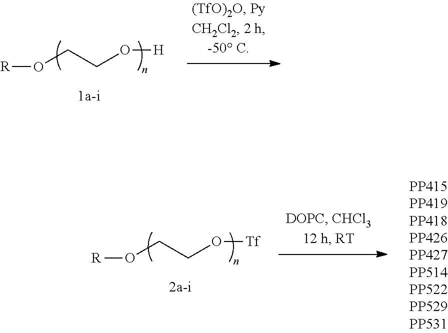

### General Procedure for the Preparation of Trifluoromethanesulfonyl Esters 2a-i.

Triflic anhydride (1.2 eq.) was added dropwise to a solution of 1a-i (1 eq.) and pyridine (1.2 eq.) in freshly distilled CH₂Cl₂, at −50° C. The reaction mixture was stirred under argon at −50° C. for 2 h before it was poured into ice-cold water. The organic layer was recovered and washed twice with cold water. The organic layer was dried over anhydrous MgSO₄, filtered and concentrated under reduced pressure at 20° C. The corresponding trifluoromethanesulfonyl esters 2a-i were obtained and used in the next step without further purification. Due to relative instability of the products, ¹H NMR spectrum was recorded immediately after isolation.

### Compound 2a.

Recovered as a yellow oil in 92% yield (580 mg, 0.92 mmoles). ¹H-NMR (300 MHz, CDCl₃) δ 4.61 (t, ³J_{HH}=4.6 Hz, 2H), 3.81 (t, ³J_{HH}=4.3 Hz, 2H), 3.67-3.56 (m, 32H), 3.43 (t, ³J_{HH}=6.7 Hz, 2H), 1.54 (t, ³J_{HH}=6.0 Hz, 2H), 1.26 (m, 6H), 0.85 (t, ³J_{HH}=5.8 Hz, 3H).

### Compound 2b.

Recovered as a clear oil in 88% yield (280 mg, 0.88 mmoles). ¹H-NMR (300 MHz, CDCl₃) δ 4.52 (t, ³J_{HH}=6.53 Hz, 2H), 1.83-1.78 (m, 2H), 1.42-1.24 (m, 20H), 0.86 (t, ³J_{HH}=5 Hz, 3H).

### Compound 2c.

Recovered as a clear oil in 92% yield (130 mg, 0.36 mmoles). ¹H-NMR (300 MHz, CDCl₃) δ 4.59 (t, ³J_{HH}=4.4 Hz, 2H), 3.71 (t, ³J_{HH}=4.5 Hz, 2H), 3.46 (t, ³J_{HH}=6.6 Hz, 2H), 1.56 (m, 2H), 1.24 (m, 20H), 0.86 (t, ³J_{HH}=6.3 Hz, 3H).

### Compound 2d.

Recovered as a clear oil in 99% yield (58 mg, 0.13 mmoles).

### Compound 2e.

Recovered as a yellow oil in 78% yield (385 mg, 0.78 mmoles). ¹H-NMR (300 MHz, CDCl₃) δ 4.62 (t, ³J_{HH}=4.6 Hz, 2H), 3.82 (t, ³J_{HH}=4.3 Hz, 2H), 3.68-3.55 (m, 14H), 3.42 (m, 2H), 1.55 (m, 2H), 1.23 (m, 18H), 0.85 (t, ³J_{HH}=6.4 Hz, 3H).

### Compound 2f.

Recovered as a yellow oil in 82% yield (655 mg, 0.82 mmoles). ¹H-NMR (300 MHz, CDCl₃) δ 4.62 (t, ³J_{HH}=3.8 Hz, 2H), 3.81 (t, ³J_{HH}=4.2 Hz, 2H), 3.67-3.56 (m, 32H), 3.43 (t, ³J_{HH}=6.7 Hz, 2H), 1.53 (t, ³J_{HH}=6.1 Hz, 2H), 1.23 (m, 18H), 0.85 (t, ³J_{HH}=6.3 Hz, 3H).

### Compound 2g.

Recovered as a yellow oil in 82% yield (1.19 g, 0.90 mmoles). ¹H-NMR (300 MHz, CDCl₃) δ 4.61 (t, ³J_{HH}=4.1 Hz, 2H), 3.80 (t, ³J_{HH}=4.2 Hz, 2H), 3.67-3.54 (m, 86H), 3.41 (t, ³J_{HH}=6.9 Hz, 2H), 1.54 (t, ³J_{HH}=6.7 Hz, 2H), 1.22 (m, 18H), 0.85 (t, ³J_{HH}=6.1 Hz, 3H).

### Compound 2h.

Recovered as a yellow oil in 82% yield (585 mg, 0.82 mmoles). ¹H-NMR (300 MHz, CDCl₃) δ 4.60 (t, ³J_{HH}=3.7 Hz, 2H), 3.82 (t, ³J_{HH}=4.1 Hz, 2H), 3.66-3.59 (m, 32H), 3.45 (t, ³J_{HH}=6.9 Hz, 2H), 1.55 (t, ³J_{HH}=6.0 Hz, 2H), 1.22 (m, 30H), 0.86 (t, ³J_{HH}=5.9 Hz, 3H).

### Compound 2i.

Recovered as a yellow oil in 99% yield (120 mg, 0.15 mmoles). ¹H-NMR (300 MHz, CDCl₃) δ 5.37-5.30 (m, 2H), 4.64-4.62 (m, 2H), 3.85-3.82 (m, 2H), 3.68-3.55 (m, 32H), 3.42 (t, ³J_{HH}=6.5 Hz, 2H), 1.99-1.95 (m, 4H), 1.62-1.58 (m, 2H), 1.32-1.24 (m, 22H), 0.86 (t, ³J_{HH}=6.7 Hz, 3H).

### Preparation of the Cationic Lipids.

To a solution of DOPC in dry CHCl₃ was added a solution of the corresponding freshly prepared trifluoromethanesulfonyl ester 2a-i (2.60 eq.) in dry CHCl₃. The resulting reaction mixture was stirred at room temperature for 20 h under argon. The reaction mixture was then concentrated under reduced pressure at room temperature. Pure products were obtained after purification by flash chromatography over silica gel (CH₂Cl₂/MeOH: 98/2 to 90/10).

### Compound PP419.

This compound was prepared from DOPC (275 mg, 0.35 mmol) and 2a (580 mg, 0.92 mmol) in dry CHCl₃ (10 mL). Column chromatography yielded the expected compound PP419 in 55% yield (271 mg, 0.190 mmol) as a clear oil (mixture of 2 diastereomers). TLC R_{f} 0.35 (CH₂Cl₂/MeOH 90:10). ¹H-NMR (400 MHz, CDCl₃) δ 5.32-5.29 (m, 4H), 5.23-5.20 (br s, 1H), 4.53 (br s, 2H), 4.31-4.10 (m, 6H), 3.85 (m, 2H), 3.69-3.52 (m, 26H), 3.41 (t, ³J_{HH}=6.8 Hz, 2H), 3.28 (s, 9H), 2.29 (q, ³J_{HH}=8.4 Hz, 4H), 1.98 (q, ³J_{HH}=6.3 Hz, 8H), 1.56 (m, 6H), 1.29-1.23 (m, 46H), 0.84 (t, ³J_{HH}=6.4 Hz, 9H). ¹³C-NMR (125 MHz, CDCl₃) δ 173.5, 173.1, 130.2, 129.8, 124.6, 122.1, 119.5, 116.9, 71.7, 70.6, 70.5, 70.4, 70.2, 70.1, 70.0, 69.9, 69.8, 69.6, 69.5, 67.9, 67.8, 66.2, 66.1, 65.7, 65.6, 61.8, 54.7, 34.3, 34.2, 32.1, 31.9, 29.9, 29.7, 29.5, 29.4, 29.3, 29.2, 27.4, 27.3, 25.9, 25.0, 22.9, 22.8, 14.3, 14.2. ³¹P-NMR (162 MHz, CDCl₃) δ −1.84, −1.87. ESI-MS (m/z): calc. for C₅₀H₉₇NO₈P(C₂H₄O), 1178.88. found 1178.8. FT-IR (thin film) ν 2922, 2853, 1741, 1465, 1258, 1225, 1152, 1097, 1030, 983, 639.

### Compound PP514.

This compound was prepared from DOPC (230 mg, 0.29 mmol) and 2b (239 mg, 0.75 mmol) in dry CHCl₃ (8 mL). Column chromatography yielded the expected compound PP514 in 96% yield (268 mg, 0.28 mmol) as a waxy solid (mixture of 2 diastereomers). TLC R_{f} 0.32 (CH₂Cl₂/MeOH 90:10). ¹H-NMR (400 MHz, CDCl₃) δ 5.33-5.30 (m, 5H), 4.49 (br, 2H), 4.34-4.04 (m, 6H), 3.85 (m, 2H), 3.29 (s, 9H), 2.32-2.27 (m, 4H), 2.00-1.96 (m, 8H), 1.68-1.56 (m, 6H), 1.27-1.23 (m, 58H), 0.86 (t, ³J_{HH}=6.6 Hz, 9H). ¹³C-NMR (100 MHz, CDCl₃) δ 173.5, 173.2, 130.2, 129.8, 69.7, 69.5, 69.4, 66.2, 65.9, 65.8, 61.8, 61.7, 61.5, 54.8, 34.3, 34.2, 32.1, 30.4, 30.3, 29.9 (2C), 29.8 (2C), 29.7, 29.5, 29.4, 29.3, 29.2, 27.4, 25.5, 25.1, 22.9, 14.3. ³¹P-NMR (162 MHz, CDCl₃) δ −1.73, −1.77. ESI-MS (m/z): calc. for C₅₆H₁₀₉NO₈P 954.79. found 954.7. FT-IR (thin film) ν 2923, 2851, 1744, 1470, 1262, 1223, 1098, 985, 638.

### Compound PP531.

This compound was prepared from DOPC (102 mg, 0.13 mmol) and 2c (122 mg, 0.34 mmol) in dry CHCl₃ (5 mL). Column chromatography yielded the expected compound PP531 in 77% yield (100 mg, 0.10 mmol) as a waxy solid (mixture of 2 diastereomers). TLC R_{f} 0.29 (CH₂Cl₂/MeOH 90:10). ¹H-NMR (400 MHz, CDCl₃) δ 5.33-5.27 (m, 5H), 4.51 (br, 2H), 4.33-4.08 (m, 6H), 3.85 (m, 2H), 3.60 (m, 2H), 3.43 (t, ³J_{HH}=6.9 Hz, 2H), 3.29 (s, 9H), 2.32-2.26 (m, 4H), 2.00-1.96 (m, 8H), 1.58-1.52 (m, 6H), 1.27-1.23 (m, 58H), 0.86 (t, ³J_{HH}=6.7 Hz, 9H). ¹³C-NMR (125 MHz, CDCl₃) δ 173.5, 173.2, 130.2, 129.8, 69.6, 69.5, 69.4, 69.3, 69.2, 68.2, 68.1, 66.3, 65.9, 65.7, 61.8, 61.6, 54.7, 34.3, 34.1, 32.1, 29.9, 29.8, 29.7, 29.5, 29.4, 29.3 (2C), 29.2, 27.4 (2C), 26.2, 25.8, 25.0, 22.8, 14.3. ³¹P-NMR (162 MHz, CDCl₃) δ −1.89, −1.95. ESI-MS (m/z): calc. for C₅₆H₁₀₉NO₈P(C₂H₄O)₁ 998.81. found 998.8. FT-IR (thin film) ν 2923, 2853, 1743, 1465, 1262, 1225, 1162, 1031, 984, 744, 639.

### Compound PP529.

This compound was prepared from DOPC (34 mg, 43.30 μmol) and 2d (51 mg, 0.11 mmol) in dry CHCl₃ (3 mL). Column chromatography yielded the expected compound PP529 in 57% yield (27 mg, 24.84 grind) as a clear oil (mixture of 2 diastereomers). TLC R_{f} 0.41 (CH₂Cl₂/MeOH 90:10). ¹H-NMR (400 MHz, CDCl₃) δ 5.32-5.22 (m, 5H), 4.54 (m, 2H), 4.32-4.12 (m, 6H), 3.84 (br, 2H), 3.71-3.55 (m, 10H), 3.43 (t, ³J_{HH}=7.0 Hz, 2H), 3.27 (s, 9H), 2.30-2.26 (m, 4H), 2.00-1.96 (m, 8H), 1.57-1.54 (m, 6H), 1.27-1.21 (m, 58H), 0.86 (t, ³J_{HH}=6.7 Hz, 9H). ¹³C-NMR (100 MHz, CDCl₃) δ 173.6, 173.2, 130.2, 129.9, 71.8, 71.7, 70.4, 70.3, 70.1, 70.0, 69.7, 69.6, 69.5, 69.3, 68.1, 67.9, 67.8, 66.4, 66.3, 65.8, 65.7, 65.3, 63.4, 61.9, 61.8, 54.6, 34.3, 34.2, 32.1, 29.9, 29.8, 29.7, 29.6, 29.5, 29.4, 29.3, 29.2, 29.1, 27.4, 26.2, 26.1, 25.8, 25.1, 25.0, 22.9, 22.7, 14.3. ³¹P-NMR (162 MHz, CDCl₃) δ −1.72, −1.77. ESI-MS (m/z): calc. for C₅₆H₁₀₉NO₈P(C₂H₄O)₃ 1086.87. found 1086.8. FT-IR (thin film) ν 2920, 2862, 1742, 1465, 1255, 1231, 1150, 1098, 1031, 984.

### Compound PP415.

This compound was prepared from DOPC (234 mg, 0.297 mmol) and 2e (390 mg, 0.78 mmol) in dry CHCl₃ (10 mL). Column chromatography yielded the expected compound PP415 in 53% yield (200 mg, 0.156 mmol) as a clear oil (mixture of 2 diastereomers). TLC R_{f} 0.34 (CH₂Cl₂/MeOH 90:10). ¹H-NMR (400 MHz, CDCl₃) δ 5.33-5.30 (m, 4H), 5.22 (br s, 1H), 4.53 (br s, 2H), 4.31-4.10 (m, 6H), 3.85 (m, 2H), 3.69-3.52 (m, 16H), 3.41 (t, ³J_{HH}=6.9 Hz, 2H), 3.29 (s, 9H), 2.29 (q, ³J_{HH}=8.3 Hz, 4H), 1.99 (q, ³J_{HH}=6.3 Hz, 8H), 1.56 (m, 6H), 1.30-1.23 (m, 58H), 0.85 (t, ³J_{HH}=6.5 Hz, 9H). ¹³C-NMR (100 MHz, CDCl₃) δ 173.5, 173.1, 130.2, 129.8, 71.7, 70.6, 70.5, 70.4, 70.2, 70.1, 70.0, 69.9, 69.6, 69.5, 68.0, 67.9, 66.3, 65.9, 65.8, 61.8, 54.7, 53.6, 34.3, 34.2, 32.1, 29.9, 29.8, 29.7, 29.5, 29.4, 29.3, 29.2, 27.4, 26.3, 25.0, 22.9, 14.3. ³¹P-NMR (162 MHz, CDCl₃) δ −1.84, −1.88. ESI-MS (m/z): calc. for C₅₆H₁₀₉NO₈P(C₂H₄O)₄ 1130.89. found 1130.8. FT-IR (thin film) ν 2922, 2853, 1742, 1465, 1258, 1225, 1157, 1097, 1030, 983, 639.

### Compound PP418.

This compound was prepared from DOPC (245 mg, 0.31 mmol) and 2f (585 mg, 0.82 mmol) in dry CHCl₃ (10 mL). Column chromatography yielded the expected compound PP418 in 49% yield (231 mg, 0.154 mmol) as a clear oil (mixture of 2 diastereomers). TLC R_{f} 0.37 (CH₂Cl₂/MeOH 90:10). ¹H-NMR (400 MHz, CDCl₃) δ 5.33-5.29 (m, 4H), 5.23-5.20 (br s, 1H), 4.54 (br s, 2H), 4.31-4.12 (m, 6H), 3.87 (m, 2H), 3.69-3.53 (m, 26H), 3.41 (t, ³J_{HH}=6.9 Hz, 2H), 3.28 (s, 9H), 2.29 (q, ³J_{HH}=9.5 Hz, 4H), 1.98 (q, ³J_{HH}=5.8 Hz, 8H), 1.56 (m, 6H), 1.29-1.23 (m, 58H), 0.84 (t, ³J_{HH}=7.1 Hz, 9H). ¹³C-NMR (125 MHz, CDCl₃) δ 173.5, 173.1, 130.2, 129.9, 124.6, 122.1, 119.5, 116.9, 71.7, 70.6, 70.5, 70.4, 70.3, 70.2, 70.1, 70.0, 69.9, 69.6, 69.5, 68.0, 67.9, 66.2, 66.2, 65.8, 61.9, 61.8, 54.7, 54.6, 34.3, 34.2, 32.1, 29.9, 29.8, 29.7, 29.5, 29.4, 29.3, 29.2, 27.4, 27.3, 26.3, 25.0, 22.9, 22.8, 14.3, 14.2. ³¹P-NMR (162 MHz, CDCl₃) δ −1.80, −1.84. ESI-MS (m/z): calc. for C₅₆H₁₀₉NO₈P(C₂H₄O)₇ 1262.97. found 1262.9. FT-IR (thin film) ν 2922, 2853, 1742, 1458, 1258, 1225, 1155, 1097, 1030, 983, 638.

### Compound PP427.

This compound was prepared from DOPC (271 mg, 0.34 mmol) and 2g (1.20 g, 0.90 mmol) in dry CHCl₃ (10 mL). Column chromatography yielded the expected compound PP427 in 56% yield (400 mg, 0.19 mmol) as a clear oil (mixture of 2 diastereomers). TLC R_{f} 0.28 (CH₂Cl₂/MeOH 90:10). ¹H-NMR (300 MHz, CDCl₃) δ 5.32-5.29 (m, 4H), 5.23-5.21 (br s, 1H), 4.53 (br s, 2H), 4.32-4.11 (m, 6H), 3.88 (m, 2H), 3.62 (m, 94H), 3.41 (t, ³J_{HH}=6.9 Hz, 2H), 3.28 (s, 9H), 2.29 (q, ³J_{HH}=7.3 Hz, 4H), 1.97 (q, ³J_{HH}=6.2 Hz, 8H), 1.56 (m, 6H), 1.29-1.23 (m, 58H), 0.85 (t, ³J_{HH}=6.4 Hz, 9H). ¹³C-NMR (125 MHz, CDCl₃) δ 173.5, 173.1, 130.2, 129.8, 124.6, 122.1, 119.5, 116.9, 71.7, 70.7, 70.6, 70.5, 70.0, 69.9, 69.6, 69.5, 67.9, 67.8, 66.2, 66.1, 65.6, 61.9, 61.8, 54.6, 34.3, 32.1, 29.9, 29.8, 29.7, 29.5, 29.4, 29.3, 29.2, 27.4, 27.3, 26.3, 25.0, 22.9, 22.8, 14.3. ³¹P-NMR (162 MHz, CDCl₃) δ −1.93, −1.96. ESI-MS (m/z): calc. for C₅₆H₁₀₉NNaO₈P(C₂H₄O)₂₃ 995.19. found 995.6. FT-IR (thin film) ν 2922, 2854, 1742, 1458, 1350, 1258, 1225, 1097, 1030, 985, 845, 730, 638.

### Compound PP426.

This compound was prepared from DOPC (247 mg, 0.31 mmol) and 2h (655 mg, 0.82 mmol) in dry CHCl₃ (10 mL). Column chromatography yielded the expected compound PP426 in 55% yield (273 mg, 0.17 mmol) as a clear oil (mixture of 2 diastereomers). TLC R_{f} 0.29 (CH₂Cl₂/MeOH 90:10). ¹H-NMR (300 MHz, CDCl₃) δ 5.31-5.29 (m, 4H), 5.23-5.20 (br s, 1H), 4.53 (br s, 2H), 4.31-4.10 (m, 6H), 3.84 (m, 2H), 3.69-3.52 (m, 30H), 3.41 (t, ³J_{HH}=6.8 Hz, 2H), 3.28 (s, 9H), 2.29 (q, ³J_{HH}=7.3 Hz, 4H), 1.98 (m, 8H), 1.56 (m, 6H), 1.27-1.22 (m, 70H), 0.84 (t, ³J_{HH}=6.3 Hz, 9H). ¹³C-NMR (125 MHz, CDCl₃) δ 173.5, 173.1, 130.2, 129.8, 124.6, 122.1, 119.5, 116.9, 71.7, 70.6, 70.5, 70.1, 69.9, 69.8, 69.5, 67.9, 67.8, 66.2, 66.1, 65.7, 61.9, 61.8, 54.6, 34.3, 34.2, 32.1, 29.9, 29.8, 29.7, 29.6, 29.5, 29.4, 29.3, 29.2, 27.4, 27.3, 26.2, 25.0, 22.9, 14.3. ³¹P-NMR (162 MHz, CDCl₃) δ −1.90, −1.94. ESI-MS (m/z): calc. for C₆₂H₁₂₁NO₈P(C₂H₄O)₈ 1391.09. found 1391.0. FT-IR (thin film) ν 2922, 2853, 1742, 1465, 1259, 1225, 1144, 1098, 1030, 982, 639.

### Compound PP522.

This compound was prepared from DOPC (40 mg, 50.95 μmol) and 2i (105 mg, 0.13 mmol) in dry CHCl₃ (3 mL). Column chromatography yielded the expected compound PP522 in 66% yield (52 mg, 32.84 pmol) as a clear oil (mixture of 2 diastereomers). TLC R_{f} 0.35 (CH₂Cl₂/MeOH 90:10). ¹H-NMR (300 MHz, CDCl₃) δ 5.33-5.20 (m, 7H), 4.56 (m, 2H), 4.33-4.09 (m, 6H), 3.91 (br, 2H), 3.74-3.56 (m, 30H), 3.42 (t, ³J_{HH}=6.8 Hz, 2H), 3.29 (s, 9H), 2.31-2.28 (m, 4H), 2.01-1.96 (m, 12H), 1.58-1.52 (m, 6H), 1.27-1.23 (m, 62H) 0.86 (t, ³J_{HH}=6.7 Hz, 9H). ¹³C-NMR (125 MHz, CDCl₃) δ 173.5, 173.2, 130.2 (2C), 129.9, 129.8, 71.7, 71.6, 70.3, 70.2, 70.1, 70.0, 69.9, 69.8, 69.7, 69.6, 69.5, 67.9, 66.3, 66.2, 65.7, 65.6, 65.3, 62.0, 61.8, 54.7, 54.6, 34.3, 34.2, 32.8, 32.1, 29.9, 29.7, 29.6, 29.4, 29.3, 29.2, 27.4, 26.2, 25.1, 22.8, 14.3. ³¹P-NMR (162 MHz, CDCl₃) δ −1.69, −1.74. ESI-MS (m/z): calc. for C₆₂H₁₁₉NO₈P(C₂H₄O)₇ 1345.05. found 1345.0. FT-IR (thin film) ν 2922, 2854, 1742, 1465, 1260, 1225, 1157, 1103, 1031, 985, 951, 743, 639.

### Synthesis of Cationic Lipid PP430

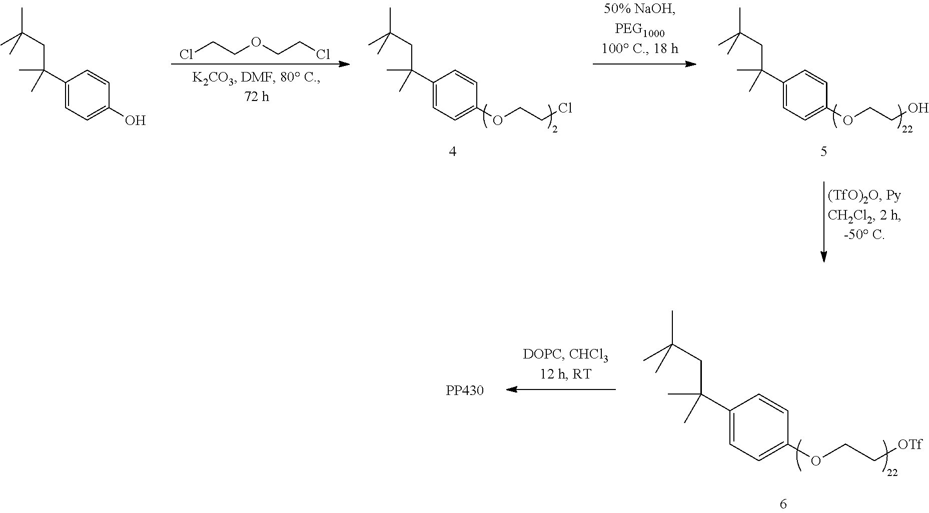

### Compound 4.

1-chloro-2-(2-chloroethoxy)ethane (2.34 mL, 20 mmol) was added dropwise to a mixture of K₂CO₃ (1.38 g, 10 mmol) and 4-(2,4,4-trimethylpentan-2-yl)phenol (2.06 g, 10 mmol) in dry DMF (30 mL) under argon at 80° C. The reaction mixture was cooled down, diluted with water (20 mL) and extracted trice with ethyl acetate (30 mL). Combined organic layers were dried over anhydrous magnesium sulfate, filtered and concentrated under reduced pressure. The crude product was then purified by flash column chromatography (EP/CH₂Cl₂ 70/30) to provide the expected compound as a clear oil in 62% yield (1.95 g, 6.23 mmol). TLC R_{f} 0.68 (EP/CH₂Cl₂ 1:1). ¹H-NMR (400 MHz, CDCl₃) δ 7.24 (d, ³J_{HH}=8.8 Hz, 2H), 6.81 (d, ³J_{HH}=8.8 Hz, 2H), 4.10 (t, ³J_{HH}=4.8 Hz, 2H), 3.78 (m, 4H), 3.62 (m, 2H), 1.67 (s, 2H), 1.32 (s, 6H), 0.68 (s, 9H). ¹³C-NMR (100 MHz, CDCl₃) δ 156.5, 142.8, 127.3, 114.0, 71.7, 71.6, 57.2, 42.9, 42.8, 38.2, 32.5, 31.9, 31.8.

### Compound 5.

To a solution of PEG₁₀₀₀ (31.25 g, 31.25 mmoles) was added 50% NaOH (31.25 mmol). The mixture was refluxed at 100° C. for 30 min until the color changed to brown. Compound 4 (1.95 g, 6.25 mmol) was then added and the resulting mixture was heated for additional 18 h. The reaction mixture was cooled down and diluted with water (30 mL). The product was extracted with CH₂Cl₂ (2×50 mL). Organic layer was concentrated under reduced pressure and diluted in EtOAc (150 mL). The mixture was washed twice with water (300 mL), dried over anhydrous magnesium sulfate, filtered and concentrated under reduced pressure. The crude product was then purified by flash column chromatography (CH₂Cl₂/MeOH 100:0 to 95:5) to provide the expected compound as yellow oil in 18% yield (1.22 g, 1.12 mmol). TLC R_{f} 0.35 (CH₂Cl₂/MeOH 90:10). ¹H-NMR (300 MHz, CDCl₃) δ 7.19 (d, ³J_{HH}=8.2 Hz, 2H), 6.76 (d, ³J_{HH}=8.2 Hz, 2H), 4.04 (t, ³J_{HH}=4.8 Hz, 2H), 3.78 (t, ³J_{HH}=4.9 Hz, 2H), 3.65-3.58 (m, 76H), 1.63 (s, 2H), 1.27 (s, 6H), 0.64 (s, 9H). ¹³C-NMR (100 MHz, CDCl₃) δ 156.4, 142.4, 127.0, 113.8, 72.6, 70.8, 70.7, 70.6, 70.4, 69.9, 67.4, 61.8, 57.1, 37.9, 32.4, 31.8, 31.7.

### Compound 6.

Triflic anhydride (52 μL, 0.309 mmol) was added dropwise to a solution of 5 (280 mg, 0.26 mmol) and pyridine (25 μL, 0.309 mmol) in freshly distilled CH₂Cl₂ (3.0 mL) at −50° C. The reaction mixture was stirred under argon at −50° C. for 2 h before it was poured into ice-cold water (20 mL). The organic layer was separated and washed twice with cold water (2×10 mL). The organic layer was dried over anhydrous magnesium sulfate, filtered and concentrated in vacuo at 20° C. The corresponding trifluoromethanesulfonyl ester 6 was obtained as a clear viscous oil (0.26 g, 82%) and used in the next step without further purification. Due to relative instability of the compound, ¹H NMR spectrum was recorded immediately after isolation. ¹H-NMR (300 MHz, CDCl₃) δ 7.22 (d, ³J_{HH}=8.2 Hz, 2H), 6.79 (d, ³J_{HH}=8.4 Hz, 2H), 4.62 (t, ³J_{HH}=4.1 Hz, 2H), 4.08 (t, ³J_{HH}=4.5 Hz, 2H), 3.84-3.58 (m, 64H), 1.67 (s, 2H), 1.31 (s, 6H), 0.68 (s, 9H).

### Compound PP430.

To a solution of DOPC (63 mg, 83 μmol) in dry CHCl₃ (1 mL) was added a solution of freshly prepared trifluoromethanesulfonyl ester 6 (260 mg, 0.21 mmol) in dry CHCl₃ (2 mL). The resulting reaction mixture was stirred at room temperature for 20 h under argon. The reaction mixture was concentrated under reduced pressure at room temperature. The crude product was then purified by flash column chromatography using (CH₂Cl₂/MeOH 98:2) as eluent to provide the expected compound PP430 in 16% yield (25 mg, 12.5 μmol), and as a clear oil (mixture of 2 diastereomers). TLC R_{f} 0.37 (CH₂Cl₂/MeOH 90:10). ¹H-NMR (400 MHz, CDCl₃) δ 7.22 (d, ³J_{HH}=8.2 Hz, 2H), 6.78 (d, ³J_{HH}=8.1 Hz, 2H), 5.31-5.29 (m, 4H), 5.23-5.21 (br s, 1H), 4.54 (br s, 2H), 4.30-4.07 (m, 8H), 3.83-3.62 (m, 78H), 3.29 (s, 9H), 2.31 (q, ³J_{HH}=7.4 Hz, 4H), 1.99 (m, 8H), 1.67 (s, 2H), 1.58 (m, 4H), 1.30-1.24 (m, 46H), 0.85 (t, ³J_{HH}=6.7 Hz, 9H), 0.68 (s, 9H). ¹³C-NMR (125 MHz, CDCl₃) δ 173.5, 173.1, 156.6, 143.6, 130.2, 129.9, 127.2, 114.0, 70.9, 70.8, 70.7, 70.6, 70.1, 70.0, 69.9, 69.6, 69.5, 68.0, 67.9, 67.5, 66.3, 66.2, 65.8, 62.0, 61.9, 61.8, 57.2, 54.7, 38.2, 34.3, 34.2, 32.5, 29.9, 29.8, 29.6, 29.5, 29.4, 29.3, 29.2, 27.4, 27.3, 25.0, 22.8, 14.3. ³¹P-NMR (162 MHz, CDCl₃) δ −1.96, −1.98. ESI-MS (m/z): calc. for C₅₈H₁₀₅NNaO₈P(C₂H₄O)₂₀ 939.15. found 939.1. FT-IR (thin film) ν 2922, 2854, 1740, 1511, 1463, 1255, 1224, 1100, 1030, 985, 831, 638.

### Synthesis of Lipid PP431

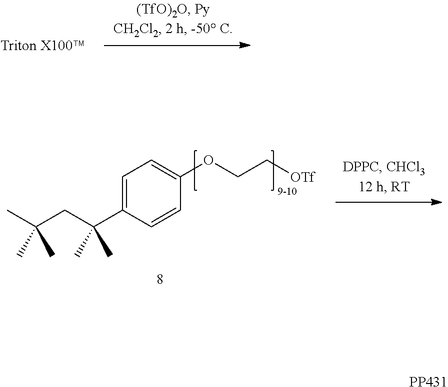

### Compound 8.

Triflic anhydride (207 μL, 1.20 mmol) was added dropwise to a solution of TX100 (602 mg, 1.00 mmol) and pyridine (96 μL, 1.20 mmol) in freshly distilled CH₂Cl₂ (6.0 mL) at −50° C. The reaction mixture was stirred under argon at −50° C. for 2 h before it was poured into ice-cold water (20 mL). The organic layer was separated and washed twice with cold water (2×10 mL). The organic layer was dried over anhydrous magnesium sulfate, filtered and concentrated in vacuo at 20° C. The corresponding trifluoromethanesulfonyl ester 8 was obtained as a clear viscous oil (616 mg, 84%) and used in the next step without further purification. Due to relative instability of the compound, ¹H NMR spectrum was recorded immediately after isolation. ¹H-NMR (300 MHz, CDCl₃) δ 7.26 (d, ³J_{HH}=8.9 Hz, 2H), 6.81 (d, ³J_{HH}=8.9 Hz, 2H), 4.63 (t, ³J_{HH}=4.5 Hz, 2H), 4.11 (t, ³J_{HH}=4.5 Hz, 2H), 3.86-3.64 (m, 36H), 1.69 (s, 2H), 1.33 (s, 6H), 0.71 (s, 9H).

### Compound PP431.

To a solution of DPPC (234 mg, 0.32 mmol) in dry CHCl₃ (5 mL) was added a solution of freshly prepared TX100 trifluoromethanesulfonyl ester 8 (616 mg, 0.84 mmol) in dry CHCl₃ (5 mL). The resulting reaction mixture was stirred at room temperature for 20 h under argon. The reaction mixture was concentrated under reduced pressure at room temperature. The crude product was then purified by flash column chromatography using (CH₂Cl₂/MeOH 95:5) as eluent to provide the expected compound PP431 in 37% yield (180 mg, 118 pmol), and as a clear oil (mixture of 2 diastereomers). TLC R_{f} 0.34 (CH₂Cl₂/MeOH 90:10). ¹H-NMR (400 MHz, CDCl₃) δ 7.22 (d, ³J_{HH}=8.2 Hz, 2H), 6.79 (d, ³J_{HH}=8.1 Hz, 2H), 5.22 (br, 1H), 4.53 (br, 2H), 4.32-4.07 (m, 8H), 3.85-3.61 (m, 34H), 3.28 (s, 9H), 2.29 (m, 4H), 1.67 (s, 2H), 1.57 (m, 4H), 1.30-1.23 (m, 54H), 0.85 (t, ³J_{HH}=6.6 Hz, 9H), 0.68 (s, 9H). ¹³C-NMR (125 MHz, CDCl₃) δ 173.5, 173.1, 156.4, 142.8, 127.2, 124.6, 122.0, 119.5, 117.0, 113.9, 70.6, 70.3, 70.2, 70.1, 70.0, 69.9, 69.8, 69.5, 67.9, 67.4, 66.2, 65.6, 65.5, 61.9, 61.8, 57.2, 54.6, 38.1, 34.3, 34.2, 32.5, 32.1, 31.9, 31.8, 29.9, 29.8, 29.7, 29.6, 29.5, 29.3, 25.0, 22.9, 14.3. ³¹P-NMR (162 MHz, CDCl₃) δ −1.81, −1.87. ESI-MS (m/z): calc. for C₅₆H₁₀₅NO₈P(C₂H₄O)₉ 1362.99. found 1362.9. FT-IR (thin film) ν 2922, 2853, 1741, 1511, 1466, 1256, 1225, 1150, 1100, 1030, 983, 829, 639.

Synthesis of pH-Sensitive Analogues of PP415 (MR22, MR25, MR26, MR28)

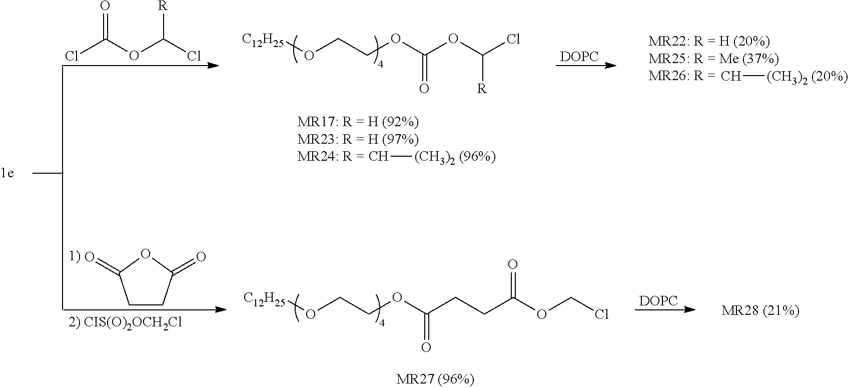

### Conjugate MR22.

To a solution of 1e (2.00 g, 5.40 mmol) and pyridine (547 μL, 6.79 mmol) in freshly distilled CH₂Cl₂ (40.0 mL) was added chloromethylchloroformate (522 μL, 5.94 mmol). The reaction mixture was stirred at room temperature for 18 h under a positive argon atmosphere, whereupon the mixture was quenched with water (50.0 mL) and extracted with EtOAc (2×100 mL). The organic layer was dried over Na₂SO₄, filtered, and volatiles were removed under reduced pressure. Compound MR17 was recovered as a clear oil (2.26 g, 92%) and was used in the next step without further purification. TLC R_{f} 0.60 (CH₂Cl₂/MeOH 90:10). ¹H-NMR (400 MHz, CDCl₃) δ 5.68 (s, 2H), 4.33-4.30 (m, 2H), 3.70-3.68 (m, 2H), 3.60-3.57 (m, 16H), 3.53-3.50 (m, 2H), 1.52-1.50 (m, 2H), 1.26-1.20 (m, 18H), 0.82 (t, ³J_{HH}=6.6 Hz, 3H). ¹³C-NMR (100 MHz, CDCl₃) δ 153.5, 72.3, 71.6, 70.8, 70.7, 70.1, 66.7, 32.0, 29.8, 29.6, 26.2, 22.7, 14.2. FT-IR (thin film) ν 2922, 2854, 2360, 2341, 1766, 1250, 1108.

To a solution of DOPC (421 mg, 0.536 mmol) in freshly distilled CHCl₃ (10 mL) was added a solution of compound MR17 (1.95 g, 4.28 mmol) in dry CHCl₃ (5 mL). The resulting reaction mixture was heated at reflux for 20 h under a positive argon atmosphere, then cooled down and concentrated under reduced pressure at 20-25° C. The residue was purified over silica gel by flash chromatography (CH₂Cl₂/MeOH 88:12 to 80:20) to yield compound MR22 (130 mg, 20%) as a waxy solid (mixture of 2 diastereomers). TLC R_{f} 0.53 (CH₂Cl₂/MeOH 90:10). ¹H-NMR (400 MHz, CDCl₃) δ 5.69-5.59 (m, 2H), 5.35-5.25 (m, 4H), 5.24-5.17 (m, 1H), 5.63-5.61 (br, 2H), 4.39-4.06 (m, 8H), 3.71 (t, ³J_{HH}=4.7 Hz, 2H), 3.65-3.50 (m, 18H), 3.46 (s, 9H), 3.40 (t, ³J_{HH}=6.9 Hz, 2H), 2.36-2.40 (m, 4H), 2.05-1.90 (m, 8H), 1.65-1.47 (m, 2H), 1.36-1.15 (m, 58H), 1.05 (t, ³J_{HH}=6.6 Hz, 6H), 0.84 (t, J=6.7 Hz, 9H). ¹³C-NMR (100 MHz, CDCl₃) δ 173.4, 173.0, 153.5, 130.2, 129.9, 86.2, 71.7, 70.7, 70.2, 69.4, 68.7, 68.2, 66.8, 66.7, 65.5, 65.4, 62.4, 61.6, 54.7, 34.2, 32.1, 29.9, 29.8, 29.7, 29.6, 29.5, 29.4, 29.3, 29.2, 27.4, 27.3, 26.2, 25.1, 22.9, 14.2. ³¹P-NMR (162 MHz, CDCl₃) δ −3.2, −3.3. ESI-MS (m/z): calc. for C₅₈H₁₁₁NO₁₁P(C₂H₄O)₄ 1204.89. found 1204.8. FT-IR (thin film) ν 2922, 2852, 2360, 2341, 1741, 1260.

### Conjugate MR25.

To a solution of 1e (2.00 g, 5.40 mmol) and pyridine (547 μL, 6.79 mmol) in freshly distilled CH₂Cl₂ (40.0 mL) was added chloroethylchloroformate (641 μL, 5.94 mmol). The reaction mixture was stirred at room temperature for 18 h under a positive argon atmosphere, whereupon the mixture was quenched with water (50.0 mL) and extracted with EtOAc (2×100 mL). The organic layer was dried over Na₂SO₄, filtered, and volatiles were removed under reduced pressure. Compound MR23 was recovered as a clear oil (2.33 g, 92%) and was used in the next step without further purification. TLC R_{f} 0.55 (CH₂Cl₂/MeOH 90:10). ¹H-NMR (400 MHz, CDCl₃) δ 6.4 (q, ³J_{HH}=5.8 Hz, 1H), 4.34-4.32 (m, 2H), 3.72 (t ³J_{HH}=4.1 Hz, 2H), 3.63-3.54 (m, 16H), 3.42 (t, ³J_{HH}=6.8 Hz, 2H), 1.81 (d, ³J_{HH}=5.8 Hz, 3H), 1.57-1.53 (m, 2H), 1.29-1.23 (m, 18H), 0.86 (t, ³J_{HH}=6.7 Hz, 3H). ¹³C-NMR (100 MHz, CDCl₃) δ 153.1, 84.8, 71.8, 70.9, 70.8, 70.2, 68.9, 68.1, 32.1, 29.8, 29.7, 29.6, 26.3, 25.4, 22.9, 14.3. To a solution of DOPC (488 mg, 0.621 mmol) in freshly distilled CHCl₃ (10 mL) was added a solution of compound MR23 (2.33 g, 4.96 mmol) in dry CHCl₃ (5 mL). The resulting reaction mixture was heated at reflux for 20 h under a positive argon atmosphere, then cooled down and concentrated under reduced pressure at 20-25° C. The residue was purified over silica gel by flash chromatography (CH₂Cl₂/MeOH 88:12 to 80:20) to yield compound MR25 (290 mg, 37%) as a waxy solid (mixture of 4 diastereomers). TLC R_{f} 0.53 (CH₂Cl₂/MeOH 90:10). ¹H-NMR (400 MHz, CDCl₃) δ 6.41-6.21 (m, 1H), 5.37-5.24 (m, 4H), 5.24-5.17 (m, 1H), 4.54 (br, 2H), 4.38-4.05 (m, 8H), 3.83-3.86 (m, 2H), 3.65-3.50 (m, 18H), 3.46 (s, 9H), 3.40 (t, ³J_{HH}=6.9 Hz, 2H), 2.36-2.23 (m, 4H), 2.05-1.90 (m, 8H), 1.65-1.47 (m, 9H), 1.36-1.15 (m, 58H), 0.84 (t, ³J_{HH}=6.7 Hz, 9H). ¹³C-NMR (100 MHz, CDCl₃) δ 173.4, 173.0, 153.1, 95.7, 95.3, 71.1, 70.8, 70.7, 70.2, 69.6, 69.5, 69.3, 69.2, 68.8, 68.1, 66.8, 68.1, 66.8, 66.7, 66.6, 66.4, 66.3, 66.2, 65.5, 65.4, 62.5, 62.2, 61.7, 61.6, 61.5, 54.6, 34.3, 34.2, 32.1, 30.4, 29.9, 29.6, 29.4, 29.3, 27.4, 26.3, 25.2, 25.0, 23.7, 22.9, 21.4, 20.9, 14.3. ³¹P-NMR (162 MHz, CDCl₃) δ −5.3, −5.5, −5.7, −5.9. ESI-MS (m/z): calc. for C₅₉H₁₁₃NO₁₁P(C₂H₄O)₄ 1218.91. found 1218.9. FT-IR (thin film) ν 2922, 2852, 1743, 1457, 1264, 1108, 1084, 971, 669.

### Conjugate MR26.

To a solution of 1e (2.00 g, 5.40 mmol) and pyridine (547 μL, 6.79 mmol) in freshly distilled CH₂Cl₂ (40.0 mL) was added 1-chloro-2-methylpropyl chloroformate (884 μL, 5.94 mmol). The reaction mixture was stirred at room temperature for 18 h under a positive argon atmosphere, whereupon the mixture was quenched with water (50.0 mL) and extracted with EtOAc (2×100 mL). The organic layer was dried over Na₂SO₄, filtered, and volatiles were removed under reduced pressure. Compound MR24 was recovered as a clear oil (2.58 g, 96%) and was used in the next step without further purification. TLC R_{f} 0.50 (CH₂Cl₂/MeOH 90:10). ¹H-NMR (400 MHz, CDCl₃) δ 6.16 (d, ³J_{HH}=4.5 Hz, 1H), 4.34-4.32 (m, 2H), 3.73 (t, ³J_{HH}=4.5 Hz, 2H), 3.64-3.54 (m, 16H), 3.42 (t, ³J_{HH}=6.8 Hz, 2H), 2.23-2.16 (m, 1H), 1.63-1.48 (m, 2H), 1.29-1.23 (m, 18H), 1.05 (t, ³J_{HH}=6.6 Hz, 6H), 0.86 (t, ³J_{HH}=6.7 Hz). To a solution of DOPC (533 mg, 0.679 mmol) in freshly distilled CHCl₃ (10 mL) was added a solution of compound MR24 (2.70 g, 5.43 mmol) in dry CHCl₃ (5 mL). The resulting reaction mixture was heated at reflux for 20 h under a positive argon atmosphere, then cooled down and concentrated under reduced pressure at 20-25° C. The residue was purified over silica gel by flash chromatography (CH₂Cl₂/MeOH 88:12 to 80:20) to yield compound MR26 (171 mg, 20%) as a waxy solid (mixture of 4 diastereomers). TLC R_{f} 0.53 (CH₂Cl₂/MeOH 90:10). ¹H-NMR (400 MHz, CDCl₃) δ 6.16-6.10 (m, 1H), 5.35-5.20 (m, 5H), 4.57 (br, 2H), 4.47-4.12 (m, 8H), 3.84-3.72 (m, 2H), 3.70-3.65 (m, 18H), 3.46 (t, ³J_{HH}=6.1 Hz, 2H), 3.25 (s, 9H), 2.40-2.29 (m, 4H), 2.18-2.07 (m, 1H), 2.06-1.95 (m, 8H), 1.69-1.50 (m, 6H), 1.39-1.22 (m, 58H), 1.08-0.99 (m, 6H), 0.88 (t, ³J_{HH}=6.7 Hz, 9H). ¹³C-NMR (100 MHz, CDCl₃) δ 174.7, 154.7, 130.9, 130.6, 101.7, 101.6, 78.5, 72.4, 71.4, 71.3, 71.0, 70.7, 70.6, 69.7, 69.6, 68.9, 67.6, 66.7, 66.6, 54.8, 35.0, 34.9, 33.9, 32.9, 30.9, 30.6, 30.4, 30.2, 30.1, 28.1, 27.1, 25.9, 23.6, 18.4, 16.8, 16.7, 16.4, 14.6. ³¹P-NMR (162 MHz, CDCl₃) δ −4.7, −4.8, −4.9, −5.1. ESI-MS (m/z): calc. for C₆₁H₁₁₇NO₁₁P(C₂H₄O)₄ 1246.94. found 1246.9. FT-IR (thin film) ν 2925, 2854, 1745, 1510, 1258, 1160, 1092, 1031, 637.

### Conjugate MR28.

A mixture of 1e (4.00 g, 10.86 mmol) and succinic anhydride (3.26 g, 32.6 mmol) in dry pyridine (30 mL) was stirred at room temperature for 18 h. Volatiles were then removed under reduced pressure. The crude residue was then stirred in 5% NaHCO₃ aqueous solution (20 mL) for 30 minutes, acidified to pH 3 with HCl 12M and extracted twice with CH₂Cl₂/MeOH (2:1, 50 mL). Combined organic layers were dried over Na₂SO₄, filtered and concentrated under reduced pressure. The crude residue was purified by flash chromatography over silica gel (CH₂Cl₂/MeOH 95:5) to yield the intermediate hemisuccinate derivative as a waxy solid (4.76 g, 95%). TLC R_{f} 0.46 (CH₂Cl₂/MeOH 90:10). ¹H-NMR (400 MHz, CDCl₃) δ 4.25-4.23 (m, 2H), 3.67-3.40 (m, 16H), 3.44-3.40 (t, ³J_{HH}=8.8 Hz, 2H), 2.63 (m, 2H), 1.56-1.53 (m, 2H), 1.27-1.23 (m, 18H), 0.85 (t, ³J_{HH}=6.5 Hz, 3H). ¹³C-NMR (100 MHz, CDCl₃) δ 175.9, 172.2, 71.6, 70.7, 70.6, 70.1, 69.1, 63.9, 32.0, 29.7, 29.6, 29.3, 29.2, 29.0, 26.2, 22.7, 14.2. FT-IR (thin film) ν 2923, 2853, 1735, 1457, 1249, 1113, 669. To a suspension of the previous ester (2.00 g, 4.33 mmol) in water (40 mL) was added n-Bu₄NHSO₄ (284 mg, 0.866 mmol) and Na₂CO₃ (1.83 g, 17.30 mmol), and the reaction mixture was stirred for 20 min at room temperature affording a clear solution. Then, the mixture was cooled down to 0° C. and a solution of chloromethylchlorosulfate (569 μL, 5.62 mmol) in dry CH₂Cl₂ (40 mL) was added dropwise over 5 min. The resulting white suspension was vigorously stirred for 1 h at 0° C., and for 18 h at room temperature. The organic layer was decanted, and the aqueous phase was extracted with CH₂Cl₂ (2×100 mL). Combined organic layers were dried over Na₂SO₄, filtered and concentrated under reduced pressure. Compound MR27 was recovered as a yellow oil (2.19 g, 99%) and was used in the next step without further purification. TLC R_{f} 0.47 (CH₂Cl₂/MeOH 90:10). ¹H-NMR (400 MHz, CDCl₃) δ 5.68 (s, 2H), 4.24-4.21 (m, 2H), 3.68-3.59 (m, 16H), 3.41 (t, ³J_{HH}=6.8 Hz, 2H), 2.68 (s, 2H), 1.57-1.50 (m, 2H), 1.32-1.16 (m, 18H), 0.85 (t, ³J_{HH}=6.8 Hz, 3H). ¹³C-NMR (100 MHz, CDCl₃): δ 171.9, 170.6, 71.7, 70.8 (2C), 69.2, 68.9, 64.2, 32.1, 29.9, 29.8 (2C), 29.7, 29.6, 29.1, 28.9, 26.3, 22.8, 14.3. To a solution of DOPC (423 mg, 0.54 mmol) in freshly distilled CHCl₃ (10 mL) was added a solution of compound MR27 (2.19 g, 4.28 mmol) in dry CHCl₃ (5 mL). The resulting reaction mixture was heated at reflux for 20 h under a positive argon atmosphere, then cooled down and concentrated under reduced pressure at 20-25° C. The residue was purified over silica gel by flash chromatography (CH₂Cl₂/MeOH 88:12 to 80:20) to yield compound MR28 (147 mg, 21%) as a waxy solid (mixture of 2 diastereomers). TLC R_{f} 0.16 (CH₂Cl₂/MeOH 90:10). ¹H-NMR (400 MHz, CDCl₃) δ 5.74-5.61 (m, 2H), 5.37-5.26 (m, 5H), 4.56 (br, 2H), 4.44-4.12 (m, 8H), 3.85-3.75 (m, 2H), 3.65-3.53 (m, 18H), 3.40 (t, ³J_{HH}=6.9 Hz, 2H), 3.25 (s, 9H), 2.77-2.65 (m, 4H), 2.40-2.28 (m, 4H), 2.05-1.95 (m, 8H), 1.67-1.5 (m, 6H), 1.39-1.22 (m, 58H), 0.86 (t, ³J_{HH}=6.7 Hz, 9H). ¹³C-NMR (100 MHz, CDCl₃) δ 173.5, 173.1, 171.2, 130.3, 129.9, 83.2, 71.8, 70.8, 70.7, 70.2, 69.5, 69.1, 66.8, 66.6, 65.5, 64.4, 62.4, 61.7, 61.6, 54.8, 34.4, 34.2, 32.1, 29.9, 29.8, 29.7, 29.5, 29.4, 29.3, 28.9, 28.6, 27.4, 25.1, 22.9, 14.3. ³¹P-NMR (162 MHz, CDCl₃) δ −3.4, −3.6. ESI-MS (m/z): calc. for C₆₁H₁₁₅NO₁₂P(C₂H₄O)₄ 1260.92. found 1260.9. FT-IR (thin film) ν 2927, 2842, 1732, 1440, 1109, 969.

### Biological Evaluation of the Analogues

The ability to transfect siRNA into U87Luc cells and pCMVLuc pDNA into BHK-21 was assessed for each synthetized analogue as shown in parts A and B hereabove.

The cell toxicity of lipoplex was assessed by LDH release assay as described in part A and B hereabove.

1. Series 1: conjugates wherein R⁸ is

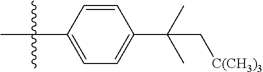 [SMILES: *c1ccc(C(C)(C)CC(C)(C)C)cc1]

1.a. SiRNA Transfection in U87Luc Cells

TABLE 5
Results for siRNA transfection

| | | | Percentage of residual luciferase expression | | | | | |
|---|---|---|---|---|---|---|---|---|
| | Features | | Luc-targeting siRNA | | | Control siRNA | | |
| N^{o} | PEG | R¹/R² | N/P: 25 | N/P: 50 | N/P: 100 | N/P: 25 | N/P: 50 | N/P: 100 |
| PP168 | 7 | C17, 1 unsat. | 90.88 | 44.00 | 12.31 | 95.25 | 77.69 | 34.17 |
| PP430 | 20 | C17, 1 unsat | 100.66 | 190.59 | 99.11 | 98.84 | 87.64 | 96.64 |
| PP431 | 9 | C15, 0 sat | 107.08 | 75.27 | 54.05 | 96.59 | 64.76 | 54.07 |

N/P: charge ratio (mole ammonium/mole phosphate),
PEG: number of monomer units,
unsat: unsaturated,
sat: saturated.

1.b. pDNA Transfection in BHK-21 Cells

TABLE 6
Results for pDNA transfection in BHK-21 cells

| | Features | | Luciferase expression (RLU/well) | | |
|---|---|---|---|---|---|
| N° | PEG | RVR² | N/P: 1 | N/P: 3 | N/P: 5 |
| PP168 | 7 | C17, 1 unsat. | 1.57 × 10⁵ | 7.32 × 10⁵ | 2.10 × 10⁶ |
| PP430 | 20 | C17, 1 unsat | 5.50 × 10³ | 3.32 × 10⁴ | 4.49 × 10⁴ |
| PP431 | 9 | C15, 0 sat | 4.90 × 10⁵ | 6.45 × 10⁶ | 6.75 × 10⁶ |

| | |
|---|---|
| Control* | 5.31 × 10³ |

N/P: charge ratio (mole ammonium/mole phosphate), PEG: number of monomer units, unsat: unsaturated, sat: saturated,
*control refers to basal luminescence of untreated cells.

1.c. Lipoplex toxicity

TABLE 7
results of LDH release

| | | | Percentage of LDH release | | | | | |
|---|---|---|---|---|---|---|---|---|
| | | | Lipoplex with siRNA | | | Lipoplex with pDNA | | |
| | Features | | (U87Luc) | | | (BHK-21) | | |
| N^{o} | PEG | R¹/R² | N/P: 25 | N/P: 50 | N/P: 100 | N/P: 1 | N/P: 3 | N/P: 5 |
| PP168 | 7 | C17, 1 unsat. | −0.68 | 15.02 | 31.34 | −0.61 | 0.52 | 2.86 |
| PP430 | 20 | C17, 1 unsat | −1.25 | −1.65 | −1.63 | −1.05 | −0.96 | −0.79 |
| PP431 | 9 | C15, 0 sat | −1.94 | −1.83 | 4.24 | −0.09 | 5.25 | 2.38 |

N/P: charge ratio (mole ammonium/mole phosphate),
PEG: number of monomer units,
unsat: unsaturated,
sat: saturated,
* control refers to basal luminescence of cells.

### 1.d Conclusion

PP430 is inefficient to transfect siRNA in U87Luc cells and displays a low pDNA transfection activity in BHK-21 cells. Such a result suggests that PEG spacer length may have some impact on transfection activity and that linker having more than 20 monomers should be preferably avoided when R⁸ is 2-(trimethylpentyl)phenyl moiety for nucleic acid transfection.

On the other hand, PP431 and PP168 display a similar efficiency for transfecting pDNA in cells. Such a result illustrates that some modification may be performed on R¹ and R² without impairing transfection activity.

### 2. Series 2: Conjugates Wherein R⁸ is a Linear Alkyl Chain, Influence of PEG Spacer and R8 Length

2.a. SiRNA Transfection in U87Luc Cells

TABLE 8
Results for siRNA transfection

| | | | | Percentage of residual luciferase expression | | | | | |
|---|---|---|---|---|---|---|---|---|---|
| | Features | | | Luc-targeting siRNA | | | Control siRNA | | |
| N^{o} | PEG | R⁸ | L? | N/P: 25 | N/P: 50 | N/P: 100 | N/P: 25 | N/P: 50 | N/P: 100 |
| PP514* | 0 | C₁₂H₂₅ | No | 103.75 | 101.21 | 99.76 | 100.51 | 98.50 | 94.58 |
| PP531 | 1 | C₁₂H₂₅ | No | 96.67 | 99.51 | 99.25 | 99.45 | 90.60 | 94.54 |
| PP529 | 3 | C₁₂H₂₅ | No | 93.74 | 48.65 | 21.74 | 102.03 | 77.00 | 67.21 |
| PP415 | 4 | C₁₂H₂₅ | No | 99.98 | 79.93 | 41.41 | 107.89 | 103.01 | 85.97 |
| PP426 | 7 | C₁₈H₃₇ | No | 106.48 | 82.13 | 62.14 | 102.88 | 94.43 | 87.31 |
| PP419 | 7 | C₆H₁₃ | No | 95.82 | 97.29 | 86.68 | 97.58 | 99.65 | 97.79 |
| PP418 | 7 | C₁₂H₂₅ | No | 109.46 | 89.32 | 55.05 | 100.08 | 94.25 | 86.23 |
| PP427 | 22 | C₁₂H₂₅ | No | 88.42 | 97.51 | 91.42 | 78.25 | 85.91 | 79.25 |
| PP522 | 7 | C₁₉H₃₇ | No | 90.10 | 62.38 | 21.41 | 95.46 | 79.37 | 49.87 |

N/P: charge ratio (mole ammonium/mole phosphate),
PEG: number of monomer units,
L?: presence of a hydrolyzable linker,
*P514 is a control compound.

2.b. DNA Transfection in BHK-21 Cells

TABLE 9
Results for pDNA transfection in BHK-21 cells

| | | | | Luciferase expression | | |
|---|---|---|---|---|---|---|
| | Features | | | (RLU/well) | | |
| N^{o} | PEG | R⁸ | L? | N/P: 1 | N/P: 3 | N/P: 5 |
| PP514* | 0 | C₁₂H₂₅ | No | 1.49 × 10⁷ | 1.26 × 10⁷ | 2.79 × 10⁷ |
| PP531 | 1 | C₁₂H₂₅ | No | 2.37 × 10⁷ | 3.37 × 10⁷ | 2.02 × 10⁷ |
| PP529 | 3 | C₁₂H₂₅ | No | 1.40 × 10⁸ | 1.76 × 10⁷ | 9.80 × 10⁷ |
| PP415 | 4 | C₁₂H₂₅ | No | 3.78 × 10⁷ | 1.10 × 10⁸ | 1.66 × 10⁸ |
| PP426 | 7 | C₁₈H₃₇ | No | 1.32 × 10⁶ | 2.49 × 10⁷ | 9.84 × 10⁶ |
| PP419 | 7 | C₆H₁₃ | No | 1.44 × 10⁶ | 9.67 × 10⁶ | 2.84 × 10⁷ |
| PP418 | 7 | C₁₂H₂₅ | No | 2.11 × 10⁶ | 8.02 × 10⁶ | 1.20 × 10⁷ |
| PP427 | 22 | C₁₂H₂₅ | No | 5.49 × 10⁴ | 5.37 × 10⁴ | 9.26 × 10⁴ |
| PP522 | 7 | C₁₉H₃₇ | No | 9.34 × 10⁶ | 2.85 × 10⁷ | 6.68 × 10⁶ |

| | |
|---|---|
| Control** | 3.06 × 10⁴ |

N/P: charge ratio (mole ammonium/mole phosphate),
PEG: number of monomer units,
L?: presence of a hydrolyzable linker,
*P514 is a control compound,
**control refers to basal luminescence of untreated cells.

2.c. Lipoplex Toxicity

TABLE 10
results of LDH release

| | | | | Percentage of LDH release | | | | | |
|---|---|---|---|---|---|---|---|---|---|
| | | | | Lipoplex with siRNA | | | Lipoplex with pDNA | | |
| | Features | | | (U87Luc) | | | (BHK-21) | | |
| N^{o} | PEG | R⁸ | L? | N/P: 25 | N/P: 50 | N/P: 100 | N/P: 1 | N/P: 3 | N/P: 5 |
| PP514* | 0 | C₁₂H₂₅ | No | −2.36 | −3.13 | −3.29 | 0.15 | 0.76 | 0.46 |
| PP531 | 1 | C₁₂H₂₅ | No | −1.65 | −4.87 | −2.33 | −0.22 | −1.08 | −0.05 |
| PP529 | 3 | C₁₂H₂₅ | No | 2.26 | 1.10 | 16.50 | 2.64 | 0.31 | 15.92 |
| PP415 | 4 | C₁₂H₂₅ | No | −1.47 | 10.56 | 25.09 | 1.02 | 4.08 | 14.26 |
| PP426 | 7 | C₁₈H₃₇ | No | −0.91 | −1.14 | 6.60 | −0.30 | 4.45 | 17.69 |
| PP419 | 7 | C₆H₁₃ | No | −1.84 | −1.18 | −1.07 | 0.15 | 0.6 | 7.77 |
| PP418 | 7 | C₁₂H₂₅ | No | −1.18 | −1.54 | −0.03 | −0.71 | 1.74 | 15.99 |
| PP427 | 22 | C₁₂H₂₅ | No | −0.06 | −0.19 | −0.49 | 0.39 | 0.34 | 0.08 |
| PP522 | 7 | C₁₉H₃₇ | No | −1.42 | 2.64 | 20.12 | 0.46 | 5.43 | 17.42 |

N/P: charge ratio (mole ammonium/mole phosphate),
PEG: number of monomer units,
L?: presence of a hydrolyzable linker,
*P514 is a control compound.

2.d. Conclusion

PP514, which contains no PEG moiety and no hydrolyzable connector, is able to transfect DNA into cells (BHK-21) but is totally inefficient to transfect siRNA (U87Luc). The introduction of a PEG moiety (comprising at least 2 monomer units) and/or that of a hydrolyzable connector (see part B hereabove) is able to restore some transfection activity in respect to siRNA molecules. PP415 (4 monomers), PP529 (3 monomers) and PP418 (7 monomers) are efficient to transfect both siRNA and pDNA. However, PP427 (22 monomers) display poor transfection activity, which illustrates that spacer containing more than 22 monomers should be avoided when R⁸ is nC₁₂H₂₅. One may deduce that oligoethylene oxide comprising at least 2 monomers and less than 22 monomers should be preferred in order to get compounds having both significant DNA and RNA transfection activity.

The length of R⁸ group may have also some impact on transfection activity: The highest DNA activity is obtained for PP415 (nC₁₂H₂₅) Indeed, PP419 (C₆H₁₃) and PP426 (C₁₈H₃₇) display significant but lower DNA transfection activity in BHK-21 cells. The impact is more visible for siRNA transfection: PP419 displays very low transfection activity whereas the apparent efficiency of PP426 may be explained by a higher toxicity as illustrated by LDH release assay.

3. Series 2: analogues of PP415 comprising an hydrolyzable linker
3.a. pDNA Transfection in BHK-21 Cells

TABLE 11
Results for pDNA transfection in BHK-21 cells

| | | | | Luciferase expression | | |
|---|---|---|---|---|---|---|
| | Features | | | (RLU/well) | | |
| N^{o} | PEG | R⁸ | L | N/P: 1 | N/P: 3 | N/P: 5 |
| MR22 | 4 | C₁₂H₂₅ | —CH₂OC(O)O— | 3.00 × 10⁷ | 8.24 × 10⁵ | 5.19 × 10⁵ |
| MR25 | 4 | C₁₂H₂₅ | —CH(Me)—O—C(O)O— | 3.30 × 10⁶ | 5.32 × 10⁵ | 3.56 × 10⁴ |
| MR26 | 4 | C₁₂H₂₅ | —CH(iPr)—O—C(O)O— | 6.99 × 10⁶ | 1.00 × 10⁶ | 1.19 × 10⁵ |
| MR28 | 4 | C₁₂H₂₅ | CH₂O—C(O)—CH₂—CH₂—C(O)O— | 7.33 × 10⁶ | 5.69 × 10⁵ | 1.20 × 10⁵ |
| PP415 | 4 | C₁₂H₂₅ | none | 5.78 × 10⁷ | 2.39 × 10⁷ | 9.89 × 10⁶ |

| | |
|---|---|
| Control* | 4.98 × 10³ |

N/P: charge ratio (mole ammonium/mole phosphate),
PEG: number of monomer units,
L?: presence of a hydrolyzable linker,
*Measured for untreated cells.

3.b. Lipoplex Toxicity

TABLE 12
Results of LDH release

| | | | | Percentage of | | |
|---|---|---|---|---|---|---|
| | Features | | | LDH release | | |
| N^{o} | PEG | R⁸ | L | N/P: 1 | N/P: 3 | N/P: 5 |
| MR22 | 4 | C₁₂H₂₅ | —CH₂OC(O)O— | 6.54 | 5.91 | 6.65 |
| MR25 | 4 | C₁₂H₂₅ | —CH(Me)—O—C(O)O— | 0.87 | 6.05 | 6.73 |
| MR26 | 4 | C₁₂H₂₅ | —CH(iPr)—O—C(O)O— | 2.53 | 7.44 | 7.33 |
| MR28 | 4 | C₁₂H₂₅ | CH₂O—C(O)—CH₂—CH₂—C(O)O— | 1.28 | 5.18 | 7.00 |
| PP415 | 4 | C₁₂H₂₅ | none | 7.85 | 9.38 | 14.88 |

N/P: charge ratio (mole ammonium/mole phosphate),
PEG: number of monomer units,
L?: presence of a hydrolyzable linker.

Notably, all the above analogues of PP415 display significant DNA transfection activity and lower toxicity since the percentage of LDH release is less than 10%.

### REFERENCES

[1] Feigner P L, Gadek T R, Holm M, Roman R, Chan H W, Wenz M, et al. Proc Natl Acad Sci USA 1987; 84:7413-7.
[2] Wu G Y, Wu C H. J Biol Chem 1987; 262:4429-32.
[3] Fire A, Xu S Q, Montgomery M K, Kostas S A, Driver S E, Mello C C. Nature 1998; 391:806-11.
[4] Elbashir S M, Harborth J, Lendeckel W, Yalcin A, Weber K, Tuschl T. Nature 2001; 411:494-8.
[5] Li W, Szoka F. Pharm Res 2007; 24:438-49.
[6] Hillaireau H, Couvreur P. Cell Mol Life Sci 2009; 66:2873-96.
[7] Reischl D, Zimmer A. Nanomed Nanotech Biol Med 2009; 5:8-20.
[8] David S, Pitard B, Benoit J P, Passirani C. Pharmacol Res 2010; 62:100-14.
[9] Sawant R R, Torchilin V P. Soft Matter 2010; 6:4026-44.
[10] Tros de Iiarduya C, Sun Y, Düzgünes N. Eur J Pharm Sci 2010; 40:159-70.
[11] Dominska M, Dykxhoorn D M. Breaking down the barriers: siRNA delivery and endosome escape. J Cell Sci 2010; 123:1183-9.
[12] Nie L H, Zhao H C, Wang X, Yi L, Lu Y, Jin L P, et al. Anal Bioanal Chem 2002; 374:1187-90.
[13] Lu J J, Langer R, Chen J. Mol Pharm 2009; 6:763-71.
[14] Zou S, Scarfo K, Nantz M H, Hecker Int J Pharm 2010; 389:232-43.
[15] Tarahovsky Y S. Biochem—Moscow 2010; 75:811-24.
[16] Khalil I A, Kogure K, Akita H, Harashima H. Pharmacol Rev 2006; 58:32-45.
[17] Mastrobattista E, van der Aa M, Hennink W E, Nature Rev Drug Discov 2006; 5:115-21.
[18] Gruenberg J. TNat Rev Mol Cell Biol 2001; 2:721-30.
[19] Gruenberg J, Griffiths G, Howell K E. J Cell Biol 1989; 108:1301-16.
[20] Mukherjee S, Soe T T, Maxfield F R. J Cell Biol 1999; 144:1271-84.
[21] Cevc G, Richardsen H. Adv Drug Deliv Rev 1999; 38:207-32.
[22] Wagner E. Adv Drug Deliv Rev 1999; 38:279-89.
[23] Lezoualch F, Seugnet I, Monnier A L, Ghysdael J, Behr J P, Demeneix B A. J Biol Chem 1995; 270:12100-8.
[24] Lechardeur D, Sohn K J, Haardt M, Joshi P B, Monck M, Graham R W, et al. Gene Ther 1999; 6:482-97.
[25] Wong F M P, Reimer D L, Bally M B. Biochemistry 1996; 35:5756-63.
[26] Behr J P. Tetrahedron Lett 1986; 27:5861-4.
[27] Mel'nikov S M, Sergeyev V G, Yoshikawa K. J Am Chem Soc 1995; 117:2401-8.
[28] Pinnaduwage P, Schmitt L, Huang L. Biochim Biophys Acta 1989; 985:33-7.
[29] Rose J K, Buonocore L, Whitt M A. 1991; 10:526-32.
[30] Clamme J P, Bernacchi S, Vuilleumier C, Duportail G, Mely Y. Biochim Biophys Acta 2000; 1467:347-61.
[31] Hattori Y, Ding W X, Maitani Y. J Control Release 2007; 120:122-30.
[32] Choi S H, Jin S E, Lee M K, Lim S J, Park J S, Kim B G, et al. Eur J Pharm Biopharm 2008; 68:545-54.
[33] Ding W X, Izumisawa T, Hattori Y, Qi X R, Kitamoto D, Maitani Y. Biol Pharm Bull 2009; 32:311-5.
[34] Pierrat P, Creusat G, Laverny G, Pons F, Zuber G, Lebeau L. Chem-Eur J 2012; 18:3835-3839.
[35] Gorman C M, Aikawa M, Fox B, Fox E, Lapuz C, Michaud B, et al. Gene Ther 1997; 4:983-92.
[36] MacDonald R C, Ashley G W, Shida M M, Rakhmanova V A, Tarahovsky Y S, Pantazatos D P, et al. Biophys J 1999; 77:2612-29.
[37] Kennedy M T, Pozharski E V, Rakhmanova V A, MacDonald R C. Biophys J 2000; 78:1620-33.
[38] Rakhmanova V A, McIntosh T J, MacDonald R C. Cell Mol Biol Lett 2000; 5:51-65.
[39] Rosenzweig H S, Rakhmanova V A, McIntosh T J, MacDonald R C. Bioconjugate Chem 2000; 11:306-13.
[40] Koynova R, MacDonald R C. Cationic Biochim Biophys Acta 2003; 1613:39-48.
[41] Koynova R, Wang L, MacDonald R C. Proc Natl Acad Sci USA 2006; 103:14373-8.
[42] MacDonald R C, Gorbonos A, Mornsen M M, Brockman H L. Langmuir 2006; 22:2770-9.
[43] Wang L, Koynova R, Parikh H, MacDonald R C. Biophys J 2006; 91:3692-706.
[44] Tenchov B G, Wang L, Koynova R, MacDonald R C. Miochim Biophys Acta 2008; 1778:2405-12.
[45] Koynova R, Tenchov B, Wang L, MacDonald R C. Mol Pharm 2009; 6:951-8.
[46] Lebeau L, Olland S, Oudet P, Mioskowski C. Chem Phys Lipids 1992; 62:93-103.
[47] Gentine P, Bubel A, Crucifix C, Bourel-Bonnet L, Frisch B. J Liposome Res 2011; 22:18-30.
[48] Creusat G, Rinaldi A S, Weiss E, Elbaghdadi R, Remy J S, Mulherkar R, et al. Bioconjugate Chem 2010; 21:994-1002.
[49] Mukherjee S, Ghosh R N, Maxfield F R. Physiol Rev 1997; 77:759-803.
[50] Bareford L M, Swaan P W. Adv Drug Deliv Rev 2007; 59:748-58.
[51] Kreevoy M M, Taft R W. T J Am Chem Soc 1955; 77:5590-5.
[52] Srinivasachar K, Neville D M. Biochemistry 1989; 28:2501-9.
[53] Farquhar D, Srivastva D N, Kuttesch N J, Saunders P P. J Pharm Sci 1983; 72:324-5.
[54] Srivastva D N, Farquhar D. Bioorg Chem 1984; 12:118-29.
[55] Schultz C, Vajanaphanich M, Harootunian A T, Sammak P J, Barrett K E, Tsien R Y. J Biol Chem 1993; 268:6316-22.
[56] Schultz C, Vajanaphanich M, Genieser H-G, Jastorff B, Barrett K E, Tsien R Y. Mol Pharmacol 1994; 46:702-8.
[57] Friis G J, Bundgaard H. Prodrugs of phosphates and phosphonates: Eur J Pharm Sci 1996; 4:49-60.
[58] Kruppa J, Keely S, Schwede F, Schultz C, Barrett K E, Jastorff B. Bioorg Med Chem Lett 1997; 7:945-8.
[59] Lindahl A, Malmberg M, Rehnberg N. J Carbohydr Chem 1996; 15:549-54.
[60] Gunic E, Girardet J-L, Ramasamy K, Stoisavljevic-Petkov V, Chow S, Yeh L-T, et al. Bioorg Med Chem Lett 2007; 17:2452-5.
[61] Prata C A H, Zhao Y, Barthelemy P, Li Y, Luo D, McIntosh T J, et al. J Am Chem Soc 2004; 126:12196-7.
[62] Zhang X X, Allen P G, Grinstaff M. Mol Pharm 2011; 8:758-66.
[63] Zhang X X, Prata C A H, Berlin J A, McIntosh T J, Barthelemy P, Grinstaff M W. Bioconjugate Chem 2011; 22:690-9.
[64] Boden N, Bushby R J, Clarkson S, Evans S D, Knowles P F, Marsh A. Tetrahedron 1997; 53:10939-52.
[65] Loiseau F A, Hii K K, Hill A M. J Org Chem 2004; 69:639-47.
[66] Zervas L, Dilaris I. J Am Chem Soc 1955; 77:5354-7.
[67] Savignac P, Lavielle G. Bull Soc Chim Fr 1974:1506-8.
[68] Klein E, Mons S, Valleix A, Mioskowski C, Lebeau L. J Org Chem 2002; 67:146-53.
[69] Klein E, Nghiem H O, Valleix A, Mioskowski C, Lebeau L. Chem Eur J 2002; 8:4649-55.
[70] Pop E, Wu W M, Bodor N. J Med Chem 1989; 32:1789-95.
[71] Baudy R B, Butera J A, Abou-Gharbia M A, Chen H, Harrison B, Jain U, et al. J Med Chem 2009; 52:771-8.
[72] van Dijk-Wolthuis W N E, vanSteenbergen M J, Underberg W J M, Hennink W E. J Pharm Sci 1997; 86:413-7.
[73] Ostergaard J, Larsen C. BMolecules 2007; 12:2396-412.
[74] Foillard S, Zuber G, Doris E. Nanoscale 2011; 3:1461-4.
[75] Caplen N J, Parrish S, Imani F, Fire A, Morgan R A. Proc Natl Acad Sci USA 2001; 98:9742-7.
[76] Mosmann T. J Immunol Methods 1983; 65:55-63.
[77] Heeg K, Reimann J, Kabelitz D, Hardt C, Wagner H. J Immunol Methods 1985; 77:237-46.
[78] MacDonald R C, Rakhmanova V A, Choi K L, Rosenzweig H S, Lahiri M K. J Pharm Sci 1999; 88:896-904.

1-15. (canceled)

16. A compound of formula (I): 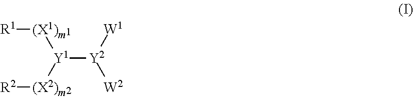
or a salt thereof, wherein:
R¹ and R² are independently selected from the groups consisting of linear, unsaturated or saturated, C₈ to C₃₀ alkyl groups optionally interrupted by one or several heteroatoms and eventually substituted by one or several groups selected from C₁-C₃ alkyl groups, halogens, —OH, —OMe, and —CF₃,
X¹ and X² are independently selected from the group consisting of —O—, —OC(O)—, —C(O)O—, —OC(O)O—, —S—, —SS—, —SC(O)—, —OC(S)—, —NR³—, —NR³C(O)—, —C(O)NR³—, —NR³C(S)—, —C(S)NR³—, —OC(O)S—, —OC(S)O—, —SC(O)O—, —OC(S)S—, —SC(O)S—, —SC(S)O—, —SC(S)S—, —OC(O)NR³—, —OC(S)NR³—, —NR³C(S)O—, —NR³C(O)S—, —NR³C(O)NR⁴—, —NR³C(S)NR⁴—, —SC(O)S—, —SC(S)O—, —S(O)—, —S(O)₂—, —O(CR³R⁴)O—, —C(O)O(CR³R⁴)O—, —OC(O)O(CR³R⁴)O—, —P(O)(R³)—, —P(O)(OR³)—, —P(O)(R³)O—, —OP(O)(OR³)—, —OP(O)(R³)O—, —NR³P(O)(R⁴)—, —NR³P(O)(OR⁴)—, —NR³P(O)(R⁴)O—, —OP(O)(OR³)— and —OP(O)(R³)O— wherein R³ and R⁴ are independently H or CH₃,
m¹ and m² are integers independently selected from 0 and 1,
Y¹ and Y² are trivalent connectors selected from the group consisting of —N<, —CON<, 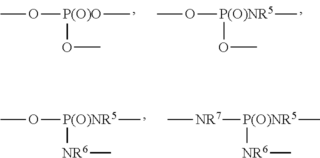
and linear or branched alkyl C₁-C₁₀ groups, wherein R⁵, R⁶ and R⁷ are independently selected from H and CH₃,
W² is a straight or branched radical comprising from 2 to 20 carbon atoms and at least one functional group selected from ester, carboxylate, —OH, ether, primary, secondary, tertiary or quaternary amine and combinations thereof;
W¹ is a radical having the following formula (II):
-(L)ₛ-(ZO)ₙ—R⁸
wherein:
s is 0 or 1,
n is an integer from 0 to 30 or from 0 to 22 with the proviso that n is not 0 when s is 0,
L is —C(R²⁰)(R²¹)—O—C(O)—, —C(R²²)(R²³)—O—C(O)—O— or C(R²⁴)(R²⁵)—O—C(O)—(CH₂)ₚ—C(O)O— wherein:
R²⁰ to R²⁵ are selected from the group consisting of H and C¹-C³ alkyl groups, which may be linear, cyclic or branched, and
p is an integer between 1 to 10,
Z is —CH₂CH₂—, —CH₂CH₂CH₂— or —CH₂CH₂CH₂CH₂— and
R⁸ is selected from the group consisting of:
unsaturated or saturated linear C₁-C₂₄ alkyl groups, or C₅-C₂₄ groups, optionally substituted with one or several groups selected from —F, —Cl, —Br, —I, —OH, —OMe, C₁-C₄ alkyl groups and —CF₃ and optionally interrupted by a heteroatom; and
an aryl group substituted by one or several C₁-C₁₂ linear or branched alkyl groups, or

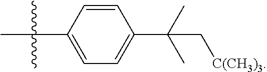 [SMILES: *c1ccc(C(C)(C)CC(C)(C)C)cc1]

17. The compound according to claim 16, wherein the said compound is of formula (IV) 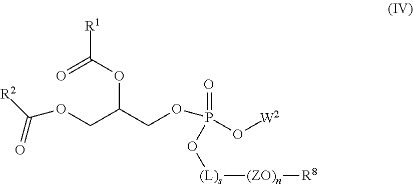
wherein R¹, R², L, W², s, Z and R⁸ are as defined in claim 16.

18. The compound according to claim 17, wherein:
s=1 and
L is selected from the group consisting of —CH₂—O—C(O)—(CH₂)₂—C(O)O—, —CH₂—O—C(O)—, and —CH(R²²)—O—C(O)—O— wherein R²² is —H, —CH₃ or —CH(CH₃)₂.

19. The compound according to claim 17, wherein R⁸ is selected from the group consisting of:
—(CH₂)ₓCH₃ with x an integer from 0 to 23 or from 0 to 16, and
—(CH₂)_{y}—CH═CH—(CH₂)_{z}—CH₃ with z and y are integers such that 2≦y+z≦21.

20. The compound according to claim 19, wherein the said compound is characterized by one of the following combinations of features:
i) s=0, Z=—CH₂—CH₂— and n is an integer from 1 to 8; and
ii) s=1, Z=—CH₂—CH₂—, n is an integer from 0 to 8 and L is —CH₂—O—C(O)—, —CH₂—O—C(O)—O—, —CH(CH₃)—O—C(O)—O—, —CH(iPr)—O—C(O)—O— and CH₂—O—C(O)—CH₂—CH₂—C(O)—O—.

21. The compound of claim 17, wherein R⁸ is

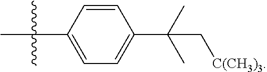 [SMILES: *c1ccc(C(C)(C)CC(C)(C)C)cc1]

22. The compound according to claim 21, wherein:
Z is —CH₂—CH₂—,
n is an integer from 5 to 10, and
s=0 or s=1 with the proviso that L is —CH₂—O—C(O)—(CH₂)₂—C(O)—O—, —CH(CH₃)—O—C(O)O— or CH₂—O—C(O)O—.

23. The compound according to claim 16, wherein W² is selected from the group consisting of:
—CH₂—CH(COOH)NH₂,
—CH₂—CH(OH)—CH₂—OH,
a straight or branched oligoethylenimine comprising from 2 to 6 monomers,
—(Z¹NR¹⁴)_{q}—R¹⁵ and
—Z¹NR¹⁶R¹⁷R¹⁸⁺Q⁻;
wherein Z¹ is the same or different and selected from the group consisting of —(CH₂)₂—, —(CH₂)₃— or —(CH₂)₄—, q is an integer from 1 to 4, R¹⁴ to R¹⁸ is H or CH₃ and Q⁻ is a pharmaceutically acceptable anion.

24. The compound according to claim 16, wherein R¹ and R² are independently selected from unsubstituted and straight C¹²-C²⁴ alkyl groups comprising 0, 1, 2, 3 or 4 unsaturations.

25. The compound according to claim 16, wherein R¹ and R² are CH₃—(CH₂)₇—CH═CH—(CH₂)₇— and W² is —CH₂—CH₂—N(CH₃)₃⁺Q-.

26. A supramolecular complex comprising one or several compounds as defined in claim 16, and a pharmaceutically active compound.

27. The supramolecular complex according to claim 26, wherein the active compound is a nucleic acid molecule.

28. The supramolecular complex according to claim 26, wherein the nucleic acid molecule is a siRNA or a DNA molecule.

29. A pharmaceutical composition comprising a pharmaceutically active compound, a compound as defined in claim 16, and optionally a pharmaceutically acceptable excipient.

30. The pharmaceutical composition according to claim 29, wherein the active compound is a nucleic acid molecule.

31. The pharmaceutical composition according to claim 30, wherein the nucleic acid molecule is a siRNA or a DNA molecule.

32. A method for delivering a molecule of interest to a cell, said method comprising contacting a supramolecular complex as defined in claim 26 or a pharmaceutical composition thereof with said cell.

33. A method for administering a pharmaceutically active compound to an animal, said method comprising administering a supramolecular complex as defined in claim 26 or a pharmaceutical composition thereof to said animal.


SEQUENCE LISTING (2 sequences)

SEQ ID NO: 1 - RNA, 19 nt, Artificial Sequence
  sense strand of siRNA (siLuc)
cuuacgcugaguacuucga

SEQ ID NO: 2 - RNA, 19 nt, Artificial Sequence
  sense strand of untargeted siRNA (sic)
cguacgcggaauacuucga